(19) United States
(12) Patent Application Publication
(10) Pub. No.: US 2021/0030901 A1
(43) Pub. Date: Feb. 4, 2021

(54) INORGANIC NANOCAGES, AND METHODS OF MAKING AND USING SAME

(71) Applicant: Cornell University, Ithaca, NY (US)

(72) Inventors: Ulrich B. Wiesner, Ithaca, NY (US); Melik Z. Turker, Princeton, NJ (US); Kai Ma, Belle Mead, NJ (US); Tangi Aubert, Ithaca, NY (US)

(21) Appl. No.: 17/064,352

(22) Filed: Oct. 6, 2020

Related U.S. Application Data
(63) Continuation-in-part of application No. PCT/US2019/026411, filed on Apr. 8, 2019.
(60) Provisional application No. 63/071,271, filed on Aug. 27, 2020.
(60) Provisional application No. 62/653,803, filed on Apr. 6, 2018.

(51) Int. Cl. A61K 51/12; C01B 33/18; C09C 1/30

(52) U.S. Cl. CPC A61K 51/1251; C01B 33/18; C09C 1/3072; B82Y 40/00; C01P 2004/64; C01P 2006/12; C01P 2004/04; C01P 2004/34

(57) ABSTRACT

Provided are inorganic nanocages. The inorganic nanocages may be non-metal nanocages, transition metal oxide nanocages, or transition metal nanocages. Non-metal nanocages may include metal oxides. The inorganic nanocages can be made using micelles formed using pore expander molecules. The inorganic nanocages may be used as catalysts, drug delivery agents, diagnostic agents, therapeutic agents, and theranostic agents.

### CROSS-REFERENCE TO RELATED APPLICATIONS

This application claims priority to U.S. Provisional Application No. 63/071,271, filed on Aug. 27, 2020, and is a continuation-in-part of International Application No. PCT/US2019/026411, filed on Apr. 8, 2019, which claims priority to U.S. Provisional Application No. 62/653,803, filed on Apr. 6, 2018, the disclosure of which are hereby incorporated by reference.

### STATEMENT REGARDING FEDERALLY SPONSORED RESEARCH

This invention was made with government support under Grant Number CA199081 awarded by the National Institutes of Health. The government has certain rights in the invention.

### FIELD OF THE DISCLOSURE

The disclosure generally relates to inorganic nanocages. The disclosure also relates to methods of making and methods of using inorganic nanocages.

### BACKGROUND OF THE DISCLOSURE

Nanoscale objects with highly symmetrical cage-like polyhedral shapes, often with icosahedral symmetry, have recently been assembled using DNA, RNA and proteins for biomedical applications. These achievements relied on advances in the development of programmable self-assembling biomaterials, as well as rapidly developing single-particle three-dimensional (3D) reconstruction techniques of cryo electron microscopy (cryo-EM) images that provide high-resolution structural characterization of biological complexes. In contrast, such single-particle 3D reconstruction approaches have not been successfully applied to help identify unknown synthetic inorganic nanomaterials with highly symmetrical cage-like shapes.

Topology is a topic across a wide range of scientific disciplines. While effects of size, shape, or composition of nanomaterials on biological response have been widely studied, much less is known about how topology modulates biological properties.

There is an ongoing and unmet need for inorganic cage-like materials.

### SUMMARY OF THE DISCLOSURE

The present disclosure provides inorganic nanocages. The present disclosure also provides methods of making and using the inorganic nanocages.

There are a number of important ramifications that derive from our inorganic nanocage discovery. The chemical and practical value of the polyhedral structures are considered to be extremely high. Considering the high versatility of, for example, silica, aluminosilicate, transition metal, and transition metal oxide surface chemistry, and the ability to distinguish cage inside and outside via micelle directed synthesis, one can readily conceive cage derivatives of many kinds, which may exhibit unusual properties and be useful in applications ranging from catalysis to drug delivery. For example, based on recent successes in clinical translation of ultrasmall fluorescent silica nanoparticles with similar particle size and surface properties, a whole range of novel diagnostic and therapeutic probes with drugs, which may be toxic drugs, hidden in the inside of the cages can be envisaged.

In an aspect, the present disclosure provides methods of making inorganic nanocages. A method may be based on self-assembly of inorganic nanocages.

Inorganic nanocages may be produced through self-assembly. Briefly, under the optimized synthesis conditions, inorganics self-assemble into the highly-symmetric cage structures on the surface of self-assembled surfactant micelles.

The functional group(s) carried by the inorganic nanocages can include diagnostic and/or therapeutic agents (e.g., radioisotopes, drugs, nucleic acids, and the like). An inorganic nanocage may comprise a combination of different functional groups.

Non-limiting examples of therapeutic agents, which may be drugs, include, but are not limited to, chemotherapeutic agents, antibiotics, antifungal agents, antiparasitic agents, antiviral agents, and combinations thereof, and groups derived therefrom. Examples of suitable drugs/agents are known in the art.

In an aspect, the present disclosure provides inorganic nanocages. The inorganic nanocages may be produced by a method of the present disclosure.

The inorganic nanocages are discrete nanoscale structures. The inorganic nanocages may have cage-like polyhedral shapes, which may have icosahedral symmetry. The inorganic nanocages comprise a plurality of polygons that form the inorganic nanocage. The polygons may all have the same shape or two or more of the polygons have different shapes. For example, the inorganic nanocages comprise the following surface polygons (where the exponent describes how often a polygon appears on the surface of the cage): 3³4³, 4⁴5⁴, 4³5⁶6³, 3³4³5⁹, 5¹² (dodecahedral), 5¹²6² 4⁶6⁸, 5¹²6³, 5¹²6⁴, 4³5⁹6²7³, 5¹²6⁸, 5¹²6²⁰ (buckyball) or the like.

The inorganic nanocages may comprise non-metal atoms in an oxidized state, metal atoms in an oxidized state (e.g., in the case of aluminosilicate nanocages), transition metal atoms in a neutral state or oxidized state, and combinations thereof. The inorganic nanocages may also comprise oxygen atoms. The inorganic nanocages may be non-metal oxide nanocages, transition metal nanocages, and transition metal oxide nanocages.

The inorganic nanocages may have several structural features. These features may include an interior, a plurality of apertures (which may be referred to as “windows” or “open windows”), arms (which may be referred to as “struts” or “edges”), and vertices. Examples of structural features are shown in FIG. 3.

In an aspect, the present disclosure provides compositions comprising inorganic nanocages of the present disclosure. The compositions can comprise one or more type(s) (e.g., having different average size and/or one or more different compositional feature(s)).

In an aspect, the present disclosure provides uses of inorganic nanocages. In various examples, inorganic nanocages or a composition comprising inorganic nanocages are used in delivery and/or imaging methods.

### BRIEF DESCRIPTION OF THE FIGURES

For a fuller understanding of the nature and objects of the disclosure, reference should be made to the following detailed description taken in conjunction with the accompanying figures.

FIG. 1 shows a representation of dodecahedron. Among the platonic solids, the dodecahedron best fills out its circumscribed sphere, i.e. a sphere that passes through all its vertices (left). The inscribed sphere passing through all facets (right) is shown for comparison.

FIG. 2 shows TEM and cryo-EM characterizations of silicages. a, TEM images at low magnification of PEG-coated silicages on carbon substrate. The inset in (a) shows a zoomed-in image. b, Averaged TEM image using eleven images acquired of the same sample area of PEG-coated silicages with insets showing representative individual structures at higher magnification. The sample was plasma etched for five seconds prior to TEM characterization to reduce background noise. c, Comparison between silicages observed in TEM and cryo-EM (revealing the lower background noise of cryo-EM images) with projections of simulated dodecahedral cages and models. d, Cryo-EM images of silicages without PEG coating. Scale bars in the insets in (b, c and d) are 5 nm.

FIG. 3 shows single particle reconstruction of dodecahedral silicage. a and b, Dodecahedral silicage reconstruction result (a) and its three most unique projections along the two-, three- and five-fold symmetry axes (b). The average dimensions of silicages, including outer and inner cage diameters, edge length, vertex diameter and window diameter, were estimated based on the reconstructed dodecahedral silicage (b). c, Representative comparison of nine unique projections from the reconstruction and cryo-EM cluster averages with projections of a 3D dodecahedral cage model (top row in c). Corresponding single cryo-EM images are displayed at the bottom in (c) highlighting the difference between raw data and reconstruction. Scale bar in (c) is 10 nm. Visualizations in panels (a) and (b) are by UCSF Chimera.

FIG. 4 shows cage-like structures with different inorganic compositions. a, b, and c, Similar cage-like nanoparticles were obtained when silica was replaced by other materials, including gold (a), silver (b), and vanadium oxide (c). The insets display zoomed-in images of individual particles (top two rows) and averaged images (bottom row). The scale bars in all insets are 2 nm.

FIG. 5 shows PEGylated silicages after cleaning and nitrogen sorption measurements on calcined cages. a, Representative dry-state TEM images at different magnifications of PEGylated silicages after the removal of surfactant and TMB. The black arrow indicates a PEGylated particle that exhibits cage-like structure, demonstrating the possibility of structure preservation after the removal of CTAB and surfactant. b, Nitrogen absorption and desorption isotherms of calcined silicages. After the removal of surfactant and TMB, particles were calcined at 550° C. for 6 hours in air prior to nitrogen sorption measurements. A particle synthesis yield of 67% was estimated from the weight of the calcined powder. The surface area of calcined silicages as assessed by the Brunauer-Emmett-Teller (BET) method was 570 m²/g, consistent with theoretical estimations.

FIG. 6 shows cluster averages of 2D images of silicages. a, 19,000 single particle cryo-EM images were sorted into 100 clusters. b, Some of the projections (examples highlighted in a) exhibited features similar to projections of dodecahedral cage structure obtained by simulation. Also shown are projection models. The scale bars are 10 nm.

FIG. 7 shows reconstruction of silicage using RELION 2.1 system. Dodecahedral silicage reconstruction result (a) and its three most unique projections along the two-, three- and five-fold symmetry axes (b). The reconstruction was obtained from a single-class calculation run by RELION 2.1 using the same set of single particle images as was used in the class of the dodecahedral cage shown in FIG. 3a. Visualization is by UCSF Chimera.

FIG. 8 shows a typical CTF and determination of reconstruction resolution. CTFFIND4.1.8 was used to estimate defocus for individual micrographs or set of micrographs with results consistent with the nominal defocus values of 1 to 2 microns. (a) Contrast transfer function (CTF) for defocus 1.98 microns. Since the first zero-crossing of CTF occurs at 0.44 nm⁻¹, the CTF has little effect on reconstructions unless the resolution is greater than 1/0.44=2.27 nm. (b) Fourier Shell Correlation (FSC) computed by standard package for two Hetero reconstructions that are independent starting at the level of separate sets of images each containing 2000 images (i.e., “gold standard” FSC). The resolution implied by the FSC curve (at 0.5 threshold) is 1/0.77=1.30 nm. (c) Energy function for the same pair of reconstructions as in (b). Energy is the spherical average of the squared magnitude of the reciprocal-space electron scattering intensity, where the denominator of FSC is the square root of a product of two Energy functions, one for each reconstruction. The observations that Energy has dropped by more than 10⁻³ times its peak value and the character of the curve has become oscillatory and more slowly decreasing, both by 0.44 nm⁻¹, indicates that the resolution implied by the FSC curve (at 0.5 threshold) is exaggerated and that a more conservative resolution is 1/0.44=2.27 nm. (d) FSC computed by a standard package for two RELION 2.1 reconstructions computed from the same images as the reconstructions in (b), from which the resolution (at 0.5 threshold) is estimated to be around 1/0.50=2.00 nm.

FIG. 9 shows probability analysis of silicage projections. a, Orientation dependence of silicage projections. The orientations, at which the nine different silicage projections (right panel) can be seen, are calculated, and manually mapped on a surface of a dodecahedron (left panel). The orientations, corresponding to different projections, are assigned to different colors. b, Probability analysis for different silicage projections. The probability of imaging a particular projection in EM is estimated by dividing that subset of the surface area of a sphere which contains the orientations that correspond to the specific projection, by the total surface area of the sphere (a). c, Experimental probability of different silicage projections. The probability of each projection is calculated by dividing the number of the single particle images assigned to the specific silicage projection via 3D reconstruction by the overall number of silicage single particle images.

FIG. 10 shows size analysis of silica clusters at an early stage of cage formation. Particle size distribution for primary silica clusters at an early stage of cage formation, obtained by manually analyzing 450 silica clusters using a set of TEM images. A representative TEM image is included in the inset. In order to quench the very early stages of cage formation, PEG-silane was added into the synthesis mixture about three minutes after the addition of TMOS thereby PEGylating early silica structures. TEM sample preparation and characterization were as described before. Primary silica clusters with diameters around 2 nm were identified, consistent with the proposed cage formation mechanism.

FIG. 11 shows the role of TMB in cage formation. TEM images at different magnifications of silica nanoparticles that were synthesized with (a) and without (b) TMB. Nanoparticles synthesized without TMB (b) exhibited stronger contrast at the particle center as compared to the inorganic nanocages (a), suggesting that these particles did not exhibit hollow cage-like structures but instead were conventional mesoporous silica nanoparticles with relatively small particle sizes (b).

FIG. 12 shows optical characterization of gold and silver based synthesis solutions. a, Survey of the gold based synthesis showing the absorption profile of solutions after the successive additions of HAuCl₄, THPC, one day after the addition of K₂CO₃, and compared to the same concentration of HAuCl₄ added to the equivalent water/ethanol solution but without any CTAB or TMB. b, Absorption profile of a solution obtained from the silver synthesis 6 hours after the addition of K₂CO₃.

FIG. 13 shows a high resolution TEM image of single cage-like gold nanoparticle. The gold particle exhibited lattice fringes with a spacing of 2.3 Å, consistent with the lattice spacing between (111) planes of gold (JCPDS no. 04-0784).

FIG. 14 shows size analysis of silicages. Particle size distribution of PEGylated silicages obtained by manually analyzing 450 silicages using a set of TEM images. The average cage diameter is about 11.5 nm, while more than 98% of the silicages are within the size range of ±3 nm.

FIG. 15 shows functionalization of silicages. a to c, UV-Vis absorbance spectrum (a), deconvolution of UV-Vis absorbance spectrum (b), and FCS correlation curve (c) of silicages with ATTO647N fluorescence dyes encapsulated in silica and cyclic arginine-glycine-aspartic acid (cRGDY) cancer targeting peptides attached on outer cage surface. d to f, UV-Vis absorbance spectrum (d), deconvolution of UV-Vis absorbance spectrum (e), and FCS correlation curve (f) of silicages with ATTO647N fluorescence dyes encapsulated in silica and deferoxamine (DFO) chelators attached on outer cage surface. The deconvolution of UV-Vis spectra was obtained by fitting the spectra with a linear combination of the UV-Vis spectra of individual functional groups that were attached to the silicages. The numbers of dyes and functional ligands per silicage were calculated via dividing the concentrations of individual functional groups, obtained from UV-Vis deconvolution, by the concentration of fluorescent PEGylated silicages, obtained from FCS measurements, respectively. The average particle diameters were obtained by FCS measurements.

FIG. 16 shows an example of a synthesis condition diagram, including rings, cages, silica nanoparticles, and single-pore mesoporous silica nanoparticles. The synthesis is conducted using an aqueous synthesis approach. Via tuning the regent concentrations of, as well as the molar ratio between, the surfactant, oil, and silane precursors, the geometry of the forming nanoparticles can be controlled.

FIG. 17 shows four inorganic (silica) nanoparticle topologies that were studied. Illustration of silica sphere (a), hollow bead (b), cage (c), and ring (d) topologies, together with representative EM images (e), (g), and (h), respectively. Insets in (f), (g), and (h) show individual particles, including TEM (left), and cryo-EM (right) images in (g) of the two most common projections of the dodecahedral cage, i.e., the two-fold (top) and five-fold (bottom) projections, as well as in (h) of rings lying down, and edge-on from TEM (left), and cryo-EM (right), respectively (scale bars 10 nm).

FIG. 18 shows in-vivo and ex-vivo studies of different sized spherical silica dots in mice. (a) MIP images of i.v.-injected 5.2, 6.9, and 7.8 nm FCS sized ⁸⁹Zr-labeled spherical silica nanoparticles over a 1 week period demonstrating hepatic uptake values of 1.8, 4.4, and 6.5% ID/g, respectively, (n=1 mouse/particle size). (b) Biodistribution studies for 5.2 (orange) and 7.8 nm (green) FCS sized spherical nanoparticles (n=3 mice/particle size, p<0.001) 1 week after i. v. injection. (c) Metabolic cage studies (n=3 mice/particle size) with 5.2 and 7.8 nm FCS sized spherical nanoparticles showing renal (yellow) and hepatic (brown) clearance, along with the remaining carcass (grey) activity, 1 week after i.v. injection (p<0.001). (d) Time-dependent renal/hepatic clearance levels for these same cohorts over a 6 to 168 hour period (7 days) as a function of spherical particle size (cumulative urinary clearance p<0.001, rate of accumulation p=0.017). Error bars are calculated from the standard deviation of n=3 mice for each experiment.

FIG. 19 shows in-vivo and ex-vivo murine studies of inorganic NPs with four different topologies. (a) MIP images of NPs with silica core diameters, as determined by TEM, of 7.3 nm (spheres), 10.8 nm (hollow beads), 12.3 nm (cages), and 12.1 nm (rings) at 1, ˜24, ˜48 hours, and 1-week time points after i.v. injection showing liver uptake of 6.5, 15.7, 4.1, and 2.1% ID/g, respectively, at the final 1-week time point (n=1 mouse/topology). (b) Biodistribution for spherical (orange), hollow bead (green), cage (purple), and ring (yellow) particles at 1-week time point after i.v. injection (n=3 mice/topology, p<0.001). (c) Metabolic cage studies performed on mice for each of the four different inorganic NPs (n=3 mice/topology) showing urinary (yellow) and fecal (brown) clearance along with the remaining activity in the carcass (grey) at the 1-week time point after i.v. injection (p<0.0001). (d) Time-dependent renal/hepatic clearance levels measured over a 6 to 168 hour p.i. time period (7 days) for the four topologies studied (cumulative urinary clearance p<0.0001, rate of accumulation p=0.0001). Error bars are calculated from the standard deviation of n=3 mice for each experiment.

FIG. 20 shows biodistribution studies of 12.1 nm sized (TEM) rings and liver uptake analysis for all topologies studied. (a) Blood time-activity curve indicating a blood circulation half-life, t_{1/2}, of 17.8 hours for 12.1 nm rings (n=3). (b) Time-dependent biodistribution studies (n=3) of 12.1 nm silica rings up to 1 week after i.v. injection, inset is the illustration of the onset of ring deformation enabling renal clearance and low RES uptake. Error bars are calculated from the standard deviation of n=3 mice for each experiment. (c) Dependence of liver uptake 1 week after i. v. injection (from FIGS. 18b and 19b) on TEM diameter and (d) on diffusivity, of particles with different topologies, as indicated. Inset in (d) shows the linear relationship between liver uptake and equivalent hydrodynamic diameter (Methods), derived from the diffusion coefficients, independent of particle topology (linear fit is shown as black dashed line, R²=0.979). The color code in (d) is the same as in (c).

FIG. 21 shows comprehensive characterization of particles with different topologies. Characterization of spherical dot (a-c), hollow bead (d-f), cage (g-i), and ring (j-l) particles. (a, d, g, j) FCS correlation curves with their fits for hydrodynamic sizes. (b, e, h, k) Deconvolution of the UV-vis spectra for the calculation of numbers of dyes and radiolabel chelators per particle. (c, f, i, l) GPC chromatograms for purified nanoparticles showing single peaks in all cases. GPC peak position in time does not directly correlate with size as shifts may reflect GPC configuration changes (e.g. new columns) over time (not all GPCs were taken on the same day). (m) Results of TEM size analyses (averaged over 100 particles) for spherical dot, hollow bead, cage, and ring samples.

FIG. 22 shows TEM images and tilt series of hollow beads. (a) TEM image of a hollow bead sample, with illustrations of particle topology on the right. (b) TEM images of a tilt series taken for a hollow bead sample from 0° to 45° angles. (c) Zoom-in images of individual hollow beads taken from regions highlighted by squares in the images shown in (b).

FIG. 23 shows zeta potential measurement of different topologies. Zeta potential distribution of different topologies (a), for which each sample was measured three times and the results were then averaged (b).

FIG. 24 shows comprehensive characterization of spherical dots with different sizes. Characterization of small-sized (a-c), medium-sized (d-f), and large-sized (g-i) spherical dots. (a, d, g) FCS correlation curves with their fits for hydrodynamic sizes. (b, e, h) Preparative scale GPC chromatograms for purified nanoparticles. (c, f, i) Deconvolution of the UV-vis spectra for the calculation of numbers of dyes and radiolabel chelators per particle.

FIG. 25 in-vivo and ex-vivo studies with 13.5 nm diameter silica rings. (a) TEM image (left) and illustration (right) of silica nanorings with 13.5±1.5 nm average TEM diameter (from 150 particles). (b) Biodistribution study (n=1) for the same rings as in (a) at 1 week time point after i.v. injection. (c) MIP images of the same rings as in (a) at 0.5, 24, 48, 72 hours, and 1-week time points after i. v. injection showing 2.6% ID/g liver uptake at the final time point of 1 week.

FIG. 26 shows TEM images of intact inorganic NPs in murine biological specimens, i.e. after urinary excretion. (a,b) Averaged and original TEM images (n=7) (Methods) of cages (a) and rings (b) in the urinary samples collected from murine bladders (n=2) at 2 hour post i.v. injection. For each particle, a series of TEM pictures were acquired (insets), and the results were averaged using maximum intensity (left) to improve signal-to-noise ratios. Scale bar is 20 nm.

FIG. 27 shows model calculation showing how ring stiffness depends on the radius, r, of the torus cross section. The left side shows a ring that has been flattened by applying bending moments, M, at one end. The moments, M, lead to a curvature, κ, at the ends of the ring. Approximating this curvature as constant, the relation between M and κ is as shown on the right for simple bending for the case that the ring cross section (i.e. not the radius, R, of the overall ring) is circular with radius r. Since the relation scales linearly with Young's modulus, E, one finds the difference in r that would be needed to reduce the moment, M, by an order of magnitude, i.e. M₂/M₁=0.1, at the same curvature, κ, is only a factor of 0.56. That is, the stiffness of the ring can be dramatically reduced by making it thinner without changing its modulus, E. The relation between moment, M, and curvature, κ, goes as the fourth power of the radius, r. That means, the bending moment is exquisitely sensitive to the thickness of the ring.

FIG. 28 shows dependence of spleen uptake on physical particle size and particle diffusivity. (a) Dependence of spleen uptake 1 week after i.v. injection (from FIGS. 2b and 3b) on TEM diameter and (b) on diffusivity, of particles with different topologies, as indicated in (a). Inset in (b) shows the linear relationship between spleen uptake and equivalent hydrodynamic diameter (Methods), derived from the diffusion coefficients, independent of particle topology (linear fit is shown as black dashed line, R²=0.849). The color code in (b) is the same as in (a).

FIG. 29 shows HPLC stability study of cages and rings in mouse and human serum. HPLC chromatograms of rings (a) and cages (b) after incubation in mouse (left panel) and human (right panel) serum for up to 5 days. Peak shapes and positions in HPLC elugrams remained unchanged, indicating the high stability of both topologies in serum and corroborating the notion that the elevated sizes measured for these topologies in FCS may result from smaller serum proteins hovering on the inside of these particles rather than from their physical adsorption. Experiments were performed on materials after storage in a refrigerator at 4° C. for about a year.

### DETAILED DESCRIPTION OF THE DISCLOSURE

Although claimed subject matter is described in terms of certain examples, other examples, including examples that do not provide all of the benefits and features set forth herein, are also within the scope of this disclosure. Various structural, logical, and process step changes may be made without departing from the scope of the disclosure.

Ranges of values are disclosed herein. The ranges set out a lower limit value and an upper limit value. Unless otherwise stated, the ranges include all values to the magnitude of the smallest value (either lower limit value or upper limit value) and ranges between the values of the stated range.

“About” as used herein refers to values within 5% of a base value.

The present disclosure provides inorganic nanocages. The present disclosure also provides methods of making and using the inorganic nanocages.

As used herein, unless otherwise indicated, the term “group” refers to a chemical entity that is monovalent (i.e., has one terminus that can be covalently bonded to other chemical species), divalent, or polyvalent (i.e., has two or more termini that can be covalently bonded to other chemical species). Examples of groups include, but are not limited to:

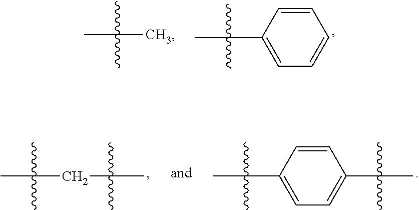

As used herein, unless otherwise indicated, the term “alkyl group” refers to branched or unbranched saturated hydrocarbon groups. Examples of alkyl groups include, but are not limited to, methyl groups, ethyl groups, propyl groups, butyl groups, isopropyl groups, tert-butyl groups, and the like. For example, the alkyl group can be a C₁ to C₁₈, alkyl group including all integer numbers of carbons and ranges of numbers of carbons therebetween (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈). The alkyl group can be unsubstituted or substituted with one or more substituent(s). Examples of substituents include, but are not limited to, various substituents such as, for example, halogens (—F, —Cl, —Br, and —I), aliphatic groups (e.g., alkyl groups, alkenyl groups, alkynyl groups), aryl groups, alkoxide groups, amine groups, carboxylate groups, carboxylic acids, ether groups, alcohol groups, alkyne groups (e.g., acetylenyl groups), and the like, and combinations thereof. An alkyl group may be part of an alkoxy group.

There are a number of important ramifications that derive from our inorganic nanocage discovery. The chemical and practical value of the polyhedral structures are considered to be extremely high. Considering the high versatility of, for example, silica, aluminosilicate, transition metal, and transition metal oxide surface chemistry, and the ability to distinguish cage inside and outside via micelle directed synthesis, one can readily conceive cage derivatives of many kinds, which may exhibit unusual properties and be useful in applications ranging from catalysis to drug delivery. For example, based on recent successes in clinical translation of ultrasmall fluorescent silica nanoparticles with similar particle size and surface properties, a whole range of novel diagnostic and therapeutic probes with drugs, which may be toxic drugs, hidden in the inside of the cages can be envisaged.

In an aspect, the present disclosure provides methods of making inorganic nanocages. A method may be based on self-assembly of inorganic nanocages.

Inorganic nanocages may be produced through self-assembly. Briefly, under the optimized synthesis conditions, inorganics self-assemble into the highly-symmetric cage structures on the surface of self-assembled surfactant micelles. The surfactant micelles may be structure directing. To trigger this unique self-assembly, a few important mechanisms have been introduced to the synthesis:
i) hydrophobic reagents, such as, for example, TMB, are first encapsulated inside the surfactant micelles, to increase micelle deformability, facilitating the cage formation;
ii) reaction kinetics of the inorganics is optimized by adjusting reaction conditions to just the right point, that primary inorganic particles can quickly form in solution to self-assemble on micelle surface. At the same time, the condensation of inorganics is quickly terminated to prevent further growth of the inorganic nanocages. Thus, the very early formation stage of mesoporous silica can be quenched, resulting in the inorganic nanocages;
iii) water is used as the reaction media, and thus hydrophobicity/hydrophilicity and electrostatic interactions can simultaneously take effect to trigger the unique self-assembly. The self-assembled cage structure shall be a result of the fine balance between these different interactions among the assembling blocks.

While one example of the synthesis procedures is described below, the range of reagent concentrations that can be used are summarized in Table 1.

TABLE 1
Reaction Parameters.

| | Concentration (mM) | | | |
|---|---|---|---|---|
| | Speci- | | | |
| | fica- | | | |
| | tion in | | | |
| | manu- | Lower | Upper | |
| Reagents | facture | Limit | Limit | Comments |

Surfactant concentrations used in all synthesis

| | | | | |
|---|---|---|---|---|
| CTAB | 34.3 | 5.5 | 82.1 | The higher the CTAB |
| | | | | concentration, the thinner |
| | | | | the arms of the cages. |
| TMB | 71.9 | 18 | 215.6 | The higher the TMB |
| | | | | concentrations, the more |
| | | | | complex the cages are. |
| | | | | e.g. moving from tetrahedral |
| | | | | to buckyball-like cages. |

Additional reagents for silica nanocages

| | | | | |
|---|---|---|---|---|
| NH₃—H₂O | 2 | 0 | 10 | The higher the NH₃—H₂O |
| | | | | concentration, the bigger |
| | | | | the cages. |
| TMOS | 67.2 | 6.7 | 201.6 | The higher the TMOS |
| | | | | concentration, the bigger |
| | | | | the cages. |

Additional reagents for gold nanocages

| | | | | |
|---|---|---|---|---|
| Ethanol | 856.3 | 85.6 | 4281.5 | NA |
| THPC | 0.14 | 0.01 | 0.6 | |
| HAuCl₄•3H₂O | 0.1 | 0.01 | 0.5 | |

Additional reagents for silver nanocages

| | | | | |
|---|---|---|---|---|
| Ethanol | 856.3 | 85.6 | 4281.5 | NA |
| THPC | 0.14 | 0.01 | 0.6 | |
| AgNO₃ | 0.1 | 0.01 | 0.5 | |

Additional reagents for vanadium oxide nanocages

| | | | | |
|---|---|---|---|---|
| Vanadium | 53 | 10.6 | 264.9 | NA |
| Oxytriiso- | | | | |
| propoxide | | | | |
| DMSO | 352 | 70.4 | 1759.9 | |

A method of making inorganic nanocages (e.g., non-metal oxide nanocages, transition metal nanocages, and transition metal oxide nanocages) may comprise forming a reaction mixture comprising one or more precursor(s); one or more surfactant(s) (e.g., surfactant(s) including positively charged groups or surfactant(s) including negatively charged groups); one or more pore expander(s) (e.g., a hydrophobic pore expander(s)); and holding the reaction mixture at a time (e.g., t¹) and/or temperature (e.g., T¹), whereby inorganic nanocages having an average size of a longest dimension (e.g., diameter) less than 30 nm are formed; and optionally, adding a terminating agent (which may be a capping agent) and/or a reductant (which may be a capping agent) to the reaction mixture.

A reaction mixture can comprise various precursors. A reaction mixture may comprise combinations of precursors. A precursor may be a non-metal oxide precursor, a metal precursor, a transition metal oxide precursor, a transition metal precursor, or a combination thereof. A transition metal precursor may be a noble metal precursor.

A non-metal oxide precursor may be a silica precursor. A metal precursor, such as, for example, one or more aluminum oxide precursor(s) may be mixed with one or more silica-generating sol-gel precursor(s) (e.g., silica precursors). A silicon alkoxide (e.g., tetraalkoxysilane, alkyltrialkoxysilane, functionalized non-metal oxide precursor, and the like) may have a plurality of alkoxy groups and the alkyl group of each of the alkoxy groups may independently be a C₁ to C₄ alkyl group and, optionally one or more alkyl group(s) directly bonded to the non-metal (e.g., silicon), where the alkyl group(s) may independently be a C₁ to C₆ alkyl group. Non-limiting examples of silica precursors include tetraalkoxysilanes (e.g., tetramethylorthosilicate (TMOS), tetraethylorthosilicate (TEOS), tetrapropylorthosilicate (TPOS), and the like), alkyltrialkoxysilanes (e.g., methyltrimethylorthosilicate), functionalized non-metal oxide precursors (e.g., (3-aminopropyl)triethoxysilane (APTES), (3-aminopropyl)trimethoxysilane (APTMS), (3-mercaptopropyl)trimethoxysilane (MPTMS), and the like and combinations thereof), and the like, and combinations thereof. It may be desirable that at least one of the non-metal precursors is TMOS (or at least one of the precursors) or the only non-metal precursor (or the only precursor) is TMOS. A functionalized non-metal precursor (e.g., a functionalized silica precursor) may be 0.1 to 20 mol % (of the total precursors).

A silica precursor may be a functionalized non-metal oxide precursor. A silica precursor may comprise one or more functional group(s) (e.g., one or more functional group(s) described herein). In non-limiting examples, a non-metal oxide precursor comprises a fluorescent dye (e.g., is a dye-silane conjugate, such as, for example, ATTO647N-silane) and/or a theranostic functional moiety (e.g., is a theranostic functional moiety-silane conjugate, such as, for example, DFO-silane) or a peptide (e.g., is a peptide-silane conjugate, such as, for example, cRGDY-silane). In other non-limiting examples, a non-metal oxide precursor comprises one or more iodide atom(s).

When non-metal precursors such as, for example, silica precursors are used, a reaction mixture may also comprise one or more aluminum oxide precursor(s). In this case, an aluminosilicate nanocage may be formed.

Various aluminum oxide precursors may be used. Combinations of aluminum oxide precursors may be used. An aluminum oxide precursor may be an aluminum-containing sol-gel precursor. Combinations of aluminum oxide precursors may be used. Non-limiting examples of aluminum oxide precursors include aluminum alkoxides, and the like, and combinations thereof. An aluminum alkoxide may have a plurality of alkoxy groups and the alkyl group of each of the alkoxy groups may be a C₁ to C₄ alkyl group. Non-limiting examples of aluminum alkoxides include aluminum butoxides (e.g., aluminum-tri-sec-butoxide), and the like, and combinations.

A precursor may be a transition metal precursor. Combinations of transition metal precursors may be used. The transition metal precursors may be used to form transition metal nanocages or transition metal oxide nanocages. In an example, a noble metal precursor may be used to form a noble metal nanocage Non-limiting examples of transition metal precursors include transition metal salts, transition metal alkoxides, transition metal coordination complexes organometallic compounds, and combinations thereof.

A transition metal precursor may form a transition metal or a transition metal oxide during the formation of the inorganic nanocage. Generally, early transition metals are more susceptible to oxidation and form transition metal oxide nanocages and late transition metals are less susceptible to oxidation and form transition metal nanocages. As illustrative examples, vanadium precursors, titanium precursors, niobium precursors, copper precursors, nickel precursors, zirconium precursors, tantalum precursors, hafnium precursors, and combinations thereof are used to form transition metal nanocages. As additional illustrative examples, gold precursors, silver precursors, platinum precursors, palladium precursors, rhodium precursors, and combinations thereof are used to form transition metal nanocages. A noble metal precursor may form a noble metal nanocage

Non-limiting examples of transition metal precursors that may form transition metal nanocages include transition metal salts, transition metal coordination complexes, and combinations thereof. Non-limiting examples of transition metal salts include gold salts (e.g., gold chlorides, gold chloride hydrates, and the like, combinations thereof), silver salts (e.g., silver halides, silver nitrates, and the like, and combinations thereof), platinum salts (e.g. potassium tetrachloroplatinate(II), palladium salts (e.g. sodium tetrachloropalladate(II)), and the like, and combinations thereof. Non-limiting examples of transition metal coordination complexes include gold coordination complexes (e.g., chloro(triphenylphosphine)gold(I)).

A transition metal precursor or transition metal precursors may be used to make transition metal oxide nanocages. A transition metal precursor may form a transition metal oxide during the formation of the inorganic nanocage. Generally, early transition metals are more susceptible to oxidation and formation of metal oxide nanocages than late transition metals. As illustrative examples, an iron precursor, a titanium precursor, or a niobium precursor can form metal oxide nanocages. A transition metal precursor or transition metal precursors may be used to form transition metal nanocages by reduction of the oxide cages formed first. Inversely, a transition metal may be used to form a transition metal cage first, which is subsequently oxidized into the transition metal oxide nanocage. As illustrative examples, a copper precursor, or a nickel precursor may be used to form a transition metal nanocage first, which can subsequently be converted via oxidation in the corresponding transition metal oxide nanocage.

Non-limiting examples of transition metal precursors that may form transition metal oxide nanocages include transition metal alkoxides, transition metal salts, transition metal coordination complexes, and combinations thereof. Non-limiting examples of transition metal alkoxides include vanadium alkoxides (e.g., vanadium oxytriisopropoxide), titanium alkoxides (e.g., titanium isopropoxide), niobium alkoxides (e.g., niobium(V) ethoxide), tantalum alkoxides (e.g. tantalum(V) ethoxide), hafnium alkoxides (e.g. hafnium(IV) tert-butoxide), zirconium alkoxides (zirconium(IV) propoxide), and combinations thereof. Non-limiting examples of transition metal salts include zirconium salts (e.g., zirconium(IV) sulfate and hydrates thereof), iron salts (e.g. iron(II) perchlorate hydrate, iron(III) nitrate nonahydrate), and combination of thereof. Non-limiting examples of transition metal coordination complexes include iron coordination complexes (e.g. iron(III) acetylacetonate).

A reaction mixture can comprise various surfactants. A reaction mixture may comprise combinations of surfactants. A surfactant may be a cationic surfactant, which may form a micelle with a positive surface charge. A surfactant may be an anionic surfactant, which may form a micelle with a negative charge.

Without intending to be bound by any particular theory, it is considered that the precursor(s) form clusters (e.g., clusters having a size, e.g., longest dimension 10 nm or less or about 2 nm) and the clusters are electrostatically attracted to a micelle surface and selectively deposit on one or more surface(s) of the micelle forming an inorganic nanocage. The clusters may be referred to as primary clusters. The clusters may be non-metal clusters (e.g., silica clusters, aluminosilicate clusters, and the like), transition metal oxide clusters (e.g., transition metal oxide clusters), and transition metal clusters (e.g., gold clusters, silver clusters, platinum clusters, palladium clusters, rhodium clusters, mixed metal clusters, and the like). The clusters may comprise a plurality of —O-NM- groups, where NM is a non-metal such as, for example, a silicon atom or the like, or a combination of non-metal atoms, a plurality of —O-TM-groups, where TM is transition metal (e.g., titanium atoms, iron atoms, niobium atoms, vanadium atoms, zirconium atoms, hafnium atoms, tantalum atoms, copper atoms, nickel atoms, and the like, and combinations thereof), or a plurality of transition metal atoms (e.g., gold atoms, silver atoms, platinum atoms, palladium atoms, rhodium atoms, and the like, and combinations thereof). It is desirable that the precursor(s) form clusters having a charge opposite that of the micelle. The pH of the reaction mixture may be adjusted to form micelles and/or clusters with a desired charge.

A cationic surfactant may be a C₁₀ to C₁₈ alkyltrimethylammonium halide. Non-limiting examples of C₁₀ to C₁₀ alkyltrimethylammonium halides include cetyltrimethylammonium bromide (CTAB), decyltrimethylammonium bromide (CioTAB), dodecyltrimethylammonium bromide (C₁₂TAB), myristyltrimethylammonium bromide (C₁₄TAB), octadecyltrimethylammonium bromide (C₁₈TAB), and the like, and combinations thereof.

An anionic surfactant may be an alkyl sulfate. Non-limiting examples of anionic surfactants include sodium dodecyl sulfate (SDS), N-myristoyl-L-glutamic acid (C14GluA), and the like, and combinations thereof.

Various amounts of surfactant(s) can be used. The surfactant(s) may be present in a reaction mixture at a concentration of 1 mg/mL to 50 mg/mL, including all integer mg values and ranges therebetween.

Various pore expanders can be used. Combinations of pore expanders may be used. A pore expander is a hydrophobic molecule. A pore expander may be disposed in a surfactant micelle (e.g., disposed in the center or in about the center of a surfactant micelle). A pore expander may be referred to as an oil. A pore expander can provide micelles that are larger than micelles formed using the same surfactant(s) in the absence of that pore expander.

A pore expander may be an alkylated benzene (e.g., a mono-, di-, or trialkylated benzene. The alkyl group(s) of the alkylated benzenes may independently be C₁ to C₆ alkyl group(s) (e.g., C₁, C₂, C₃, C₄, C₅, or C₆ groups(s)). Nonlimiting examples of alkylated benzenes include 1,2,4-trimethylbenzene (TMB), toluene, and the like. A pore expander may be a polymer monomer. Non-limiting examples of polymer monomers include stryrenes, alkylstyrenes (e.g., methyl styrene, and the like). The alkyl group(s) of the alkylstyrenese may be C₁ to C₆ alkyl group(s) (e.g., C₁, C₂, C₃, C₄, C₅, or C₆ groups(s)). A pore expander may be a hydrophobic solvent. Non-limiting examples of hydrophobic solvents include alkanes (e.g., hexane and the like), cycloalkanes (e.g., cyclohexane and the like), benzene, alkylated benzene (e.g., toluene and the like), chlorinated alkanes (e.g., chloroform and the like)), and the like, and combinations thereof.

Various amounts of pore expander(s) can be used. The pore expander(s) may be present in a reaction mixture at a concentration of 3 mg/mL to 100 mg/mL, including all integer mg values and ranges therebetween.

The surfactant(s) and pore expander(s) can be used in various ratios. The surfactant(s) and pore expander(s) may be present in a reaction mixture at molar ratio of from 1:100 to 10:1, including all 0.1 ratio values and ranges therebetween.

A reaction can be carried out for various times and/or temperatures. The reaction time may be 1 minute to 48 hours and/or the reaction temperature may be room temperature to 95° C. A reaction mixture may be formed by combining the surfactant(s), pore expanding molecule(s), and, solvent(s), if present and holding this mixture for a selected time (e.g., up to 24 hours) and temperature and subsequently adding the precursor(s).

A terminating agent may be used to stop the formation of an inorganic nanocage. A terminating agent may also be a capping agent. Combinations of terminating agents may be used. Non-limiting examples of terminating agents include PEG-silanes, which may be functionalized as described herein. In the case of silica nanocages or aluminosilicate nanocages, it may be desirable to use PEG-silane(s) as terminating agents.

PEGylation of at least a portion of a surface (e.g., an exterior surface, an interior surface, or a combination thereof) or all of the surfaces of an inorganic nanocage, which may be used to terminate and/or functionalize an inorganic nanocage, may be carried out at a variety of times and temperatures. For example, in the case of silica inorganic nanocages, PEGylation can be carried out by contacting the inorganic nanocages at room temperature up to 100° C. for 0.5 minutes to 48 hours (e.g., overnight). For example, in the case of silica or aluminosilicate inorganic nanocages the temperature is 80° C. overnight.

The chain length of the PEG moiety of the PEG-silane (i.e., the molecular weight of the PEG moiety) can be tuned from 3 to 24 ethylene glycol monomers (e.g., 3 to 6, 3 to 9, 6 to 9, 8 to 12, or 8 to 24 ethylene glycol monomers). The PEG chain length of PEG-silane can be selected to tune the thickness of the PEG layer surrounding the inorganic nanocages and the pharmaceutical kinetics profiles of the PEGylated inorganic nanocages. The PEG chain length of ligand-functionalized PEG-silane can be used to tune the accessibility of the ligand groups on the surface of the PEG layer of the inorganic nanocages resulting in varying binding and targeting performance.

PEG-silane conjugates may comprise a ligand. The ligand is covalently bound to the PEG moiety of the PEG-silane conjugates (e.g., via the hydroxy terminus of the PEG-silane conjugates). The ligand can be conjugated to a terminus of the PEG moiety opposite the terminus conjugated to the silane moiety. The PEG-silane conjugate can be formed using a heterobifunctional PEG compound (e.g., maleimido-functionalized heterobifunctional PEGs, NHS ester-functionalized heterobifunctional PEGs, amine-functionalized heterobifunctional PEGs, thiol-functionalized heterobifunctional PEGs, etc.). Examples of suitable ligands include, but are not limited to, linear or cyclic peptides (natural or synthetic), fluorescent dyes, absorbing dyes, sensing molecules, ligands comprising a radio label (e.g., ¹²⁴I, ¹³¹I, ²²⁵Ac, ¹⁷⁷Lu, and the like, and combinations thereof), antibodies, nucleic acids (e.g., DNA, RNA, and the like), ligands comprising a reactive group (e.g., a reactive group that can be conjugated to a molecule such a drug molecule, gefitinib, and the like), including amines, carboxylic acids, esters (e.g., activated esters), azides, alkenes, alkynes, and the like.

For example, PEG-silane conjugate comprising a ligand is added in addition to PEG-silane. In this case, inorganic nanocages surface functionalized with polyethylene glycol groups and polyethylene groups comprising a ligand or inorganic nanocages surface functionalized with polyethylene glycol groups and polyethylene groups comprising a ligand are formed. The conversion percentage of ligand-functionalized or reactive group-functionalized PEG-silane is 40% to 100% and the number of ligand-functionalized PEG-silane precursors reacted with each particle is 3 to 50,000.

For example, before or after (e.g., 20 seconds to 5 minutes before or after) the PEG-silane conjugate is added, a PEG-silane conjugate comprising a ligand (e.g., at concentration between 0.05 mM and 2.5 mM) is added at room temperature to the reaction mixture comprising the inorganic nanocages, respectively. The resulting reaction mixture is held at a time and temperature (e.g., 0.5 minutes to 48 hours at room temperature up to 100° C.), where at least a portion of the PEG-silane conjugate molecules are adsorbed on at least a portion of the surface of the inorganic nanocages. Subsequently, the reaction mixture is heated at a time and temperature (e.g., 0.5 minutes to 48 hours at 40° C. to 100° C.), where inorganic nanocages surface functionalized with polyethylene glycol groups comprising ligand inorganic nanocages surface functionalized with polyethylene glycol groups comprising a ligand are formed. Optionally, subsequently adding at room temperature to the resulting reaction mixture comprising inorganic nanocages surface functionalized with polyethylene glycol groups comprising a ligand a PEG-silane conjugate (the concentration of PEG-silane no ligand is between 10 mM and 75 mM) (e.g., PEG-silane conjugate dissolved in a polar aprotic solvent such as, for example, DMSO or DMF), holding the resulting reaction mixture at a time and temperature (e.g., 0.5 minutes to 48 hours at room temperature to 100° C.) (whereby at least a portion of the PEG-silane conjugate molecules are adsorbed on at least a portion of the surface of the inorganic nanocages surface functionalized with polyethylene glycol groups comprising a ligand a PEG-silane conjugate, and heating the resulting mixture from at a time and temperature (e.g., 0.5 minutes to 48 hours at 40° C. to 100° C.), whereby inorganic nanocages surface functionalized with polyethylene glycol groups and polyethylene glycol groups comprising a ligand are formed.

In another example, at least a portion of or all of the PEG-silane has a reactive group on a terminus of the PEG moiety opposite the terminus conjugated to the silane moiety of the PEG-silane conjugate (is formed from a heterobifunctional PEG compound) and after formation of the inorganic nanocages surface functionalized with polyethylene glycol groups having a reactive group. Optionally, polyethylene glycol groups are reacted with a second ligand (which can be the same or different than the ligand of the inorganic nanocages surface functionalized with polyethylene glycol groups and polyethylene glycol group comprising a ligand) functionalized with a second reactive group (which can be the same or different than the reactive group of the inorganic nanocages surface functionalized with polyethylene glycol groups and polyethylene glycol group comprising a ligand) thereby forming inorganic nanocages surface functionalized with polyethylene groups functionalized with a second ligand and, optionally, polyethylene glycol groups.

In another example, at least a portion of or all of the PEG-silane has a reactive group on a terminus of the PEG moiety opposite the terminus conjugated to the silane moiety of the PEG-silane conjugate (is formed from a heterobifunctional PEG compound) and after formation of the inorganic nanocages surface functionalized with polyethylene glycol groups and, optionally having a reactive group, and, optionally, polyethylene glycol groups, inorganic nanocages surface functionalized with polyethylene glycol groups having a reactive group, and, optionally, polyethylene glycol groups, are reacted with a second ligand (which can be the same or different than the ligand of the inorganic nanocages surface functionalized with polyethylene glycol groups and polyethylene glycol group comprising a ligand) functionalized with a second reactive group (which can be the same or different than the reactive group of the inorganic nanocages surface functionalized with polyethylene glycol groups and polyethylene glycol group comprising a ligand) thereby forming inorganic nanocages surface functionalized with polyethylene groups functionalized with a second ligand and, optionally, polyethylene glycol groups, where at least a portion of the PEG-silane has a reactive group on a terminus of the PEG moiety opposite the terminus conjugated to the silane moiety of the PEG-silane conjugate (is formed from a heterobifunctional PEG compound) and after formation of the inorganic nanocages surface functionalized with polyethylene glycol groups having a reactive group or inorganic nanocages surface functionalized with polyethylene glycol groups having a reactive group and polyethylene glycol groups comprising a ligand are reacted with a second ligand functionalized with a reactive group (which can be the same or different than the ligand of the inorganic nanocages surface functionalized with polyethylene glycol groups and polyethylene glycol group comprising a ligand) thereby forming inorganic nanocages surface functionalized with polyethylene glycol groups and polyethylene groups functionalized with a second ligand or inorganic nanocages surface functionalized with polyethylene glycol groups comprising a ligand, or inorganic nanocages functionalized with polyethylene glycol groups and polyethylene groups comprising a ligand that is functionalized with the second ligand.

The inorganic nanocages with PEG groups functionalized with reactive groups may be further functionalized with one or more ligand(s). For example, a functionalized ligand can be reacted with a reactive group of a PEG group. Examples of suitable reaction chemistries and conditions for post-nanoparticle synthesis functionalization are known in the art.

A terminating agent may be a reducing terminating agent. The reducing terminating agent may also be a capping agent. Combinations of reducing terminating agents may be used. In the case of transition metal nanocages, without intending to be bound by any particular theory, it is considered that the reducing terminating agent reduces the transition metal precursor, caps the transition metal nanocages, and may endow the nanocages with a desirable surface charges. Non-limiting examples of reducing terminating agents include tetrakis(hydroxymethyl)phosphonium chloride (THPC), bis[tetrakis(hydroxymethyl)phosphonium] sulfate (THPS), tris(hydroxymethyl)phosphine, and the like, and combinations thereof.

A reaction mixture may comprise one or more solvent(s). In an example, a reaction mixture further comprises a solvent and the solvent is water and the pH of the reaction mixture is 5 or greater (e.g., 5-9) or 6 or greater (e.g., 6-9).

The methods may be carried out in a reaction mixture comprising an aqueous reaction medium (e.g., water). For example, the aqueous medium comprises water. Certain reactants may be added to the various reaction mixtures as solutions in a polar aprotic solvent (e.g., DMSO or DMF). In various examples, the aqueous medium does not contain organic solvents (e.g., alcohols such as C₁ to C₆ alcohols) other than polar aprotic solvents at 10% or greater, 20% or greater, or 30% or greater. In an example, the aqueous medium does not contain alcohols at 1% or greater, 2% or greater, 3% or greater, 4% or greater, or 5% or greater. In an example, the aqueous medium does not contain any detectible alcohols. For example, the reaction medium of any of the steps of any of the methods disclosed herein consists essentially of water and, optionally, a polar aprotic solvent.

At various points in the methods the pH can be adjusted to a desired value or within a desired range. The pH of the reaction mixture can be increased by addition of a base and/or lowered by addition of an acid. Non-limiting examples of bases include ammonium hydroxide (which may be desirable in the case of methods of making silica nanocages), carbonates, such as, for example, potassium carbonate, (which may be desirable in methods of making transition metal nanocages, alkali hydroxides, such as, for example, sodium hydroxide or potassium hydroxide, and the like, and combinations thereof. Non-limiting examples of suitable acids include inorganic acids (e.g. hydrochloric acid, nitric acid, sulfuric acid), organic acids (e.g. acetic acid), and the like, and combinations thereof.

In the case of aluminosilicate inorganic cage synthesis, the pH of the reaction mixture is adjusted to a pH of 1 to 4 prior to addition of the aluminum oxide precursor. After aluminosilicate nanocage formation, the pH of the solution is adjusted to a pH of 7 to 9 and, optionally, PEG with molecular weight between 100 and 1,000 g/mol, including all integer values and ranges therebetween, at concentration of 10 mM to 75 mM, including all integer mM values and ranges therebetween, is added to the reaction mixture prior to adjusting the pH of the reaction mixture to a pH of 7 to 9.

The inorganic nanocages can be functionalized. The inorganic nanocages can be functionalized using various methods. At least a portion of a surface (e.g., at least a portion of an exterior surface and/or at least a portion of an interior surface of the inorganic nanocages may be functionalized (e.g., covalently functionalized and/or non-covalently functionalized).

The inorganic nanocages may be selectively functionalized. The functionalization may be the same for the interior surface and exterior surface of the inorganic nanocages or may be different for the interior surface and exterior surface of the inorganic nanocages. The inorganic nanocages may be selectively functionalized by functionalizing the exterior surface of the inorganic nanocages while the micelle is disposed in the interior of the inorganic nanocage and subsequently functionalizing the interior of the inorganic nanocage after removal of the micelle.

In an example, a PEGylated inorganic nanocage is reacted with one or more functionalizing precursor(s) and one or more functional group precursor(s) The reactions can be carried out in any order, so long as the inorganic nanocage is first reacted with at least one functionalizing precursor. For example, an inorganic nanocage with a single type of reactive group is reacted with one or more functional group precursor(s). In another example, an inorganic nanocage with two or more structurally and/or chemically different reactive groups (e.g., 2, 3, 4, or 5 structurally and/or chemically different reactive groups) is reacted with two or more different functional group precursors (e.g., 2, 3, 4, or 5 structurally and/or chemically different functional group precursors), where the individual reactive groups/functional group precursors may have orthogonal reactivity.

Various conjugation chemistries/reactions may be used to covalently link a functional group to the surface of an inorganic nanocage. Accordingly, a functionalizing precursor can comprise various reactive groups. Numerous suitable conjugation chemistries and reactions are known in the art. In various examples, a reactive group is one that reacts in particular conjugation chemistry or reaction known in the art and the functional group precursor comprises a complementary group of the particular conjugation chemistries/reactions known in the art.

Functionalizing precursors may comprise one or more reactive group(s) and a group (e.g., a silane group) that can react with the surface of the inorganic nanocage to form a covalent bond. The reactive group(s) can react with a functional group precursor to form a functional group that is covalently bound to the surface of the inorganic nanocage. Non-limiting examples of reactive groups include an amine group, a thiol group, a carboxylic acid group, a carboxylate group, an ester group (e.g., an activated ester group), a maleimide group, an allyl group, a terminal alkyne group, an azide group, a thiocyanate group, and the like, and combinations thereof. Examples of functionalizing precursors are known in the art and are commercially available or can be made using methods known in the art.

In various examples, a functionalizing precursor comprises a silane group that comprises one or more —Si—OH group(s) (e.g., 1, 2, or 3 Si—OH groups) and at least one reactive group (e.g., 1, 2, or 3 reactive groups). The silane group(s) and reactive group(s) may be covalently bonded via a linking group such as, for example, an alkyl group (e.g., a C₁, C₂, C₃, C₄, C₅, C₆, C₇, or C₈ alkyl group). Without intending to be bound by any particular theory, it is considered that the Si—OH group of the functionalizing precursor reacts with a surface hydroxyl group of the inorganic nanocage (e.g., a surface Si—OH group).

An inorganic nanocage or a plurality of inorganic nanocages may be reacted to form various numbers of reactive groups and/or functional groups. For example, a nanoparticle or a plurality of inorganic nanocages is reacted to form 1 to 100 reactive group(s) and/or functional group(s), including all integer number of reactive groups and ranges therebetween, (e.g., plurality of inorganic nanocages is reacted to form an average of 1 to 100 group(s) and/or functional group(s), including all integer number of reactive groups and ranges therebetween, per inorganic nanocage for a plurality of inorganic nanocages) covalently bound to the surface of the inorganic nanocage or plurality of inorganic nanocages. In various examples, an inorganic nanocage or a plurality of inorganic nanocages is reacted to form 20 to 100, 25 to 100, 30 to 100, 35 to 100, 40 to 100, or 50 to 100 group(s) and/or functional group(s) (e.g., plurality of inorganic nanocages can be reacted to form an average of 20 to 100, 25 to 100, 30 to 100, 35 to 100, 40 to 100, or 50 to 100 group(s) and/or functional group(s) covalently bound to the surface of each of the inorganic nanocages. Determining reaction conditions (e.g., reactant concentrations, reaction time, reaction temperature, and the like, or a combination thereof) to form a desired number of group(s) and/or functional group(s) is/are within the purview of one having skill in the art.

A functional group precursor may react with a reactive group of an inorganic nanocage to form a functional group covalently bound to a surface of the inorganic nanocage. A functional group precursor comprises a functional group (e.g., a dye group, chelator group, targeting group, drug group, radio label/isotope group, and the like, which may be derived from a dye molecule, chelator molecule, targeting molecule, etc.) and a group that can react with a reactive group of an inorganic nanocage. Non-limiting examples of groups that react with a reactive group include an amine group, a thiol group, a carboxylic acid group, a carboxylate group, an ester group (e.g., an activated ester group), a maleimide group, an allyl group, a terminal alkyne group, an azide group, a thiocyanate group, and combinations thereof. In various examples, a functional group precursor comprises one or more group(s) that react in a particular conjugation chemistry or reaction known in the art (e.g., the functional group precursor comprises one or more group(s), such as, for example, an azide, that is complementary to a reactive group of the nanoparticle, such as for example, a terminal alkyne, in a particular conjugation chemistry/reaction, such as, for example, click chemistry, known in the art). Examples of functional group precursors are known in the art and are commercially available or can be made using methods known in the art.

Various functional groups are known in the art. A functional group may also be referred to herein as a ligand. The functional groups have various functionality (e.g., absorbance/emission behavior such as, for example, fluorescence and phosphorescence, which can be used for imaging, sensing functionality (e.g., pH sensing, ion sensing, oxygen sensing, biomolecules sensing, temperature sensing, and the like), chelating ability, targeting ability (e.g., antibody fragments, aptamers, proteins/peptides (natural, truncated, or synthetic, and the like), nucleic acids such as, for example, DNA and RNA, and the like), diagnostic ability (e.g., radioisotopes and the like), therapeutic ability (e.g., drugs, nucleic acids and the like), and the like and combinations thereof. A functional group may have both imaging and therapeutic functionality. A functional group can be formed from a compound exhibiting functionality by derivatization of the compound using conjugation chemistry and reactions known in the art.

The functional group(s) carried by the inorganic nanocages can include diagnostic and/or therapeutic agents (e.g., radioisotopes, drugs, nucleic acids, and the like). An inorganic nanocage may comprise a combination of different functional groups.

Non-limiting examples of therapeutic agents, which may be drugs, include, but are not limited to, chemotherapeutic agents, antibiotics, antifungal agents, antiparasitic agents, antiviral agents, and combinations thereof, and groups derived therefrom. Examples of suitable drugs/agents are known in the art.

An inorganic nanocage may comprise various dyes (e.g., functional groups formed from various dyes). In various examples, the dyes are organic dyes. In an example, a dye does not comprise a metal atom. Non-limiting examples of dyes include fluorescent dyes (e.g., near infrared (NIR) dyes), phosphorescent dyes, non-fluorescent dyes (e.g., non-fluorescent dyes exhibiting less than 1% fluorescence quantum yield), fluorescent proteins (e.g., EBFP2 (variant of blue fluorescent protein), mCFP (Cyan fluorescent protein), GFP (green fluorescent protein), mCherry (variant of red fluorescent protein), iRFP720 (Near Infra-Red fluorescent protein)), and the like, and groups derived therefrom. In various examples, a dye absorbs in the UV-visible portion of the electromagnetic spectrum. In various examples, a dye has an excitation and/or emission in the near-infrared portion of the electromagnetic spectrum (e.g., 650-1700 nm).

Non-limiting examples of organic dyes include cyanine dyes (e.g., Cy5®, Cy3®, Cy5.5®, Cy7®, Cy7.5®, and the like), carborhodamine dyes (e.g., ATTO 647N (available from ATTO-TEC and Sigma Aldrich®), BODIPY dyes (e.g., BODIPY 650/665 and the like), xanthene dyes (e.g., fluorescein dyes such as, for example, fluorescein isothiocyanate (FITC), Rose Bengal, and the like), eosins (e.g. Eosin Y and the like), and rhodamines (e.g. TAN/IRA, tetramethylrhodamine (TMR), TRITC, DyLight® 633, Alexa 633, HiLyte 594, and the like), Dyomics® DY800, Dyomics® DY782 and IRDye® 800CW, and the like, and groups derived therefrom.

An inorganic nanocage may comprise various sensor groups. Non-limiting examples of sensor groups include pH sensing groups, ion sensing groups, oxygen sensing groups, biomolecule sensing groups, temperature sensing groups, and the like. Examples of suitable sensing compounds/groups are known in the art.

An inorganic nanocage can comprise various chelator groups. Non-limiting examples of chelator groups include desferoxamine (DFO), 1,4,7,10-Tetraazacyclododecane-1,4,7,10-tetraacetic acid (DOTA), 1,4,7-triazacyclononane-1,4,7-triacetic acid (NOTA), ethylenediaminetetraacetic acid (EDTA), di ethylenetriaminepentaacetic acid (DTPA), porphyrins, and the like, and groups derived therefrom. A chelator group may comprise a radioisotope. Examples of radioisotopes are described herein and are known in the art.

A radioisotope can be a functional group. A radioisotope can be a diagnostic agent and/or a therapeutic agent. For example, a radioisotope, such as for example, ¹²⁴I, is used for positron emission tomography (PET). Non-limiting examples of radioisotopes include ¹²⁴I, ¹³¹I, ²²⁵Ac, ¹⁷⁷Lu, and the like. A radioisotope may be chelated to a chelating group.

A targeting group may also be conjugated to the inorganic nanocage to allow targeted delivery of an inorganic nanocage. A targeting group can be formed from (derived from) a targeting molecule. For example, a targeting group, which is capable of binding to a cellular component (e.g., on the cell membrane or in the intracellular compartment) associated with a specific cell type, is conjugated to the inorganic nanocage. The targeting group may be a tumor marker or a molecule in a signaling pathway. The targeting group may have specific binding affinity to certain cell types, such as, for example, tumor cells. In certain examples, the targeting group may be used for guiding the inorganic nanocages to specific areas, such as, for example, liver, spleen, brain or the like. Imaging can be used to determine the location of the inorganic nanocages in an individual. Examples of targeting groups include, but are not limited to, linear and cyclic peptides (e.g., αᵥβ₃ integrin-targeting cyclic(arginine-glycine-aspartic acid, tyrosine-cysteine) peptides, c(RGDyC), and the like), antibody fragments, various DNA and RNA segments (e.g. siRNA).

As used herein, unless otherwise stated, the term “derived” refers to formation of a group by reaction of a native functional group of a compound (e.g., formation of a group via reaction of an amine of a compound and a carboxylic acid to form a group) or chemical modification of a compound to introduce a new chemically reactive group on the compound that is reacted to form a group.

The inorganic nanocages may be subjected to post-synthesis processing steps. For example, after synthesis, the solution is cooled to room temperature and then transferred into a dialysis membrane tube (e.g. a dialysis membrane tube having a Molecular Weight cut off of 10,000, which are commercially available (e.g., from Pierce)). The solution in the dialysis tube is dialyzed in a solvent mixture of DI-water, ethanol, and acetic acid at a volume ratio of between 500:500:1 to 500:500:50 (volume of solvent is 50 times more than the reaction volume, e.g. 500 mL water for a 10 mL reaction). The washing solvent may be changed every day for one to six days to extract surfactant molecules from nonage interiors and wash away remaining reagents (e.g., ammonium hydroxide, surfactant, oil, and free silane molecules). The solution in the dialysis tube may then be dialyzed in DI-water (volume of water is 200 times more than the reaction volume, e.g. 2000 mL water for a 10 mL reaction) and the water is changed every day for one to six days to wash away remaining reagents, e.g., ammonium hydroxide and free silane molecules. The particles are then filtered through a 200 nm syringe filter (Fisher Brand) to remove aggregates or dust. If desired, additional purification processes, including gel permeation chromatography and high-performance liquid chromatography, can be applied to the inorganic nanocages to further ensure the high purify of the synthesized particles (e.g., 1% or less unreacted reagents or aggregates). After any purification processes, the purified inorganic nanocages can be transferred back to deionized water if other solvent is used in the additional processes.

In a non-limiting examples, a method comprises, before or after the PEG-silane conjugate is added, if a PEG-silane is added, adding a PEG-silane conjugate comprising a ligand at room temperature to the reaction mixture, holding the resulting reaction mixture at a time (e.g., t²) and temperature (e.g., T²), subsequently heating the resulting reaction mixture at a time (e.g., t³) and temperature (e.g., T³), whereby inorganic nanocages surface functionalized with PEG groups comprising a ligand are formed.

In other non-limiting examples, at least a portion of or all of the PEG-silane has a reactive group on a terminus of the PEG group opposite the terminus conjugated to the silane group of the PEG-silane conjugate and after formation of the inorganic nanocages surface functionalized with PEG groups having a reactive group, and, optionally, PEG groups, are reacted with a second ligand functionalized with a second reactive group thereby forming inorganic nanocages surface functionalized with polyethylene groups functionalized with a second ligand and, optionally, PEG groups.

In still other examples, at least a portion of or all of the PEG-silane has a reactive group on a terminus of the PEG moiety opposite the terminus conjugated to the silane moiety of the PEG-silane conjugate and after formation of the inorganic nanocages surface functionalized with PEG groups and, optionally having a reactive group, and, optionally, PEG groups, are reacted with a second ligand functionalized with a second reactive group thereby forming inorganic nanocages surface functionalized with polyethylene groups functionalized with a second ligand and, optionally, PEG groups,

Transition metal nanocages and transition metal oxide nanocages may be functionalized. The transition metal oxide nanocages may be functionalized as described herein for non-metal nanocages (e.g., silica nanocages). The transition metal nanocages may be functionalized by reaction with amine containing ligands (e.g., PEG-amines, dodecylamine and the like), thiol containing ligands (e.g., PEG-thiols, dodecanethiol, mercaptoundecanoic acid, and the like), or phosphine containing ligands (e.g. triphenyl phosphine and the like), and the like, and combinations thereof. The ligands may comprise functional groups as described herein.

A method may comprise one or more isolation/separation process(es). Non-limiting examples of isolation/separation processes include size exclusion chromatography, high performance liquid chromatography, and gel permeation chromatography. Using one or more isolation/separation process(es) at least a portion (or all) of the inorganic nanocages are isolated from the reaction mixture (e.g., unreacted precursor(s).

The micelles and pore expander molecules may be removed from the inorganic nanocages. For example, the micelles and pore expander molecules are removed from the inorganic nanocages by dialysis. For example, after synthesis, the solution is cooled to room temperature and then transferred into a dialysis membrane tube (e.g. a dialysis membrane tube having a Molecular Weight cut off of 10,000, which are commercially available (e.g., from Pierce)). The solution in the dialysis tube is dialyzed in a solvent mixture of DI-water, ethanol, and acetic acid at a volume ratio of between 500:500:1 to 500:500:50 (volume of solvent is 50 times more than the reaction volume, e.g. 500 mL water for a 10 mL reaction). The washing solvent is changed every day for one to six days to extract surfactant molecules from nonage interiors and wash away remaining reagents e.g., ammonium hydroxide, surfactant, oil, and free silane molecules. The solution in the dialysis tube is then dialyzed in DI-water (volume of water is 200 times more than the reaction volume, e.g. 2000 mL water for a 10 mL reaction) and the water is changed every day for one to six days to wash away reagents ethanol and acetic acid.

In the case of reaction mixtures comprising polymerizable pore expander molecules, the polymerizable pore expander molecules may be polymerized to form inorganic nanocage composites. The polymerizable pore expander molecules may be polymerized by methods known in the art. For example, the polymerization can be carried out by use of a water insoluble radical initiator which generates radicals via heating or illumination with light (typically UV light) which in turn initiates the radical polymerization.

The methods can provide inorganic nanocages may have various sizes. The inorganic nanocages may have a size, e.g., a longest dimension, which may be a longest linear dimension, which may be a diameter, (e.g., an average longest linear dimension) of less than 30 nm. The inorganic nanocages may have a size, e.g., a longest dimension, which may be a longest linear dimension, which may be a diameter (e.g., average longest linear dimension) of 5 nm to less than 30 nm, 5 to 20 nm, 5 to 15 nm, or 5 to 10 nm. For example, the inorganic nanocages may have a longest linear dimension or average longest linear dimension of less than 5 nm to slightly more than 20 nm or slightly more than 10 nm. The size or average size may or may not include any surface functional groups of an inorganic nanocage. In various examples, the size or average size of all of the inorganic nanocages in a batch (inorganic nanocages formed in a single reaction) is within 5% or less of the average size, 4% of the average size, 3% or less of the average size, 2% or less of the average size, or 1% or less of the average size. For the exemplary size distributions, the inorganic nanocages may not have been subjected to any particle-size discriminating (size selection/removal) processes (e.g., filtration, dialysis, chromatography (e.g., GPC), centrifugation, etc.).

Without intending to be bound by any particular theory, it is considered that the average size of a batch (inorganic nanocages formed in a single reaction) can be selected by selecting on or more of the reaction components, ratio of two or more reaction components, reaction conditions, or the like. As an illustrative example, the size of the inorganic nanocages, in the case of silica nanocages, typically, when all other things being the same, increases when the surfactant:pore expander molar ratio decreases.

In an aspect, the present disclosure provides inorganic nanocages. The inorganic nanocages may be produced by a method of the present disclosure.

The inorganic nanocages are discrete nanoscale structures. The inorganic nanocages may be referred to nanoparticles, particles, cage-like structures, nanocages, or cages. The inorganic nanocages may have cage-like polyhedral shapes, which may have icosahedral symmetry. The inorganic nanocages comprise a plurality of polygons that form the inorganic nanocage. The polygons may all have the same shape or two or more of the polygons have different shapes. For example, the inorganic nanocages comprise the following surface polygons (where the exponent describes how often a polygon appears on the surface of the cage): 3³4³, 4⁴5⁴, 4³5⁶6³, 3³4³5⁹, 5¹² (dodecahedral) 5¹²6², 4⁶6⁸, 5¹²6³, 5¹²6⁴, 4³5⁹6²7³, 5¹²6⁸, 5¹²6²⁰ (buckyball) or the like.

The inorganic nanocages may comprise non-metal atoms in an oxidized state, metal atoms in an oxidized state (e.g., in the case of aluminosilicate nanocages), transition metal atoms in a neutral state or oxidized state, and combinations thereof. The inorganic nanocages may also comprise oxygen atoms. The inorganic nanocages may be non-metal oxide nanocages, transition metal nanocages, and transition metal oxide nanocages. Non-limiting examples of non-metal oxide nanocages include silica nanocages, which may be referred to as silicages. A non-metal oxide nanocage may also include a metal oxide such as, for example, aluminum oxide (e.g., alumina). A non-limiting example of such non-metal oxide nanocages include aluminosilicate nanocages. Non-limiting examples of transition metal nanocages include gold nanocages, silver nanocages, platinum nanocages, palladium nanocages, rhodium nanocages, and the like. Non-limiting examples of transition metal oxide nanocages include vanadium oxide nanocages, titanium oxide nanocages, niobium oxide nanocages, copper oxide nanocages, nickel oxide nanocages, zirconium oxide nanocages, tantalum oxide nanocages, hafnium oxide nanocages, and the like. A transition metal nanocage may be a noble metal nanocage comprising a transition metal that is a noble metal.

The inorganic nanocages include, in various examples, a series of desirably-symmetric (e.g., highly-symmetric) cage structures at the nano-scale (instead of atomic scale structure in molecular cages). The inorganic nanocages may exhibit highly-symmetric cage structures, including, but not limited to, dodecahedral, icosahedral, cubic, hexanol, tetrahedral, octahedral, buckyball-like cages, and the like.

Inorganic nanocages are different from, for example, biomaterials, such as cages formed from DNA, RNA, proteins, and viruses, which also may exhibit highly-symmetric structures, where the composition of the nanocages is inorganic. The inorganic nanocages may be made from, for example, a single inorganic material such as, but not limited to, silica, gold, silver, vanadium oxide, or the like. These inorganic nanocages are different from other biomaterials, such as DNA, RNA, proteins, and viruses, which also sometime exhibit highly-symmetric structures, the composition of the inorganic nanocages described here is inorganic, including, but not limited to, silica, gold, silver, and vanadium oxide. The inorganic nanocages may also be made from, for example, a mixture of inorganic materials. The mixture may have a disordered, glassy structure or may be a non-ordered alloy, or may have an ordered structure like in intermetallic materials.

Inorganic nanocages may have various sizes. The inorganic nanocages may have a size, e.g., a longest dimension, which may be a longest linear dimension, which may be a diameter, of less than 30 nm. The inorganic nanocages may have a size, e.g., a longest dimension, which may be a longest linear dimension, which may be a diameter, of 5 to less than 30 nm, 5 to 20 nm, or 5 to 15 nm. For example, the inorganic nanocages may have a size, e.g., a longest dimension, which may be a longest linear dimension, which may be a diameter, of less than 5 nm to slightly more than 20 nm or slight more than 10 nm. The size may or may not include any surface functional groups of an inorganic nanocage.

Without intending to be bound by any particular theory, it is considered that the inorganic nanostructures are flexible and can deform to pass through channels having a width smaller than the inorganic nanocage size. It is considered that inorganic nanocages having a size (e.g., longest dimension) greater than would typically allow renal clearance from an individual by the kidneys are cleared from an individual by the kidneys.

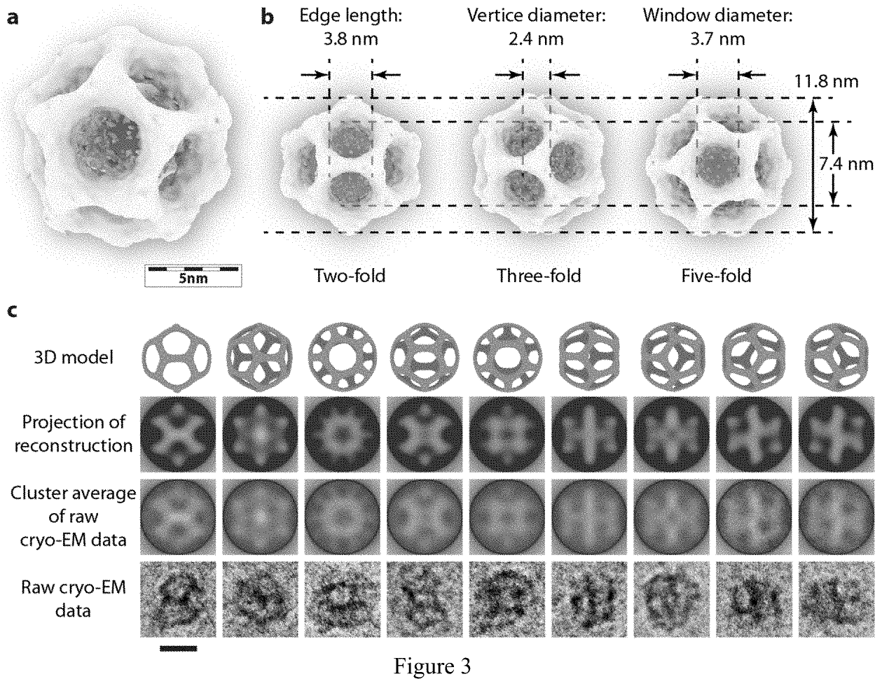

The inorganic nanocages may have several structural features. These features may include an interior, a plurality of apertures (which may be referred to as “windows” or “open windows”), arms (which may be referred to as “struts” or “edges”), and vertices. Examples of structural features are shown in FIG. 3.

The inorganic nanocages may have a fully empty interior. The apertures of the inorganic nanocage may connect the interior of the inorganic nanocage to the outside environment. That is, material from the outside environment may pass through an aperture into the interior of the inorganic nanocage. The inorganic nanocages have fully empty interior, while there are open windows on the cages connecting the inside and outside.

The point at which several arms (edges) meet is referred to as a vertice. The vertices of the inorganic nanocages may have a longest linear dimension (e.g., a diameter) about 1 to about 5 nm, including every 0.1 nm value and range therebetween. The arms connecting two nearby vertices of the inorganic nanocages may have a longest linear dimension (e.g., diameter) of less than 1 to about 3 nm or less than or equal to 1 to about 5 nm. For example, the struts of the inorganic nanocages are around 2 nm thick and only contains a few atoms across the cross-section.

An inorganic nanocage has a plurality of apertures. The apertures can have various shapes. The inorganic nanocage may have apertures having all the same shape or have apertures having two or more shapes. The apertures may independently have a size (e.g., a longest dimension in a plane defining the aperture), such as, for example, a diameter, of 1 to 10 nm, including all 0.1 nm value and ranges therebetween. The apertures may have a size of 2 to 7 nm. The apertures (i.e., windows) of the inorganic nanocages may have a longest linear dimension (e.g., a diameter) of about 1 nm to about 5 nm, including every 0.1 nm value and range therebetween. For example, in a nanocage, a portion of the vertices and a portion of the arms define a polygon and an aperture defines at least a portion of that polygon.

The size of the inorganic nanocages may be determined by both the geometry of cage structure and the composition of materials, while the aperture (i.e., window) sizes may be similar or different form the cages containing same material composition but with different structure geometries.

In an example, dodecahedral silica nanocages have an average diameter around 12 nm. In comparison, silica inorganic nanocages with the more complex geometries, such as buckyballs, are substantially bigger, while the silica nanocages with the simpler geometries, such as tetrahedral cages, are smaller.

When silica is replaced by other metallic materials (e.g. gold and silver), the size of the inorganic nanocages may be slightly reduced. When the nanocage composition is metallic, the inorganic nanocages may be crystalline.

An inorganic nanocage (e.g., the arms, vertices, and the like thereof) comprises an inorganic material matrix (e.g., silica matrix). A portion of or all the inorganic material matrix of an inorganic nanocage (e.g., the silica matrix of a silica nanocage) may be microporous. A portion or portions of or all the inorganic material matrix of an inorganic nanocage (e.g., the silica matrix of a silica nanocage) may be functionalized. Non-limiting examples of functionalization(s) are provided herein. The structural features (e.g., the arms, vertices, and the like thereof) of an inorganic nanocage (e.g., silica nanocage) may have various sizes. The individual structural features may have modulated thickness (e.g., one or more modulated dimension(s) normal to a long axis of the structural feature). In various examples, some or all of the structural features of an inorganic nanocage (e.g., silica nanocage) have modulated thickness(es). In various examples, the inorganic material matri(ces) (e.g., silica matri(ces)) of an inorganic nanocage (e.g., silica nanocage) has/have a modulated diameter/modulated diameters, a modulated radius/modulated radii, or the like.

In various examples, an inorganic material matrix (e.g., silica matrix) has/the inorganic material matrices (e.g., silica matrices) independently have a plurality of inorganic material domains (e.g., silica domains), where two domains (which may referred to as first domains) are connected by (e.g., covalently bonded by) a plurality of bonds (e.g., Si—O—Si bonds or the like) by an inorganic material matrix (e.g., silica domain) (which may be referred to as a second domain, such as, for example, a second silica domain) and this domain (e.g., second silica domain), has a dimension normal to a long axis of the silica matrix that is 50% or less (e.g., 10-50%, including all 0.1% values and ranges therebetween) than a dimension normal to a long axis of the inorganic material matrix (e.g., silica matrix) of one or both of the two domains (e.g., first domain(s)). A second domain may be referred to as a linker. In the case of a silica nanocage, the two domains (e.g., first domain(s)) may have (e.g., predominantly have) a Q3 silica structure (e.g., may comprise a plurality of Q3 bonded silicon atoms). A second domain (or linker) may predominantly have a Q2 silica structure (e.g., second domain (or linker) may comprise a plurality of linear silicon-oxygen-silicon groups (e.g., a plurality of Si—O—Si—O—Si groups arranged in a linear manner, which may be an oligomeric siloxane group or a polysiloxane group or oligomeric siloxane groups or polysiloxane groups). In various examples, the silica matrix comprises 30% or more, 40% or more, 50% or more, or 60% or more Q4 silicon atoms. In various other examples, the silica matrix does not comprise 40% or more, 50% or more, 60% or more, or 70% or more Q4 silicon atoms. An inorganic material matrix (e.g., silica matrix) may comprise a plurality of first domains, where adjacent first domains are linked by a thinner (e.g., linking) second domain, may be referred to as “pearl chain” structure.

Without intending to be bound by any particular theory, it is considered that a silica matrix/silica matrices comprising/independently comprise a plurality of first domains, where adjacent first domains are linked by a thinner (e.g., linking) second domain are able to deform (e.g., exhibit a bending modulus that allows the silica nanocage to adopt a shape with at least one dimension that is smaller than the diameter of the silica nanocage that is not deformed) and pass thru an aperture having an opening smaller than the longest dimension of this silica nanocage. In various examples, a silica nanocage having a longest dimension greater than 6 nm (generally considered to be the limit of renal clearance of an individual, such as, for example, a human, a non-human animal, or the like) can clear (e.g., pass thru) the kidneys of an individual, such as, for example, a human, a non-human animal, or the like).

The inorganic nanocages can have desirable surface area. The inorganic nanocages may have a surface area of 500 to 800 m²/g. The surface area may be determined by methods known in the art. In an example, the surface area is determined by BET analysis of nitrogen sorption isotherms.

The inorganic nanocages may be functionalized (e.g., as described herein). The inorganic nanocages can be functionalized using various methods (e.g., as described herein). At least a portion of a surface (e.g., at least a portion of an exterior surface and/or at least a portion of an interior surface of the inorganic nanocages may be functionalized (e.g., covalently functionalized and/or non-covalently functionalized).

The inorganic nanocages may be selectively functionalized. The functionalization may be the same for the interior surface and exterior surface of the inorganic nanocages or may be different for the interior surface and exterior surface of the inorganic nanocages.

The interior (inner) and exterior (outer) surface of the inorganic nanocages may be selectively modified with desired functional groups via both covalent and non-covalent interactions for different applications. For example, the exterior surface of the inorganic nanocages can be covalently functionalized with polyethylene glycol for improving bio-compatibility. In another example, the outer surface of the inorganic nanocages can be further covalently functionalized with ligand groups for theranostics applications, including but not limited to peptides, RNAs, DNAs, drug molecules, sensor ligands, antibodies, antibody fragments, radioisotopes, and the like, and combinations thereof. The silica matrix of the silica nanocages may be covalently labeled with a fluorescent dye to endow the cages with fluorescence properties.

In an aspect, the present disclosure provides compositions comprising inorganic nanocages of the present disclosure. The compositions can comprise one or more type(s) (e.g., having different average size and/or one or more different compositional feature(s)).

The compositions may include one or more standard pharmaceutically acceptable carrier(s). Non-limiting examples of compositions include solutions, suspensions, emulsions, solid injectable compositions that are dissolved or suspended in a solvent before use, and the like. The injections may be prepared by dissolving, suspending or emulsifying one or more of the active ingredient(s) in a diluent. Examples of diluents are distilled water for injection, physiological saline, vegetable oil, alcohol, and a combination thereof. Further, the injections may contain stabilizers, solubilizers, suspending agents, emulsifiers, soothing agents, buffers, preservatives, and the like. The injections, are sterilized in the final formulation step or prepared by sterile procedure. The pharmaceutical composition of the disclosure may also be formulated into a sterile solid preparation, for example, by freeze-drying, and can be used after sterilized or dissolved in sterile injectable water or other sterile diluent(s) immediately before use. Non-limiting examples of pharmaceutically acceptable carriers can be found in: Remington: The Science and Practice of Pharmacy (2005) 21st Edition, Philadelphia, Pa. Lippincott Williams & Wilkins.

A composition may comprise a plurality inorganic nanocages (e.g., silica inorganic nanocages). Any of the inorganic nanocages may be surface functionalized with one or more type of polyethylene glycol group(s) (e.g., polyethylene glycol groups, functionalized (e.g., functionalized with one or more ligand(s) and/or a reactive group) polyethylene glycol groups, or a combination thereof). Any of the inorganic nanocages may have a dye or combination of dyes (e.g., a NIR dye) encapsulated therein. The dye molecules may be covalently bound to the inorganic nanocages. The inorganic nanocages may be made by a method of the present disclosure.

The composition can comprise additional components. For example, the composition can also comprise a buffer suitable for administration to an individual (e.g., a mammal such as, for example, a human). The buffer may be a pharmaceutically-acceptable carrier.

In an aspect, the present disclosure provides uses of inorganic nanocages. In various examples, inorganic nanocages or a composition comprising inorganic nanocages are used in delivery and/or imaging methods.

The ligands carried by the inorganic nanocages may include diagnostic and/or therapeutic agents (e.g., drugs). Examples of therapeutic agents include, but are not limited to, chemotherapeutic agents, antibiotics, antifungal agents, antiparasitic agents, antiviral agents, and combinations thereof. An affinity ligand may be also be conjugated to the inorganic nanocages to allow targeted delivery of the inorganic nanocages. For example, the inorganic nanocages may be conjugated to a ligand which is capable of binding to a cellular component (e.g., on the cell membrane or in the intracellular compartment) associated with a specific cell type. The targeted molecule can be a tumor marker or a molecule in a signaling pathway. The ligand can have specific binding affinity to certain cell types, such as, for example, tumor cells. In certain examples, the ligand may be used for guiding the inorganic nanocages to specific areas, such as, for example, liver, spleen, brain or the like. Imaging can be used to determine the location of the inorganic nanocages in an individual.

The inorganic nanocages or compositions comprising inorganic nanocages may be administered to individuals for example, in pharmaceutically-acceptable carriers, which facilitate transporting the inorganic nanocages from one organ or portion of the body to another organ or portion of the body. Examples of individuals include animals such as human and non-human animals. Examples of individuals also include mammals.

Compositions comprising the present inorganic nanocages can be administered to an individual by any suitable route—either alone or as in combination with other agents. Administration can be accomplished by any means, such as, for example, by parenteral, mucosal, pulmonary, topical, catheter-based, or oral means of delivery. Parenteral delivery can include, for example, subcutaneous, intravenous, intramuscular, intra-arterial, and injection into the tissue of an organ. Mucosal delivery can include, for example, intranasal delivery. Pulmonary delivery can include inhalation of the agent. Catheter-based delivery can include delivery by iontophoretic catheter-based delivery. Oral delivery can include delivery of an enteric coated pill, or administration of a liquid by mouth. Transdermal delivery can include delivery via the use of dermal patches.

Following administration of a composition comprising the present inorganic nanocages, the path, location, and clearance of the inorganic nanocages can be monitored using one or more imaging technique(s). Examples of suitable imaging techniques include fluorescence imaging (e.g. using the Artemis Fluorescence Camera System) or positron emission tomography when using a radiolabel attached to the nanocages.

The inorganic nanocages (e.g., silica nanocages) may exhibit desirable renal clearance. In various examples, the inorganic nanocages (e.g., silica nanocages) to do not exhibit substantial uptake in one or more of an individual's organ(s) of the reticuloendothelial system (RES), such as, for example, liver, spleen, or the like or a combination thereof. By substantial uptake it is meant that less than 10% of the inorganic nanocages (e.g., silica nanocages), less than 5% of the inorganic nanocages (e.g., silica nanocages), less than 1% of the inorganic nanocages (e.g., silica nanocages), less than 0.1% of the inorganic nanocages (e.g., silica nanocages) are observed in an individual's organ(s), such as for example, liver, spleen, or the like, or a combination thereof 3 or more, 5 or more, or 7 more days after administration of the inorganic nanocages (e.g., silica nanocages). The presence and/or absence of inorganic nanocages (e.g., silica nanocages) in an individual's organ(s) can be determined by imaging methods. In various examples, the presence and/or absence of inorganic nanocages (e.g., silica nanocages) in an individual's organ(s) is/are determined by positron emission tomography (PET), optical imaging methods, or the like, or a combination thereof, examples of which are provided herein. Without intending to be bound by any particular theory, it is considered that the uptake of inorganic nanocages (e.g., silica nanocages) is correlated with the diffusion coefficient of the inorganic nanocages (e.g., silica nanocages).

This disclosure provides a method for imaging biological material such as cells, extracellular components, or tissues comprising contacting the biological material with inorganic nanocages comprising one or more dye(s), or compositions comprising the inorganic nanocages; directing excitation electromagnetic (e/m) radiation, such as light, on to the tissues or cells thereby exciting the dye molecules; detecting e/m radiation emitted by the excited dye molecules; and capturing and processing the detected e/m radiation to provide one or more image(s) of the biological material. One or more of these step(s) can be carried out in vitro or in vivo. For example, the cells or tissues can be present in an individual or can be present in culture. Exposure of cells or tissues to e/m radiation can be effected in vitro (e.g., under culture conditions) or can be effected in vivo. For directing e/m radiation at cells, extracellular materials, tissues, organs and the like within an individual or any portion of an individual's body that are not easily accessible, fiber optical instruments can be used.

For example, a method for imaging of a region within an individual comprises (a) administering to the individual inorganic nanocages or a composition of the present disclosure comprising one or more dye molecule(s); (b) directing excitation light into the individual, thereby exciting at least one of the one or more dye molecule(s); (c) detecting excited light, the detected light having been emitted by said dye molecules in the individuals as a result of excitation by the excitation light; and (d) processing signals corresponding to the detected light to provide one or more image(s) (e.g. a real-time video stream) of the region within the individual.

Since the fluorescent inorganic nanocages are brighter than free dye, fluorescent inorganic nanocages can be used for tissue imaging, as well as to image the metastasis tumor. Additionally or alternatively, radioisotopes can be further attached to the ligand groups (e.g., tyrosine residue or chelator) of the ligand-functionalized inorganic nanocages or to the silica matrix of the PEGylated particles without specific ligand functionalization for photoinduced electron transfer imaging. If the radioisotopes are chosen to be therapeutic, such as, for example, ²²⁵Ac or ¹⁷⁷Lu, this in turn would result in inorganic nanocages with additional radiotherapeutic properties.

For example, drug-linker conjugate, where the linker group can be specifically cleaved by enzyme or acid condition in tumor for drug release, can be covalently attached to the functional ligands on the particles for drug delivery. For example, drug-linker-thiol conjugates can be attached to maleimido-PEG-particles through thiol-maleimido conjugation reaction post the synthesis of maleimido-PEG-particles. Additionally, both drug-linker conjugate and cancer targeting peptides can be attached to the particle surface for drug delivery specifically to tumor.

The present disclosure provides methods of using one or more inorganic nanocage(s) and/or one or more composition(s) comprising one or more inorganic nanocage(s) comprising administering the inorganic nanocage(s) and/or one or more composition(s) to treat cancer. At least a portion of or all of the inorganic nanocages may be silica nanocages. The inorganic nanocages (e.g., silica nanocages) may exhibit desirable renal clearance.

In various examples, a method of treating cancer in an individual comprises administering to the individual a therapeutically effective amount of a composition comprising one or more inorganic nanocage(s) (e.g., silica nanocage(s)) of the present disclosure, where the individual's cancer is treated. At least a portion of or all of the inorganic nanocages may be silica nanocages. The inorganic nanocages (e.g., silica nanocages) may exhibit desirable renal clearance. At least a portion of the inorganic nanocage(s) (e.g., silica nanocages) may comprise a drug and at least a portion of the drug is released from the inorganic nanocage(s) (e.g., silica nanocages) and/or at least a portion of the inorganic nanocage(s) (e.g., silica nanocages) may comprise a radioisotope (which may result in radiotherapy). At least a portion of the inorganic nanocage(s) (e.g., silica nanocages) may comprise one or more display group(s) that target(s) the cancer. A method may further comprise visualization of at least a portion of the cancer using optical imaging (e.g., fluorescence imaging), PET imaging, CT imaging, or a combination thereof. A method may further comprise treatment of the individual with one or more known cancer therapy/therapies in conjunction with administration of the inorganic nanocage(s) (e.g., silica nanocages) (e.g., before and/or after and/or at the same time as the administration of the inorganic nanocage(s) (e.g., silica nanocages)).

A method may be carried out in combination with one or more known therapy/therapies. Non-limiting examples of known therapies include other agents used to treat cancer (such as, for example, drugs, which may be chemotherapeutic drugs), immunotherapy, radiation, surgery, and the like. A method may be carried out in conjunction with an imaging method. In various examples, a method of treating cancer is carried out in conjunction with an imaging method of the present disclosure.

Various cancers may be treated via a method of the present disclosure. Non-limiting examples of cancers include leukemia, lung cancer (e.g., non-small cell lung cancer), dermatological cancers, premalignant lesions of the upper digestive tract, malignancies of the prostate, malignancies of the brain, malignancies of the breast, colon cancer, solid tumors, melanomas, and the like, and combinations thereof. In various examples, one or more inorganic nanocage(s) (e.g., silica nanocage(s)) and/or one or more composition(s) comprising one or more inorganic nanocage(s) (e.g., silica nanocage(s))described herein is administered to an individual in need of treatment using any known method and route, including, but not limited to, parenteral, mucosal, topical, catheter-based, oral, intravenous, or transdermal means of delivery, or the like. Parenteral delivery can include, for example, subcutaneous, intravenous, intramuscular, intercranial, intra-arterial delivery, which may be injection into the tissue of an organ.

Compositions comprising one or more inorganic nanocage(s) can be administered to an individual by any suitable route—either alone or in combination with other agents. Administration can be accomplished by any means as described herein.

A method can be carried out in an individual in need of treatment who has been diagnosed with or is suspected of having cancer. A method can also be carried out in an individual who have a relapse or a high risk of relapse after being treated for cancer.

An individual in need of treatment may be a human or non-human mammal or other animal. Non-limiting examples of non-human mammals include cows, horses, pigs, mice, rats, rabbits, cats, dogs, or other agricultural mammals, pets, or service animals, and the like.

In various examples, silica nanocages are used in a therapeutically effective amount (e.g., administered to an individual in need of treatment). The term “therapeutically effective amount” as used herein refers to an amount of an agent sufficient to achieve, in a single or multiple doses, the intended purpose of treatment. Treatment does not have to lead to complete cure, although it may. Treatment may mean alleviation of one or more of the symptom(s) (e.g., may at least shrink a solid tumor) and/or marker(s) of the indication. The exact amount desired or required will likely vary depending on the particular silica nanocage(s) or composition(s) used, its mode of administration, patient specifics, and the like. An appropriate effective amount may be determined by one of ordinary skill in the art informed by the instant disclosure using only routine experimentation. Treatment can be effected over a short period, over a medium term, or can be a long-term treatment, such as, for example, within the context of a maintenance therapy. Treatment can be continuous or intermittent.

The steps of the methods described in the various embodiments and examples disclosed herein are sufficient to carry out the methods of the present disclosure. Thus, in an embodiment, a method consists essentially of a combination of the steps of the methods disclosed herein. In another embodiment, the method consists of such steps.

The following Statements describe various examples of inorganic nanocages, methods of making inorganic nanocages, and uses of inorganic nanocages of the present disclosure:

Statement 1. A method of making inorganic nanocages (e.g., non-metal oxide nanocages, transition metal nanocages, and transition metal oxide nanocages) comprising

forming a reaction mixture comprising
one or more precursor(s);
one or more surfactant(s) (e.g., surfactant(s) including positively charged groups or a surfactant including negatively charged groups);
one or more pore expander(s) (e.g., hydrophobic pore expander(s)); and

holding the reaction mixture at a time (t¹) and temperature (T¹), whereby inorganic nanocages (e.g. inorganic nanocages having an average size of a longest dimension (e.g., diameter) less than 30 nm) are formed; and

optionally, adding a terminating agent (which may be a capping agent) and/or a reductant (which may be a capping agent) to the reaction mixture.

The structural features (e.g., arms, vertices, and the like) of the inorganic nanocages (e.g., silica nanocages), may have modulated thickness (e.g., one or more modulated dimension(s) normal to a long axis of the inorganic material matrix (e.g., silica matrix)). In various examples, the inorganic material matrix (e.g., silica matrix) has a modulated diameter, modulated radius, or the like. In various examples, the inorganic material (e.g., silica matrix) of a structural feature has a plurality of domains (e.g., silica domains), where at least two domains (which may referred to as first domains) are connected (e.g., covalently bonded) by, for example, a plurality of Si—O—Si bonds), an inorganic material domain (e.g., silica domain) (which may be referred to as a second domain (e.g., second silica domain)) and this domain (e.g., second silica domain) has a dimension normal to a long axis of the inorganic material matrix (e.g., silica matrix) that is 50% or less (e.g., 10-50%, including all 0.1% values and ranges therebetween) than a dimension normal to a long axis of the inorganic material matrix (e.g., silica matrix) of one or both of the two domains (e.g., first domain(s)).
Statement 2. A method according to Statement 1, where

the one or more surfactant(s) is/are chosen from C₁₀ to C₁₆ alkyltrimethylammonium halides (e.g., cetyltrimethylammonium bromide (CTAB), decyltrimethylammonium bromide (C₁₀TAB), dodecyltrimethylammonium bromide (C₁₂TAB), myristyltrimethylammonium bromide (C₁₄TAB), octadecyltrimethylammonium bromide (C₁₈TAB), and the like), sodium dodecyl sulfate (SDS), N-myristoyl-L-glutamic acid (C14GluA), and combinations thereof, and/or

the one or more pore expander(s) is/are chosen from trialkylated benzene (e.g., 1,2,4-trimethylbenzene (TMB), and the like), polymer monomers (e.g., stryrenes, alkylstyrenes (e.g., methyl styrene, and the like), hydrophobic solvents (e.g., alkanes (e.g., hexane and the like), cycloalkanes (e.g., cyclohexane and the like), benzene, alkylated benzene (e.g., toluene and the like), chlorinated alkanes (e.g., chloroform and the like)), and the like, and combinations thereof.

Statement 3. A method according to Statements 1 or 2, where the one or more surfactant(s) is/are present in the reaction mixture at a concentration ranging from 1 mg/mL to 50 mg/mL and/or the one or more pore expander(s) is/are present at a concentration ranging from 3 mg/mL to 100 mg/mL.
Statement 4. A method according to any on one the preceding Statements, where the molar ratio of the one or more surfactant(s) to the one or more pore expander(s) is 1:100 to 10:1.
Statement 5. A method according to any one of the preceding Statements, where the one or more precursor(s) is/are one or more non-metal oxide precursor(s) (e.g., non-metal oxide precursor(s) chosen from silica precursors (e.g., tetraalkoxysilanes (e.g., tetramethylorthodsilicate (TMOS), tetraethylorthosilicate (TEOS), tetrapropylorthosilicate (TPOS), and the like), alkyltrialkoxysilanes (e.g., methyltrimethylorthosilicate), functionalized non-metal oxide precursors (e.g., (3-aminopropyl)triethoxysilane (APTES), (3-aminopropyl)trimethoxysilane (APTMS), (3-mercaptopropyl)trimethoxysilane (MPTMS), and the like and combinations thereof), and the like, and combinations thereof).
Statement 6. A method according to Statement 5, where at least one of non-metal oxide precursors comprises one or more functional group(s) (e.g., fluorescent dye(s) (e.g., dye-silane conjugate(s), such as, for example, ATTO647N-silane) and/or theranostic functional moiet(ies) (e.g., drugs and a fluorescent dyes (e.g., drug-dye-silane conjugate(s), such as, for example, DFO-ATTO647N-silane)), peptide(s) and fluorescent dye(s) (e.g., peptide-dye-silane conjugate(s), such as, for example, cRGDY-ATTO647N-silane)).
Statement 7. A method according to Statements 5 or 6, where the terminating agent is a PEG-silane.
Statement 8. A method according to Statement 7, where before or after the PEG-silane conjugate is added, adding a PEG-silane conjugate comprising a ligand is added at room temperature to the reaction mixture,
holding the resulting reaction mixture at a time (t²) and temperature (T²), and subsequently heating the resulting reaction mixture at a time (t³) and temperature (T³), whereby inorganic nanocages surface functionalized with PEG groups comprising a ligand are formed.
Statement 9. A method according to Statements 7 or 8, where at least a portion of or all of the PEG-silane has a reactive group on a terminus of the PEG group opposite the terminus conjugated to the silane group of the PEG-silane conjugate and after formation of the inorganic nanocages surface functionalized with PEG groups having a reactive group, and, optionally, PEG groups, are reacted with a second ligand functionalized with a second reactive group thereby forming inorganic nanocages surface functionalized with polyethylene groups functionalized with a second ligand and, optionally, PEG groups.
Statement 10. A method according to any one of Statements 7-9, where at least a portion of or all of the PEG-silane has a reactive group on a terminus of the PEG moiety opposite the terminus conjugated to the silane moiety of the PEG-silane conjugate and after formation of the inorganic nanocages surface functionalized with PEG groups and, optionally having a reactive group, and, optionally, PEG groups, are reacted with a second ligand functionalized with a second reactive group thereby forming inorganic nanocages surface functionalized with polyethylene groups functionalized with a second ligand and, optionally, PEG groups,
Statement 11. A method according to any one of Statements 5-10, where the reaction mixture further comprises one or more solvent(s) (e.g., where the solvent is water and the pH of the reaction mixture is 6 or greater (e.g., 6-9)).
Statement 12. A method according to any one of Statements 1-4, where the one or more precursor(s) is/are chosen from transition metal salts, transition metal alkoxides, transition metal coordination complexes, organometallic compounds, and combinations thereof.
Statement 13. A method according to Statement 12, where the transition metal salts are gold salts (e.g., gold chlorides, gold chloride hydrates, and the like, combinations thereof), silver salts (e.g., silver halides, silver nitrates, and the like, and combinations thereof), palladium salts (e.g. sodium tetrachloropalladate(II)), platinum salts (e.g. potassium tetrachloroplatinate(II)), zirconium salts (e.g., zirconium(IV) sulfate and hydrates thereof), iron salts (e.g. iron(II) perchlorate hydrate, iron(III) nitrate nonahydrate), rhodium salts, copper salts, nickel salts, tantalum salts, hafnium salts, niobium salts, and combinations thereof.
Statement 14. A method according to Statements 12 or 13, where the terminating agent is a reducing terminating agent.
Statement 15. A method according to Statement 14, where the reducing terminating agent is chosen from tetrakis(hydroxymethyl)phosphonium chloride (THPC), bis[tetrakis(hydroxymethyl)phosphonium] sulfate (THPS), and the like, and combinations thereof.
Statement 16. A method according to any one of Statements 1-4, where the one or more precursor(s) is/are one or more transition-metal oxide precursor(s) chosen from transition metal alkoxides, transition metal salts, and combinations thereof.
Statement 17. A method according to Statement 16, where the transition metal alkoxides are vanadium alkoxides (e.g., vanadium oxytriisopropoxide), titanium alkoxides (e.g., titanium isopropoxide), niobium alkoxides (e.g., niobium(V) ethoxide), zirconium alkoxides (e.g., zirconium(IV) sulfate and hydrates thereof), tantalum alkoxides (e.g. tantalum(V) ethoxide), hafnium alkoxides (e.g., hafnium(IV) tert-butoxide), copper alkoxides, nickel alkoxides, iron alkoxides, and combinations thereof.
Statement 18. A method according to any one of Statements 1-4, where at least a portion of a surface (e.g., at least a portion of an exterior surface and/or at least a portion of an interior surface of the inorganic nanocages) is functionalized (e.g., covalently functionalized and/or non-covalently functionalized)).
Statement 19. A method according to any one of the preceding Statements, where the method further comprises isolation/separation (e.g., using size exclusion chromatography, high performance liquid chromatography, and gel permeation chromatography) of at least a portion of the inorganic nanocages from the reaction mixture.
Statement 20. An inorganic nanocage, which may be symmetrical (e.g., highly symmetrical), inorganic nanocage having a longest dimension (e.g., diameter) less than 30 nm (e.g., less than 5 nm to about 20 nm, including all 0.1 nm ranges and values therebetween, or about 5 nm to about 20 nm, including all 0.1 nm ranges and values therebetween) comprising an inorganic material, the inorganic nanocage comprising:

an interior (e.g., an interior space of the inorganic nanocage) and an exterior (e.g., an exterior space of the inorganic nanocage), where the interior and the exterior each have a surface;

vertices (e.g., having a longest dimension (e.g., diameter) of about 1 nm to about 5 nm, including all 0.1 nm values and ranges therebetween);

arms connecting adjacent/nearby vertices (e.g., having a longest dimension (e.g., diameter) of about less than 1 nm to about 3 nm, including all 0.1 nm values and ranges therebetween); and

apertures (which may be referred to as windows or open windows (e.g., pores)), which can connect the exterior space to the interior space (e.g., having a longest dimension (e.g., a diameter) of about 1 nm to about 10 nm, including all 0.1 nm values and ranges therebetween (e.g., about 1 nm to about 5 nm)).

Statement 21. An inorganic nanocage according to Statement 20, where the interior surface and/or exterior of the inorganic nanocage (e.g., at least a portion of an interior surface and/or at least a portion of exterior surface) is functionalized/modified with at least one functional group (e.g., at least one functional group that can have a covalent and/or non-covalent interaction (e.g., covalent functionalization (e.g., attachment) and/or non-covalent functionalization (e.g., attachment)) with another functional group), where when there is more than one functional group, the functional groups are the same or different, or some are the same and some are different.
Statement 22. An inorganic nanocage according to Statement 21, where the at least one functional group is chosen from peptide groups (e.g., cancer targeting peptide groups), nucleic acid groups (e.g., RNA groups, DNA groups, and the like, and combinations thereof), drug groups, sensor ligands, antibody groups, antibody fragment groups, groups comprising a radioisotope, and the like, and combinations thereof.
Statement 23. An inorganic nanocage according to any one of Statements 20-22, where the inorganic material is chosen from non-metal oxides (e.g., non-metal oxide groups such as, for example, —O—Si—O—, and the like), transition metal oxides (e.g., transition-metal oxide groups such as, for example, —O—V-O—), metals, and combinations thereof.
Statement 24. An inorganic nanocage according to Statement 23, where the non-metal oxide is (e.g., the non-metal oxide groups are) chosen from silicon oxide, aluminosilicate, and the like, and combinations thereof and, optionally, the inorganic nanocage further comprises aluminum oxide groups.
The structural features (e.g., arms, vertices, and the like) of the inorganic nanocage may have modulated thickness (e.g., one or more modulated dimension(s) normal to a long axis of the silica matrix). For example, in the case where the non-metal oxide is a non-metal oxide (e.g., silicon oxide or the like), the non-metal oxide matrix (e.g., silica matrix) has a modulated diameter, modulated radius, or the like. In various examples, the non-metal matrix (e.g., silica matrix) of a structural feature has a plurality of non-metal oxide domain (e.g., silica domains), where two domains (which may referred to as first domains) are connected (e.g., covalently bonded by), for example, a plurality of Si—O—Si bonds) by a non-metal oxide (e.g., silica domain) (which may be referred to as a second non-metal oxide (e.g., second silica domain) and this domain (e.g., second non-metal oxide domain, such as, for example, silica domain) has a dimension normal to a long axis of the silica matrix that is 50% or less (e.g., 10-50%, including all 0.1% values and ranges therebetween) than a dimension normal to a long axis of the non-metal oxide matrix (e.g., silica matrix) of one or both of the two domains (e.g., first domain(s)).
Statement 25. An inorganic nanocage according to Statement 23, where the transition metal oxide is chosen from vanadium oxide, titanium oxide, niobium oxide, iron oxide, copper oxide, nickel oxide, hafnium oxide, zirconium oxide, tantalum oxide, and the like, and combinations thereof.
Statement 26. An inorganic nanocage according to Statement 23, where the transition metal is chosen from silver, gold, palladium, platinum, rhodium, and the like, and combinations thereof.
Statement 27. An inorganic nanocage according to any one of Statements 20-26, where the inorganic nanocage is dodecahedral (5¹²), icosahedral, cubic, hexahedral, tetrahedral, octahedral, tetrakaidecahedral, pentakaidecahedral, hexakaidecahedron, rhombic dodecahedral, trapezo-rhombic, buckyball-like (5¹²6²⁰), 3³4³, 4⁴5⁴, 4³5⁶6³, 3³4³5⁹, 5¹²6², 4⁶6⁸, 5¹²6³, 5¹²6⁴, 4³5⁹6²7³, or 5¹²6⁸.
Statement 28. An inorganic nanocage according to any one of Statements 20-27, where the inorganic nanocage has a specific surface area 500 to 800 square meter per gram.
Statement 29. An inorganic nanocage according to any one of Statements 20-28, where the highly symmetrical nanocage is used as a catalyst, drug delivery agent, diagnostic agent, as a therapeutic agent, a theranostic agent (e.g., acts as both a diagnostic agent and a therapeutic agent) or the like, or a combination thereof.
Statement 30. An inorganic nanocage according to any one of Statements 20-29, where the inorganic nanocage has a longest dimension (e.g., a longest linear dimension, such as, for example, a diameter) of 5 to 15 nm, including every 0.1 nm value and range therebetween (e.g., 5-10 nm).
Statement 31. A composition comprising one or more inorganic nanocage(s) of the present disclosure (e.g., one or more inorganic nanocage(s) of any one of Statements 20-30 and/or one or more inorganic nanocage(s) made by a method of any one of Statements 1-19).
Statement 32. A method for imaging of a region within an individual comprising:

administering to the individual one or more inorganic nanocage(s) and/or a composition comprising one or more inorganic nanocage(s) of the present disclosure (e.g., inorganic nanocages of any one of Statements 20-30 and/or inorganic nanocage(s) made by a method of any one of Statements 1-19 or a composition of Statement 31), where the inorganic nanocage(s) comprise one or more dye molecule(s), one or more radioisotope(s), one or more iodides, or the like; or a combination thereof;

directing excitation electromagnetic radiation into the individual, thereby exciting at least one of the one or more dye molecule(s);

detecting excited electromagnetic radiation, the detected electromagnetic radiation having been emitted by said dye molecules in the individuals as a result of excitation by the excitation electromagnetic radiation; and

processing signals corresponding to the detected electromagnetic radiation to provide one or more image(s) of the region within the individual.

The silica nanocages may exhibit desirable renal clearance.

Statement 33. A method according to Statement 32, where the imaging is optical (e.g., fluorescence imaging), PET imaging, CT imaging, or a combination thereof.
Statement 34. A method of treating cancer in an individual comprising administering to the individual a therapeutically effective amount of one or more inorganic nanocage(s) and/or a composition comprising one or more inorganic nanocage(s) of the present disclosure (e.g., inorganic nanocages of any one of Statements 20-30 and/or inorganic nanocage(s) made by a method of any one of Statements 1-19 or a composition of Statement 31), wherein the individual's cancer is treated. At least a portion of or all of the inorganic nanocages may be silica nanocages. The silica nanocages may exhibit desirable renal clearance.
Statement 35. The method of Statement 34, wherein at least a portion of the inorganic nanocage(s) (e.g., silica nanocages) comprises a drug and at least a portion of the drug is released from the inorganic nanocage(s) (e.g., silica nanocages).
Statement 36. The method of Statement 34 or 35, wherein at least a portion of the inorganic nanocage(s) (e.g., silica nanocages) comprises one or more display group(s) that target(s) the cancer.
Statement 37. The method of any one of Statements 34-36, further comprising visualization of at least a portion of the cancer using optical imaging (e.g., fluorescence imaging), PET imaging, CT imaging, or a combination thereof.
Statement 38. The method of any one of Statements 34-37, further comprising treatment of the individual with one or more known cancer therapy/therapies) in conjunction with administration of the inorganic nanocage(s) (e.g., silica nanocages) (e.g., before and/or after and/or at the same time as the administration of the inorganic nanocage(s) (e.g., silica nanocages)).
Statement 39. The method of any one of Statements 34-38, wherein the cancer is chosen from brain cancers, melanomas, prostate cancer, breast cancer, lung cancer, and the like, and combinations thereof. The cancer may be a solid tumor.
Statement 40. The method of any one of Statements 34-39, wherein the individual is a human individual or a non-human individual.

The following examples are presented to illustrate the present disclosure. They are not intended to be limiting in any matter.

### Example 1

This example provides a description of nanocages and synthesis of nanocages of the present disclosure.

Employing a combination of cryo-EM and single-particle 3D reconstruction, ultrasmall silica cages (“silicages”) with dodecahedral structure were prepared. This highly symmetric self-assembled cage forms via arrangement of primary silica clusters in aqueous solutions on the surface of oppositely charged surfactant micelles. These nanoscale cages may be used as building blocks for a wide range of advanced functional materials applications.

Chemicals and materials. All chemicals were used as received. Cetyltrimethylammonium bromide (CTAB), ammonia (2 M in ethanol), mesitylene (1,3,5 trimethylbenzene, TMB), tetramethyl orthosilicate (TMOS), gold chloride trihydrate (HAuCl₄.3H₂O), silver nitrate (AgNO₃), tetrakis(hydroxymethyl)phosphonium chloride (THPC), dimethyl sulfoxide (DMSO), acetic acid, and ethanol were purchased from Sigma-Aldrich. Vanadium oxytriisopropoxide was purchased from Alfa Aesar. Anhydrous potassium carbonate (K₂CO₃) was purchased from Mallinckrodt. Anhydrous ethanol was purchased from Koptec. Silane modified monofunctional polyethylene glycol (PEG-silane) with molar mass around 500 g/mol (6-9 ethylene glycol units) was purchased from Gelest. Carbon film coated copper grids for TEM and C-Flat holey carbon grids for cryo-EM were purchased from Electron Microscopy Sciences.

Synthesis, TEM, and cryo-EM characterization of silicages. Silicages were synthesized in aqueous solution through surfactant directed silica condensation. 125 mg of CTAB was first dissolved in 10 ml of ammonium hydroxide solution (0.002 M). 100 μL of TMB was then added to expand CTAB micelle size. The solution was stirred at 600 rpm at 30° C. overnight, followed by the addition of 100 μL of TMOS. The reaction was then left at 30° C. overnight under stirring at 600 rpm.

To prepare cryo-EM samples, 5 μL of the native reaction solution was applied to glow discharged CF-4/2-2C Protochips C-Flat holey carbon grids, blotted using filter paper and plunged into a liquid mixture of 37% ethane and 63% propane at −194° C. using an EMS plunge freezer. Cryo-EM images were acquired on a FEI Tecnai F20-ST TEM operated at an acceleration voltage of 200 kV using a Gatan Onus CCD camera. All cryo-EM images used for reconstruction were acquired at the same magnification, with a pixel size of 0.16 nm, and nominal defocus was kept between 1 μm and 2 μm.

To prepare dry-state TEM samples, 100 μL of PEG-silane was added into the reaction solution. The reaction solution was left at 30° C. overnight under stirring at 600 rpm to surface modify silicages covalently with PEGs to improve their dispersity on TEM grids. Afterwards, 30 μL of the reaction solution was dropped onto a cupper grid coated with a continuous carbon film, and blotted using filter paper. TEM images were acquired using a FEI Tecnai T12 Spirit microscope operated at an acceleration voltage of 120 kV. In order to improve the signal to noise ratio in recorded images, TEM sample grids were plasma etched for 5 seconds before TEM characterization, and a series of images was acquired of the same sample area, which was then averaged.

In order to quench individual primary silica clusters formed at the very early stages of cage formation, 100 μL of PEG-silane was added into the reaction solution about three minutes after the addition of TMOS. The rest of the procedures, including particle synthesis, dry-state TEM sample preparation, and TEM characterization, were the same as described above.

Synthesis and TEM characterization of metal cage-like structures. The gold and silver cage-like structures were prepared by the reduction of metal precursors, HAuCl₄.3H₂O and AgNO₃, respectively, in the presence of micelles with the same water:CTAB:TMB ratio as for the silicage work. In a typical batch, 50 mg of CTAB was dissolved in 4 mL of water at 30° C., then 40 μL of TMB and 200 μL of ethanol were added to the mixture. After stirring the reaction at 30° C. overnight at 600 rpm, 16 μL of either HAuCl₄.3H₂O (25 mM) or AgNO₃ (25 mM) was added, followed after 5 minutes by 8 μL of THPC (68 mM). After another 5 minutes, 6 μL of potassium carbonate (0.2 M) was finally added.

Dry-state TEM samples for gold and silver cage-like structures were prepared after one day and 6 hours of reaction, respectively, due to different reaction rates. In both cases, the samples were prepared by drying 8 μL of the native reaction mixture diluted three times in ethanol on a TEM grid in air overnight. In order to remove the thick CTAB layer before imaging, the grid was immersed in ethanol for 2 minutes and then dried in air. TEM images of metal cage-like structures were acquired using a FEI Tecnai T12 Spirit microscope operated at an acceleration voltage of 120 kV.

Synthesis and TEM characterization of vanadium oxide cage-like structures. The vanadium oxide cage-like structures were prepared based on sol-gel chemistry very similar to the silicages, using vanadium oxytriisopropoxide as the precursor. In a typical batch, 50 mg of CTAB was dissolved in 4 ml of water at 30° C., then 40 μL of TMB was added to the mixture. After stirring the reaction at 30° C. overnight at 600 rpm, 50 μL of vanadium oxytriisopropoxide diluted in 100 μL of DMSO was added to the reaction.

Dry-state TEM samples for vanadium oxide cage-like structures were prepared after one day of reaction by drying on a TEM grid 8 μL of the native reaction mixture diluted 10 times in water. At such dilution, the amount of CTAB was low enough so that the TEM samples did not require any plasma cleaning or soaking in ethanol prior to imaging. The TEM images of vanadium oxide cages were acquired using a FEI Tecnai T12 Spirit microscope operated at an acceleration voltage of 120 kV.

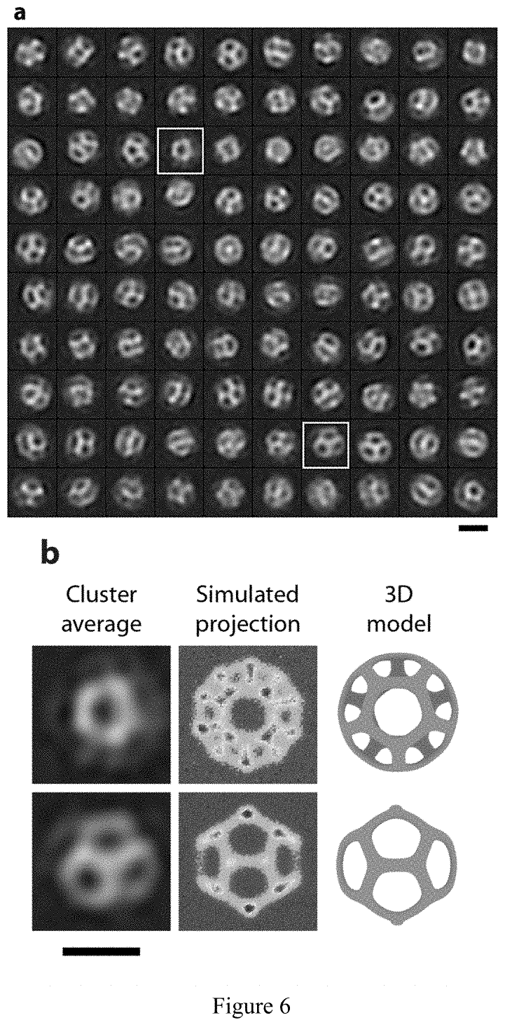

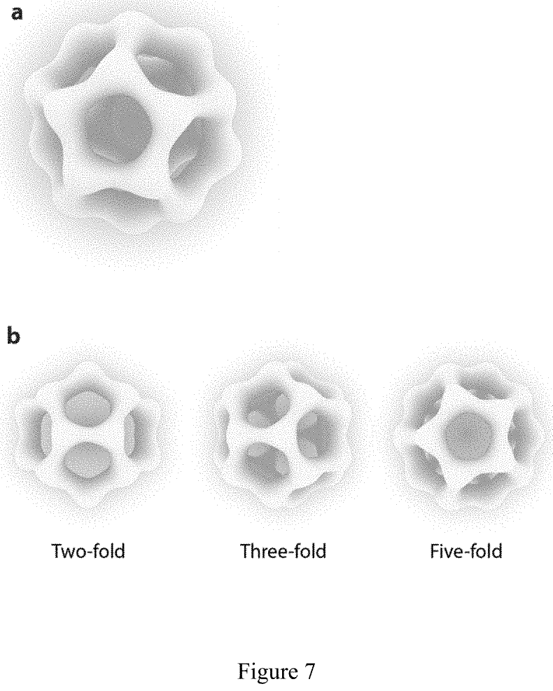

Particle reconstruction. The “Hetero” model-based maximum likelihood algorithm was used which can simultaneously estimate: (1) a reconstruction for each type of particle shown in the images, (2) the type of particle shown in each image, and (3) the projection orientation for each image. Such joint estimation is a central feature of the algorithm and is a natural approach to process data from complicated mixtures. The estimates in (2) and (3), which are based on 3D structure, are independent of the clustering of 2D images, which is based on pixel values (e.g., FIG. 6). In addition to the Hetero algorithm, the widely used RELION 2.1 system was applied to compute equivalent two-class and single-class reconstructions. The images were corrected for the CTF by phase flipping.

Particle purification. To remove CTAB and TMB from the cages, after adding PEG-silane and stirring at room temperature for a day, the solution was heat-treated at 80° C. overnight to further enhance the covalent attachment of PEG-silane to the silica surface of the silicages. The PEGylated nanocages were first dialyzed (molecular weight cut off, MWCO, 10 kDa) in a mixture of acetic acid, ethanol, and water (volume ratio 7:500:500) for three days, and were then dialyzed in DI water for another three days. In both cases the dialysis solutions were changed once per day. The dry-state TEM sample preparation and TEM characterization methods were the same as described.

Synthesis of particles without TMB. The synthesis and TEM characterization methods used for particles without TMB were identical to those with TMB as described, except that the TMB addition step was omitted.

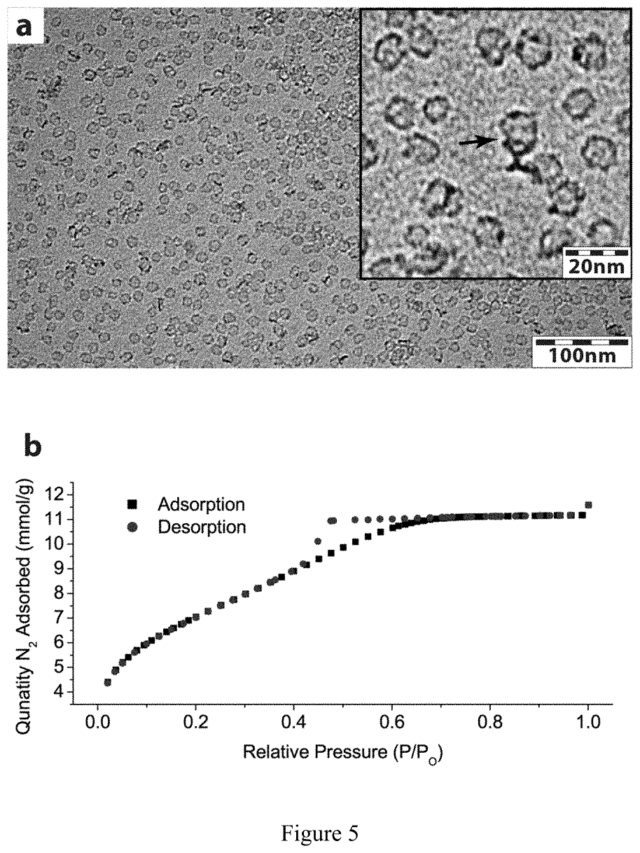

Silicage surface area. The specific surface area of the silicages was assessed by a combination of nitrogen sorption measurements and theoretical estimations. After PEGylated silicage synthesis and purification, particles were first up-concentrated using a spin filter (Vivaspin 20, MWCO 10 kDa) and dried at 60° C. Particles were then calcined at 550° C. for 6 hours in air. Nitrogen adsorption and desorption isotherms were acquired using a Micromeritics ASAP 2020 (FIG. 5b) yielding a specific surface area of 570 m²/g using the Brunauer-Emmett-Teller (BET) method. For comparison, using the dodecahedral cage model with the dimensions from the reconstruction shown in FIG. 3, a theoretical surface area of silicages was estimated to be around 790 m²/g. Overestimation of the experimental value is consistent with expected losses of surface area during sample calcination.

Additional details and optical characterization of the metal and vanadium oxide based syntheses of cage-like structures. In contrast to the sol-gel reaction leading to the silicage, the gold and silver cage-like structures syntheses relied on reduction reactions. To this end, tetrakis(hydroxymethyl)phosphonium chloride (THPC) was used as it reacts in water at basic pH to form trimethoxyphosphine, which can play both the role of reductant and capping agent for the metal nanoparticles. THPC has been widely used for the synthesis of ultra-small (<3 nm) and negatively charged phosphine-stabilized gold nanoparticles. These nanoparticles are often used as seeds for the subsequent growth of continuous gold shells on the surface of aminated silica nanoparticles thanks to their high affinity and binding efficiency with amine groups. Alcohol was added to the reaction mixture in order to mimic the conditions of the silicage synthesis where methanol is formed upon hydrolysis of TMOS. Early stage preliminary experiments showed that resulting structures were less size dispersed when using ethanol in slightly higher concentration than the released methanol in the silicage synthesis. Gold and silver cage-like structure syntheses were performed at a much lower concentration ([Au] or [Ag]=93.7 μM) as compared to the silicages ([Si]=65.9 mM). Attempts at synthesizing gold and silver cages at higher concentrations resulted in much larger nanoparticles with no apparent internal structure.

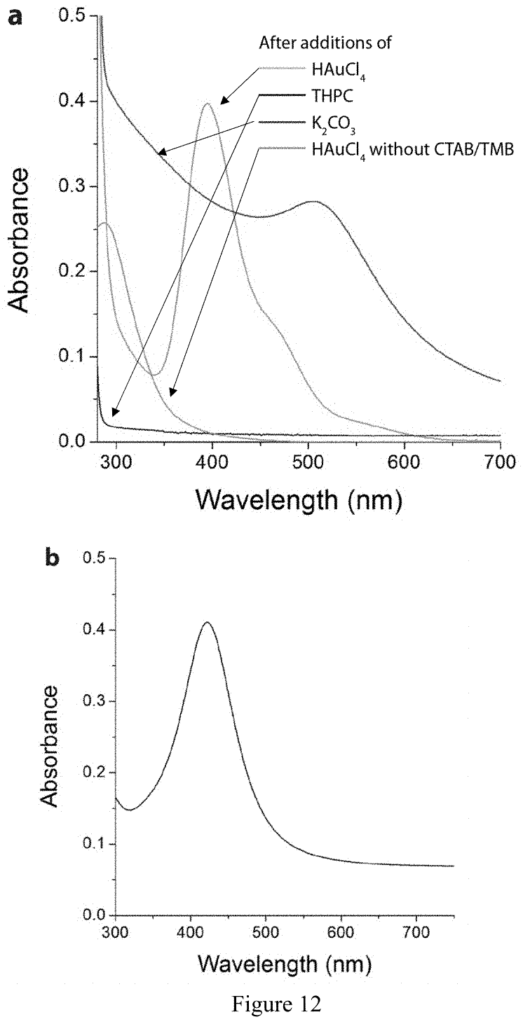

Gold based synthesis. The addition of gold precursor to the reaction initially resulted in the formation of a pale yellow precipitate which turned into a clear, i.e. non-turbid, darker orange solution within a couple of minutes under stirring at 30° C. (see FIG. 12 for a survey of the absorption characteristics at each step of the synthesis). Since neither the precipitate nor the darker orange coloration was observed in the absence of CTAB, these observations are attributed to some interaction between the gold chloride anions and the ammonium groups of the CTAB. After the addition of THPC, the solution turned colorless within a couple of minutes, indicating that gold(III) had been reduced to gold(I). The subsequent transformation of THPC into trimethoxyphosphine by increasing the pH with the addition of potassium carbonate happened within the first hour of reaction (see also description for the case of silver below). However, the reduction from gold(I) to gold(0) was found to be rather slow with the first hint of coloration appearing after 8 hours of reaction. After one day of reaction, the solution ended up exhibiting a brown coloration. This brown coloration was the signature of gold nanoparticles which are too small or not dense enough to show a strong surface plasmon resonance, as evidenced by the absorption profile in FIG. 12 which only shows a faint feature around 510 nm.

Silver based synthesis. The addition of silver precursor to the reaction did not initially translate into any visible effects, neither in the presence of CTAB/TMB nor after adding THPC. Nevertheless, after adding potassium carbonate to the silver based synthesis, the solution started to turn pale yellow within the first hour of reaction and resulted in an intense yellow coloration after 6 hours, at which point the TEM samples were prepared. This yellow coloration is classic for such small silver nanoparticles showing a surface plasmon resonance centered around 420 nm as shown in FIG. 12.

Vanadium oxide based synthesis. The vanadium oxide cage-like nanoparticles were prepared under the same conditions as the silicage, however the pH was not adjusted with ammonia due to the fast hydrolysis and condensation rate of the vanadium oxide precursor. In contrast to the metal cage-like nanoparticles synthesis, no alcohol was added here since the hydrolysis of the vanadium precursor, vanadium oxytriisopropoxide, produces alcohol similar to the silicage synthesis. The addition of this precursor to the TMB micelles resulted in the immediate formation of a red precipitate. Under stirring, the precipitate dispersed homogeneously in solution, which remained turbid, and turned orange after one day of reaction at 30° C.

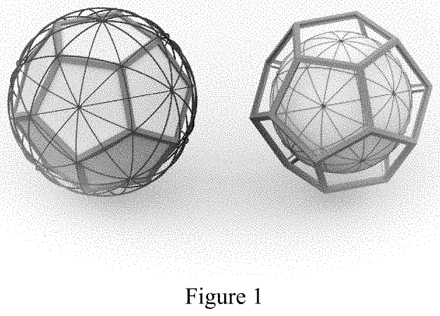

To produce dodecahedral silica cage structures (FIG. 1), the early formation stages of surfactant micelle directed silica self-assembly was investigated. The synthesis system contained cetyltrimethylammonium bromide (CTAB) surfactant micelles and tetramethyl orthosilicate (TMOS) as a sol-gel silica precursor. Hydrophobic mesitylene (TMB) was added into the aqueous CTAB micelle solution, increasing micelle size and deformability. TMOS was selected as the silica source due to its fast hydrolysis rate in water, and the initial reaction pH was adjusted to ˜8.5. Following TMOS addition, its hydrolysis to silicic acid reduced the reaction pH to neutral. The lowered pH accelerated silane condensation, forming primary silica clusters with diameter around 2 nm. The negatively charged silica clusters were attracted to the positively charged CTAB micelle surface, assembling into micelle templated nanostructures. This experimental design, where fast hydrolysis and condensation of the silica precursor quickly terminated the reaction process, allowed preservation of early formation stages of micelle directed silica self-assembly.

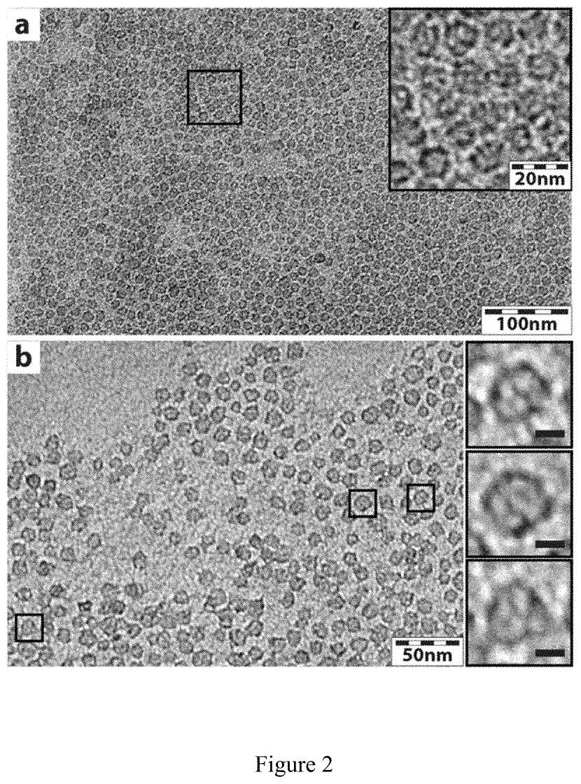

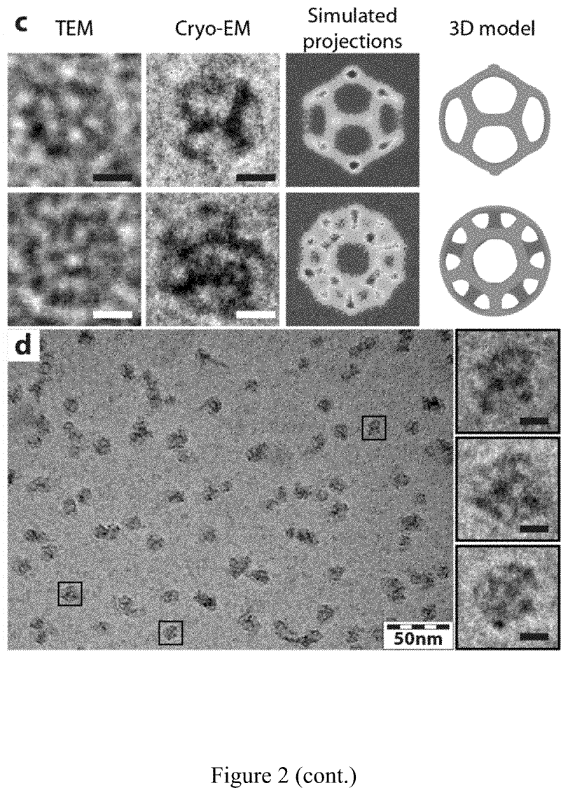

In order to improve particle dispersity on transmission electron microscopy (TEM) grids, low molar mass silane modified monofunctional polyethylene glycol (PEG) was added into the solution one day prior to TEM sample preparation, thereby covalently coating the accessible silica surface, yielding PEGylated nanoparticles (FIG. 5) that could be further purified and isolated from the synthesis solution. Narrowly size distributed particles were observed under TEM with average diameter around 12 nm (FIG. 2a and inset), consistent with silica structures wrapped around TMB swollen CTAB micelles. The detailed particle structure was difficult to identify, however. Therefore, TEM samples were subsequently plasma etched on carbon grids for five seconds prior to imaging to remove excess organic chemicals (e.g. PEG-silane), otherwise contributing to background noise. To further improve the signal-to-noise ratio, a series of images were acquired of the same sample area and averaged. Stripes and windows in zoomed-in images of individual particles became more clearly recognizable, suggesting the presence of cage-like structures (FIG. 2b and insets).

The study of thousands of such single particle TEM images revealed the prevalence of two cage projections with two- and, in particular, five-fold symmetry, respectively (FIG. 2c). Cryo-EM characterization was used to examine the native reaction solution. The silica surface PEGylation step was omitted as the high PEG concentration substantially increased radiation sensitivity of the samples, resulting in difficulties obtaining clear cryo-EM images.

Cryo-EM provided direct visualization of particles in solution with arbitrary orientation, i.e. without disturbances due to sample drying on TEM substrates, including structure deflation. The background noise was significantly reduced as a result of the absence of a TEM substrate as well as chemicals dried onto the substrate during sample preparation (FIG. 2c). Although particle aggregation was occasionally observed in cryo-EM (FIG. 2d), individual silica nanoparticles with cage-like structures could always be identified (FIGS. 2c and d). No particle aggregation was observed in dry-state TEM of PEGylated particles, suggesting that particle aggregation observed by cryo-EM was a reversible process that could be overcome via insertion of PEG chains.

˜19,000 single particle images were manually identified from cryo-EM micrographs, and they were clustered and the images averaged in each cluster in order to improve the signal-to-noise ratio. The averages showed different orientations of silica nanoparticles with cage-like structures, i.e. silicages (FIG. 6a). Averages were identified that were consistent with selected projections of a pentagonal dodecahedral cage (FIG. 2c and FIG. 6b). The dodecahedral silicage (icosahedral point group, Ih, FIG. 1) is the simplest of a set of Voronoi polyhedra suggested to form the smallest structural units of multiple forms of mesoporous silica. Although such highly symmetric ultrasmall silica cages have never been isolated before, it seemed likely that this should be possible.

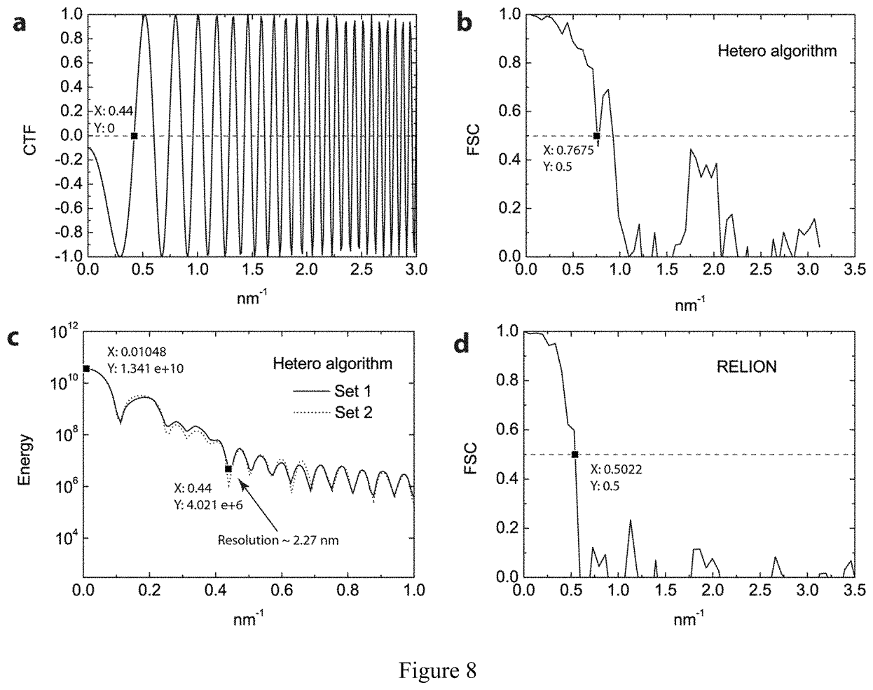

Guided by this structural insight, single-particle 3D reconstruction of silicages were performed using the “Hetero” model-based maximum likelihood algorithm, in which a two-class reconstruction was computed to overcome challenges associated with structural heterogeneity and rotational icosahedral symmetry was imposed on both classes (FIG. 6). One of the two-class reconstructions was a dodecahedral cage (FIGS. 3a and b). Low intensity signal was identified inside the reconstructed cage, consistent with the presence of TMB swollen CTAB micelles inside the silicage, whose electron density is lower than silica but higher than the surrounding ice. The other class (i.e., non-cage) did not provide an interpretable structure, likely due to heterogeneity in the structure of the corresponding particles. Such two-class reconstructions were performed using different numbers of single particle images (2000, 7000, and 10000) and yielded consistent results. Single-class reconstructions were also performed, using only the images in the class showing dodecahedral cages in two-class reconstructions, by the Hetero algorithm. Equivalent two-class and single-class reconstructions were also performed by the widely-used RELION 2.1 system. Dodecahedral cage structures were obtained in all these reconstructions (FIG. 7). The resolution of the reconstructions was approximately 2 nm (FIG. 8). Silica in these cages is amorphous at the atomic level, which prevented atomic resolution in these reconstructions.

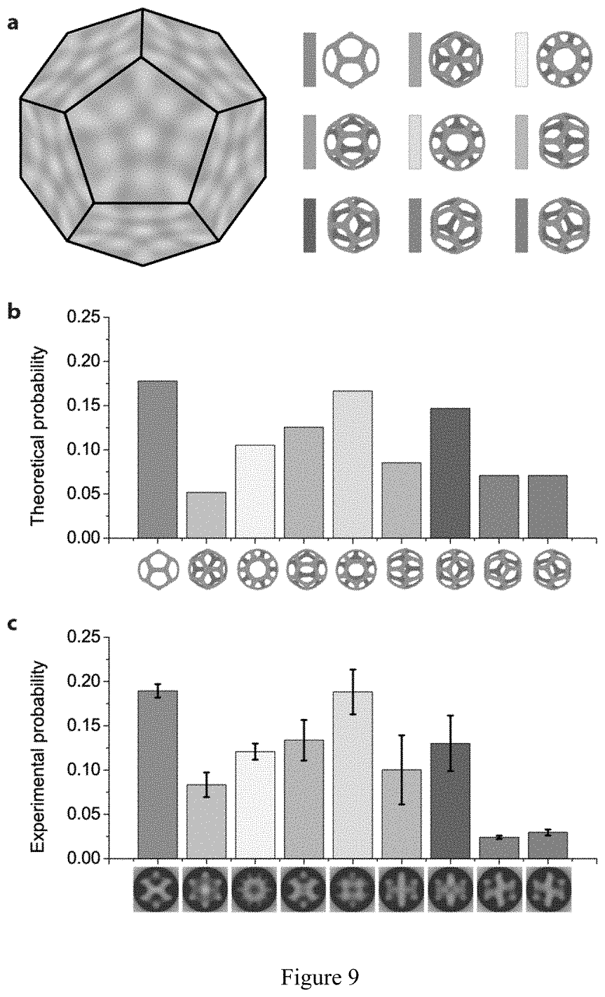

The Hetero reconstruction algorithm provided estimates of the projected orientation (i.e., three Euler angles) for each experimental image, which were used to compute predicted projections. Nine predicted projections and corresponding experimental images were manually clustered, and averages were computed for each cluster (FIG. 3c). The similarity of the projections of the 3D reconstruction and the averaged experimental images supports the dodecahedral cage structure. Furthermore, the theoretical probabilities of finding each of the nine projections (FIG. 3c) were calculated based on the assumption that the orientation of silicages in cryo-EM are random. The results were then compared to the probabilities observed by single particle 3D reconstruction (FIG. 9). The high consistency between theoretical and experimental projection probabilities further supports the dodecahedral cage reconstruction.

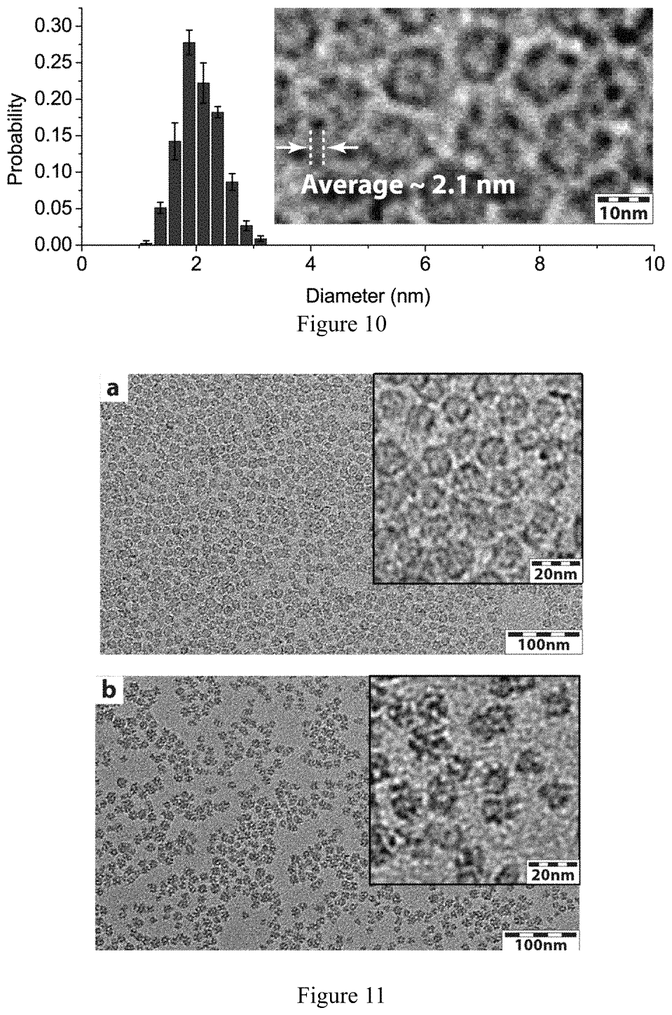

The vertices of the dodecahedral silicage had a diameter around 2.4 nm (FIG. 3b), only slightly larger than the diameter of primary silica clusters, i.e. ˜2 nm (FIG. 10). The interstitial spacing between two nearby vertices was estimated to be about 1.4 nm (i.e., edge length, 3.8 nm, minus diameter of vertices, 2.4 nm, see FIG. 3b), much smaller than the diameter of such clusters. Bridges between vertices forming the edges of the dodecahedron were substantially thinner than the size of the primary clusters (FIGS. 3a and b). This suggests that negatively charged primary silica clusters formed in solution may start to come down onto the positively charged micelle surface attracted by Coulomb interactions. As more and more silica clusters assemble on the micelle surface, as a result of their repulsive interactions and possible interactions with other micelles, they may move to the vertices of a dodecahedron. Additional silane condensation onto the surface of growing clusters may eventually lead to bridge formation resulting in the final observed cage structure (FIG. 3). The origin of icosahedral symmetry in viruses has been associated with the energy minimization of two opposing interactions, repulsive interactions associated with the bending rigidity and attractive hydrophobic interactions. In a related way, in addition to electrostatic interactions, deformation of the micelle surface around the silica clusters may be another important contributor to the free energy in the system. This is supported by experiments showing that the cage structures do not form in the absence of TMB (FIG. 11), which is expected to enhance micelle surface deformability.

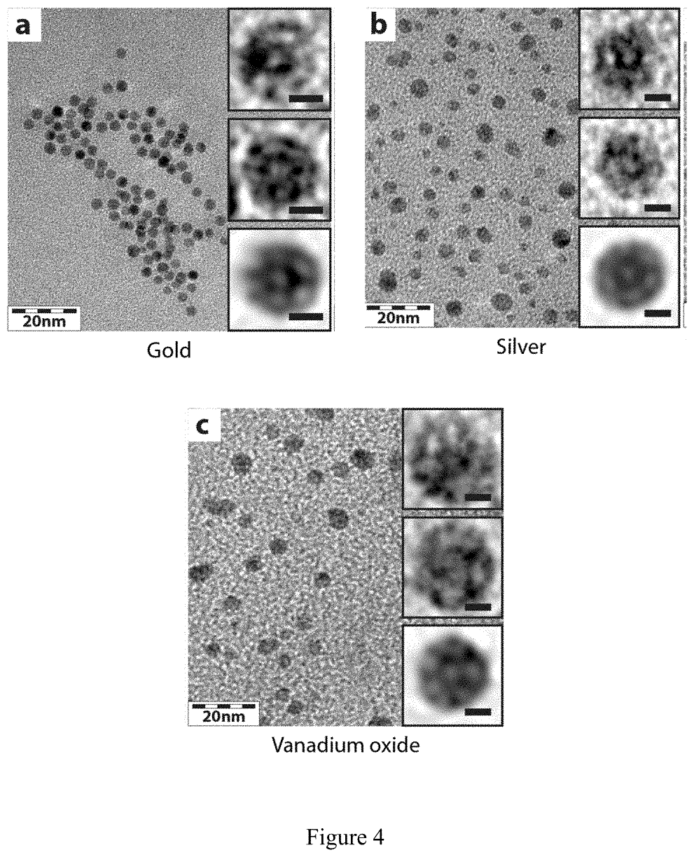

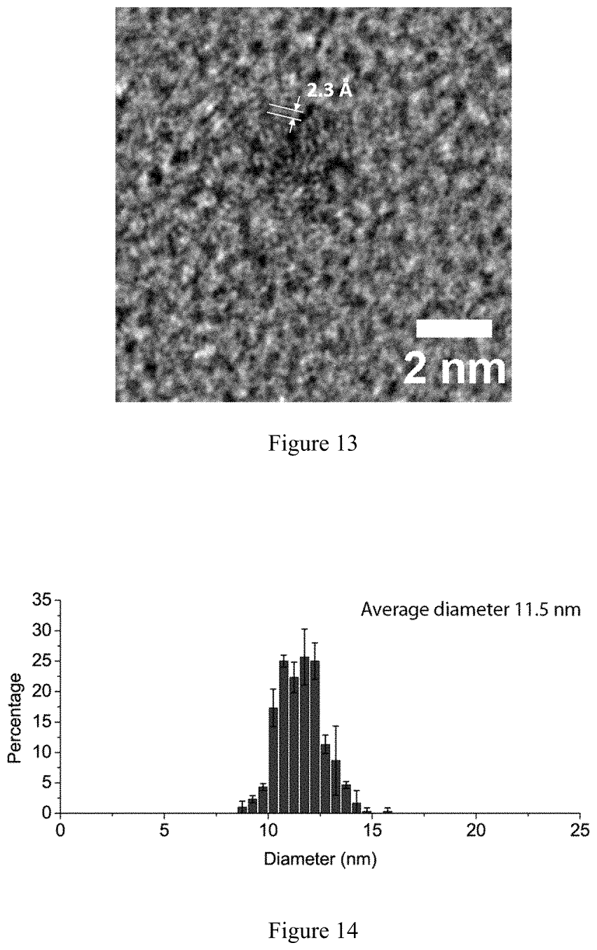

Micelle self-assembly directed ultrasmall cage structures could also be fabricated from other inorganic materials with similar feature sizes and surface chemistry characteristics to silica. In preliminary experiments silica was replaced by two metals, gold and silver, and a transition metal oxide, vanadium oxide. Gold and silver structures were prepared by the reduction of metal precursors, HAuCl₄.3H₂O and AgNO₃, respectively, in the presence of the micelles (FIG. 12). Tetrakis(hydroxymethyl)phosphonium chloride (THPC) was used as both the reductant and the capping agent to stabilize primary gold and silver nanoparticles and provide negative surface charges. In contrast, primary vanadium oxide nanoparticles with native negatively charged particle surface were prepared via sol-gel chemistry, similar to the synthesis of silicages. Images of individual particles obtained by TEM revealed similar internal structure (FIG. 4). These nanoparticles did not appear to be dense but instead showed cage-like structures (compare FIGS. 2 and 4), further corroborated by associated projection averages revealing cages with rotational symmetry (bottom insets in FIG. 4), similar to the prevalent projection in case of the silicage (vide supra). Micelle self-assembly directed cages like the dodecagonal structure described in this paper may therefore not be unique to amorphous silica, but may provide direct synthesis pathways to crystalline material cages (FIG. 13).

The unique structure of silicages renders them a promising novel material platform useful for applications ranging from nanomedicine to catalysis.

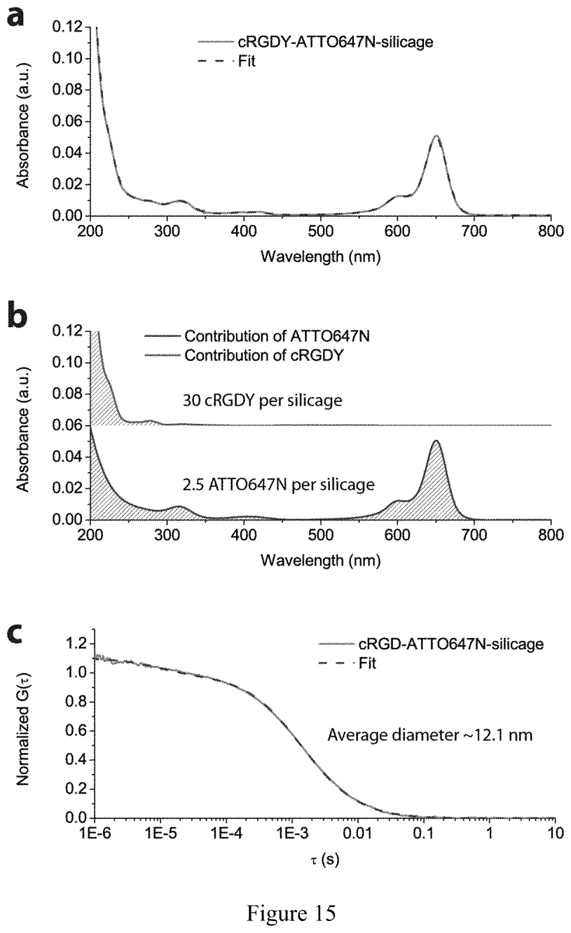

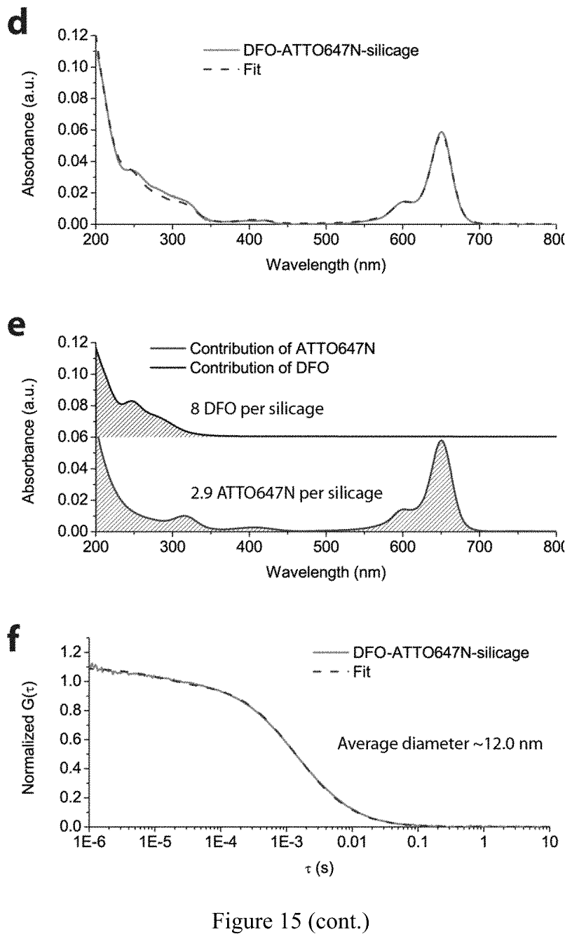

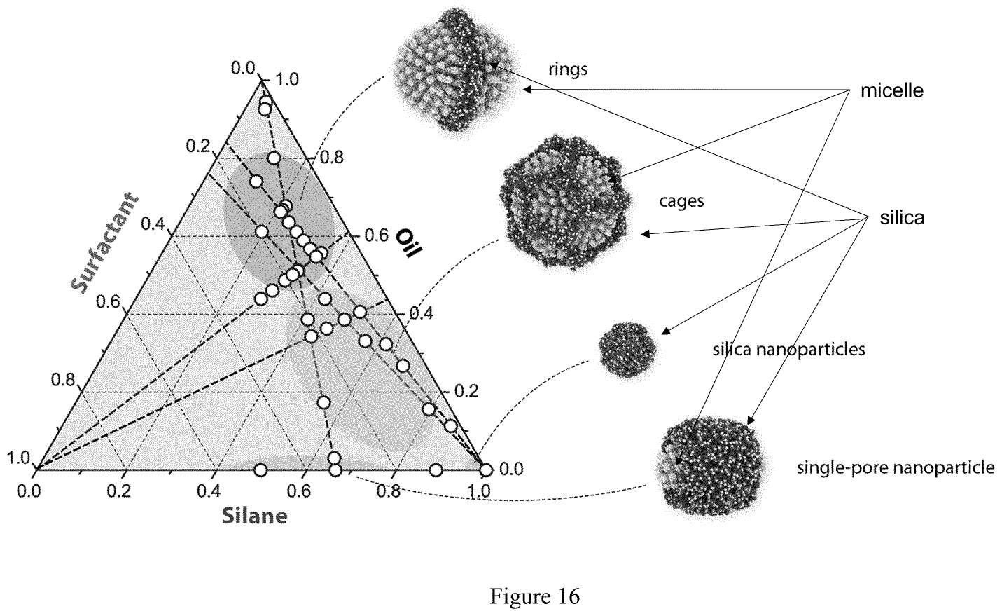

For example, as descripted, the silicages can be PEGylated by covalently attaching polyethylene glycol (PEG) to the outer cage surface, which substantially improves their bio-compatibility for bio-medical applications. While keeping the overall particle size slightly above 10 nm (FIG. 14), important for renal clearance, the fully empty interior of the PEGylated silicages after cleaning provides a cavity with large volume for the loading/release of small drug molecules for drug delivery. Since the silicages are PEGylated when the inside of the cage is occupied by surfactant micelle, the inner cage surface can be selectively modified after PEGylation with specific functional groups to facilitate drug loading/release. Additionally, by co-condensing dye-silane conjugates with TMOS in the step of silica formation, fluorescent dyes can be covalently encapsulated into the silica matrix of silicages, endowing silicages with fluorescence properties for tracing particles, as well as for imaging applications (FIG. 15). The dyes, which can be encapsulated into silica, include, but are not limited to, near-infrared ATTO647N. Furthermore, the outer surface of silicages can be modified with a type of, or a combination of different types of, functional groups by covalently attaching ligand groups to silicage surface during or after the PEGylation step. The functional ligands, which can be attached to the outer surface of silicages, include, but are not limited to, cancer targeting peptides (FIG. 15a to c), chelators of radioisotopes (FIG. 15d to e), DNAs, RNAs, antibodies, and antibody fragments. The multi-functional PEGylated silicages have significate application potential in the field of disease diagnosis and drug delivery.

Synthesis of cRGDY-ATTO647N-silicages and DFO-ATTO647N-silicages. To encapsulate fluorescent dyes into the silica matrix of silicages, dye-silane conjugates, e.g. ATTO647N-silane, were added together with TMOS at the beginning of the synthesis reaction, while the rest of the synthesis remained the same as the synthesis of PEGylated silicages which is earlier described.

To surface functionalize silicages with different types of functional ligands, ligand-silane conjugates, e.g. cRGDY-PEG-silane, were added into the reaction mixture right before the addition of PEG-silane in the PEGylation step. The rest of the synthesis remained the same as that of PEGylated silicages which is described earlier.

An alternative method to surface functionalize silicages with different types of functional ligands is via post-PEGylation surface modification by insertion (PPSMI) described in an earlier study. For example, PEGylated silicages are synthesized. After that and before particle purification, (3-aminopropyl)trimethoxysilane (amine-silane) is added into the reaction solution to covalently attach the amine groups onto the silica surface by inserting them between the PEG chains via condensation with silica surface silanol groups. Following that, isothiocyanate conjugated ligands, e.g. deferoxamine (DFO, one of the most efficient chelators for radio-labeling with Zr⁸⁹) in the form of DFO-isothiocyanate conjugate, are then added into the reaction solution to further covalently attach onto the cage surface via amine-isothiocyanate conjugation reaction. The rest of the synthesis, including the particle purification, remains the same as that of PEGylated silicages.

Inorganic nanocages of this application are highly-symmetric, including, but not limited dodecagonal cages. Other geometries include, but are not limited to, icosahedral, cubic, hexanol, tetrahedron, octahedral, and buckyball-like cages. The inorganic nanocages have an empty interior. The cage surface contains open windows connecting the inside and outside. The inorganic nanocages have particle size <30 nm. The inorganic nanocages can have a composition of only silica, and/or other inorganics, including, but not limited gold, silver, and vanadium oxide.

Method of synthesis of silica nanocages uses TMOS (or other inorganic precursors), CTAB, TMB. It also uses TMOS as the silica source of silica nanocages. The concentration of NH₃—H₂O is less than or equal to 10 mM. The low NH₃—H₂O concentration, as well as the associated low pH, substantially increase the condensation rate of TOMS. Thus, the reaction kinetics can be adjusted to just the right point to trigger the unique self-assembly of inorganic nanocages.

### Example 2

The following example provides a description of administration of nanorings of the present disclosure.

Described is the biodistribution in mice of silica nanomaterials around 10 nm in size with four different topologies: spheres, hollow beads, cages, and rings. In contrast to regular spherical particles, whose uptake in organs (e.g., liver, spleen) of the reticuloendothelial system (RES) increases with increasing diameter, for this sequence, record low RES uptake with increasing size was surprisingly observed. Rings get effectively cleared via the kidneys for diameters larger than 15 nm, i.e., well above the cut-off for renal clearance around 6 nm. Results suggest that topology is a hitherto neglected parameter in materials design for applications in nanomedicine, enabling low RES uptake and efficient renal clearance for object diameters well above 10 nm.

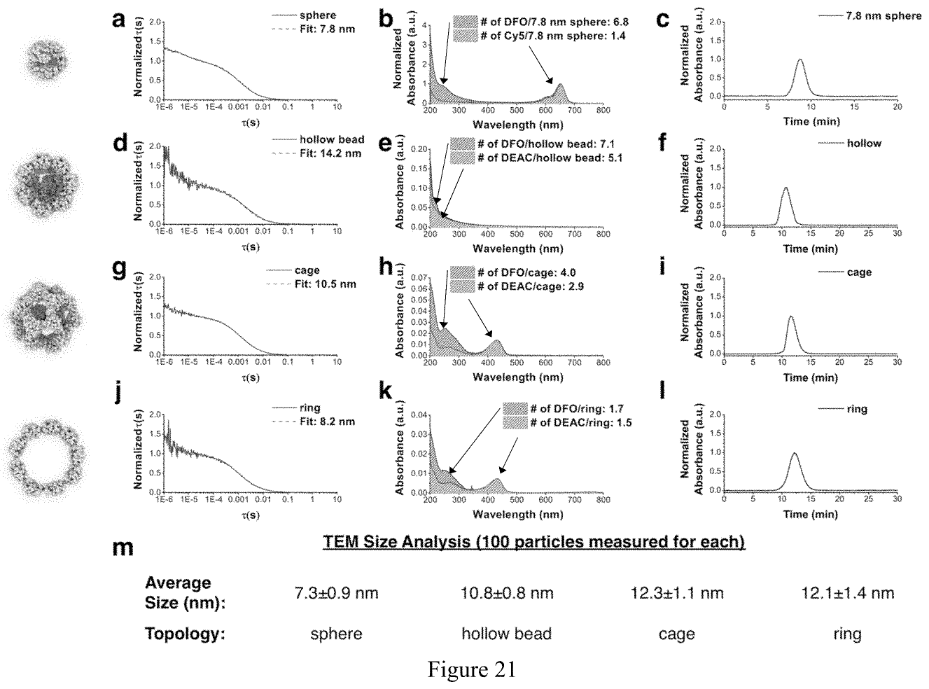

Silica nanoparticles (NPs) with ˜10 nm diameter were synthesized from tetramethyl orthosilicate (TMOS), cetyl-trimethylammonium bromide (CTAB), and 1,3,5-trimethylbenzene (mesitylene, TMB) in aqueous solutions as a way to keep structural parameters, other than topology (e.g., size, shape, surface chemistry, surface charge), similar across all particles. NP topology was engineered by adjusting CTAB and TMB concentrations. In their absence, ˜4 nm diameter spherically shaped silica cores were formed. When TMB swollen CTAB micelles were introduced, ˜2 nm-sized primary silica clusters self-assembled on their surfaces, leading to the formation of silica rings, cages, or hollow beads depending on reagent ratios (Methods). Dyes endowed the particles with fluorescence (Methods), while poly(ethylene glycol) (PEG) coatings (Methods) provided for steric stability and improved biocompatibility. Deferoxamine (DFO) was attached onto all particle surfaces as a chelator for zirconium-89 (⁸⁹Zr, t_{1/2}=78.4 h), enabling quantitative serial positron emission tomography (PET) imaging and biodistribution analyses (Methods). Particles were purified by gel permeation chromatography (GPC) and compositions characterized before final use (FIG. 21).

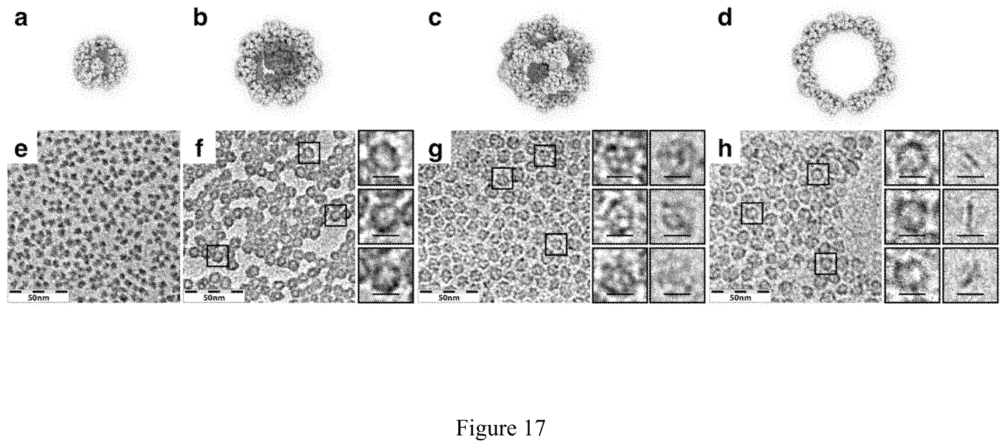

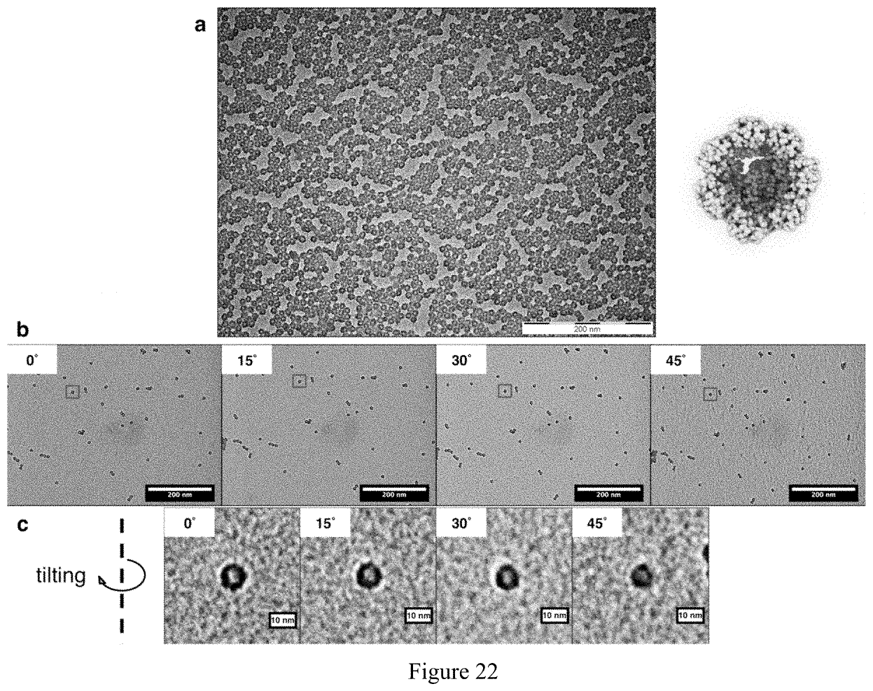

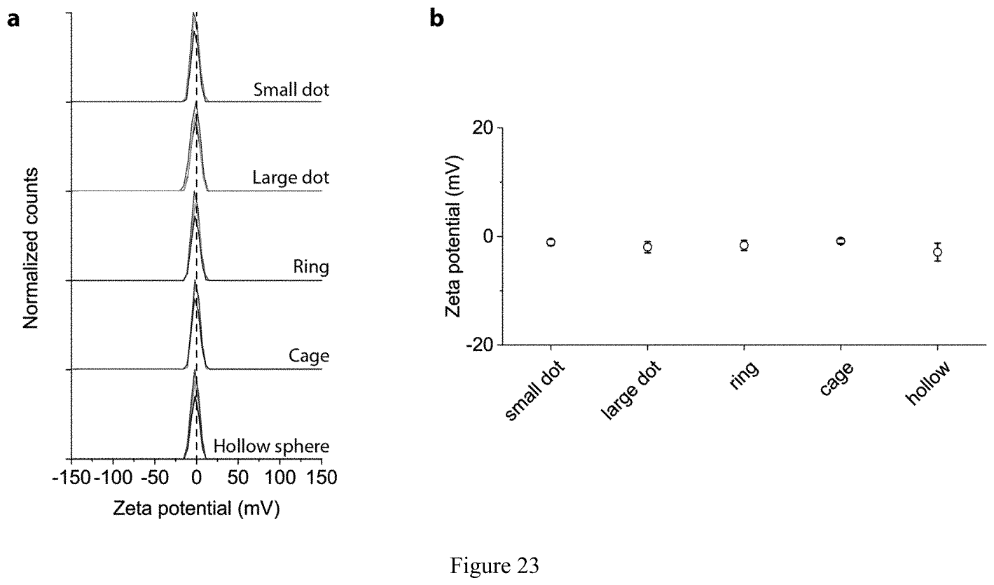

Hydrodynamic (or equivalent hydrodynamic) particle diameters (Methods) were determined using fluorescence correlation spectroscopy (FCS), while particle topology and silica core diameters were characterized by transmission and cryogenic electron microscopy (TEM, cryo-EM). The larger size of hollow beads, cages, and rings relative to spheres was easily discerned (FIG. 17), while detailed inspection (see insets FIG. 17) revealed established features and projections consistent with cage and ring topologies. The structure of hollow beads formed around CTAB micelles was confirmed with a TEM tilt series (FIG. 22). Diameters measured by TEM for spheres, beads, cages, and rings were 7.3 nm, 10.8 nm, 12.3 nm and 12.1 nm, while their (equivalent) hydrodynamic FCS sizes were 7.8 nm, 14.2 nm, 10.5 nm, and 8.2 nm, respectively (FIG. 21). While for spherical and hollow particles FCS provides a larger diameter than TEM owing to PEG and dragged water shells, it underestimates the diameters of cages and rings due to the assumption of a spherical shape in the model-based analysis (Methods). Zeta-potential measurements for all particles showed values close to zero, consistent with successful PEGylation (FIG. 23).

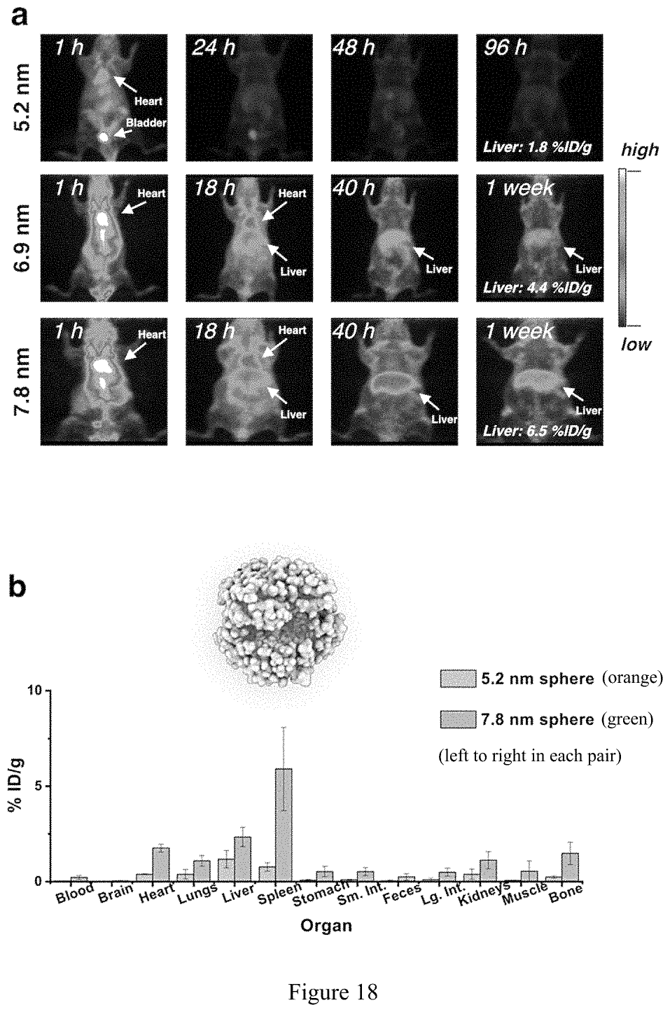

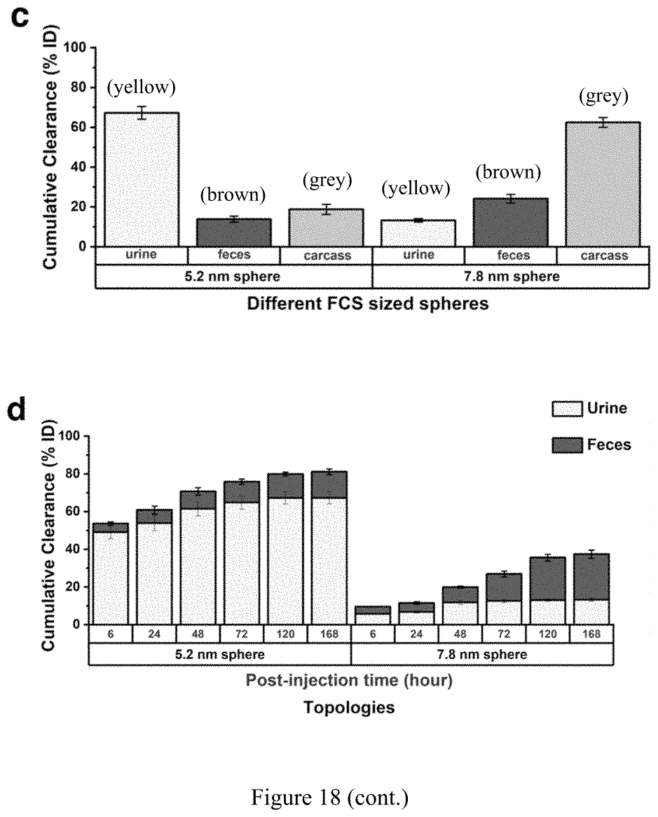

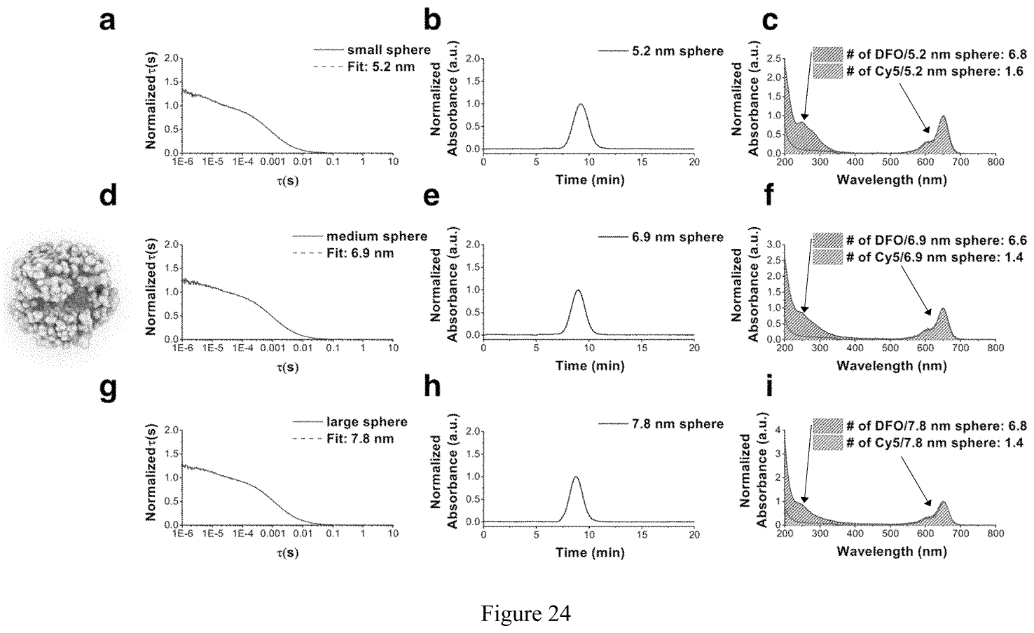

NP biodistribution is typically dependent on diameter below 10 nm; e.g., liver uptake substantially increases with increasing particle size, while the ability to clear via the kidneys diminishes. To illustrate this behavior, spherical dots with 5.2 nm, 6.9 nm and 7.8 nm hydrodynamic (FCS) diameters (FIG. 24) were radiolabelled with ⁸⁹Zr. These particle tracers were intravenously (i.v.) injected into healthy nude mice. Serial PET scans were acquired over a 1-week period (Methods) to study time-dependent particle pharmacokinetics (PK) and whole-body biodistribution. From selected coronal PET images (maximum intensity projections, MIPs, FIG. 18a), liver uptake was found to increase from 1.8 to 4.4 to 6.5% ID/g. Ex vivo biodistribution studies were performed 1 week after i.v. injection to quantitatively evaluate organ/tissue-specific uptake of small (5.2 nm) and larger-size (7.8 nm) particle tracers, respectively (Methods). Similar to findings on PET, as dot size increased, mean tissue-specific uptake values went up in the heart (blood pool) and kidneys, as well as in organs of the RES (FIG. 18b), namely the spleen (˜0.8 to 6% ID/g), liver (˜1.2 to 2.3% ID/g), bone marrow (˜0.2 to 1.5% ID/g), and lungs (˜0.4 to 1.1% ID/g). Organ-specific differences were statistically significant (p<0.001). Time-dependent particle tracer activities in urinary and fecal biological specimens were monitored using a metabolic cage set-up (Methods) following i.v.-injection of small and large spheres. At 1 week post-injection (p.i.), cumulative urinary clearance (% ID, FIG. 18c) exhibited a substantial drop from around 67 to 13% ID as particle size increased from 5.2 nm to 7.8 nm, whereas a rise in fecal clearance was observed (i.e., ˜14 to 24% ID). Retained activity, i.e., dots remaining in the carcass, accounted for about 19 and 63% ID for 5.2 nm and 7.8 nm particles, respectively, suggesting ˜3 times less total clearance for the larger dots. Adjusting for these different clearance routes, statistically significant differences (p<0.001) were found between particle sizes. In time-dependent clearance profiles from metabolic cage studies up to 1 week p.i. (FIG. 18d), while the urinary clearance of 5.2 nm dots was nearly 50% ID at 6 hours p.i., order of magnitude lower urinary clearance was seen for 7.8 nm dots at a similar p.i. time. Statistical significance was achieved for both cumulative urinary clearance at 168 hours p.i. (p<0.001), as well as for the rate of accumulation (p=0.017) across particle sizes.

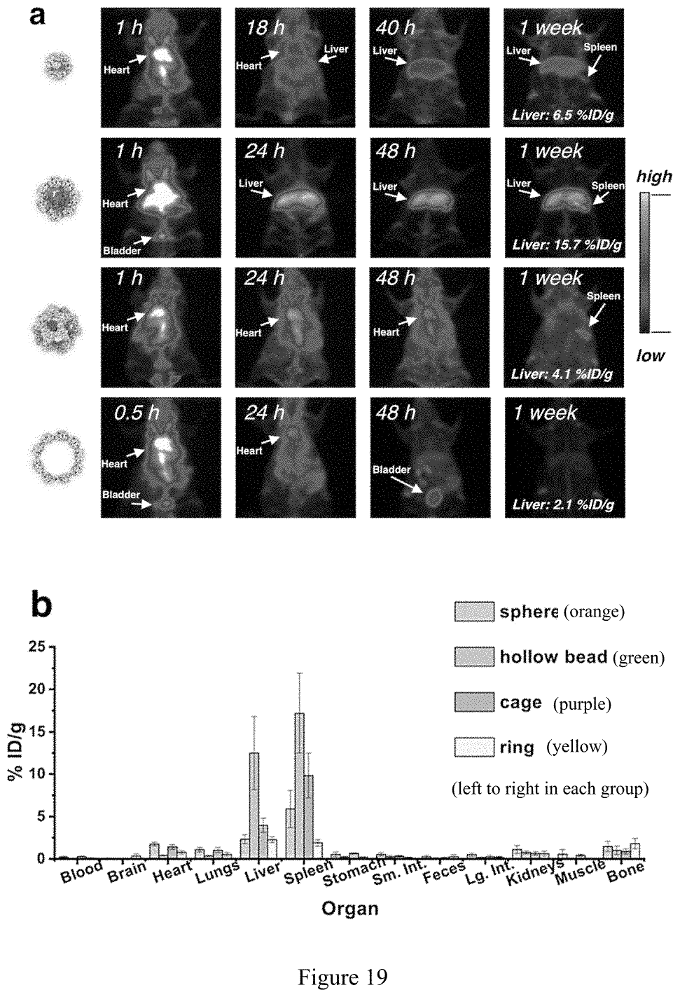

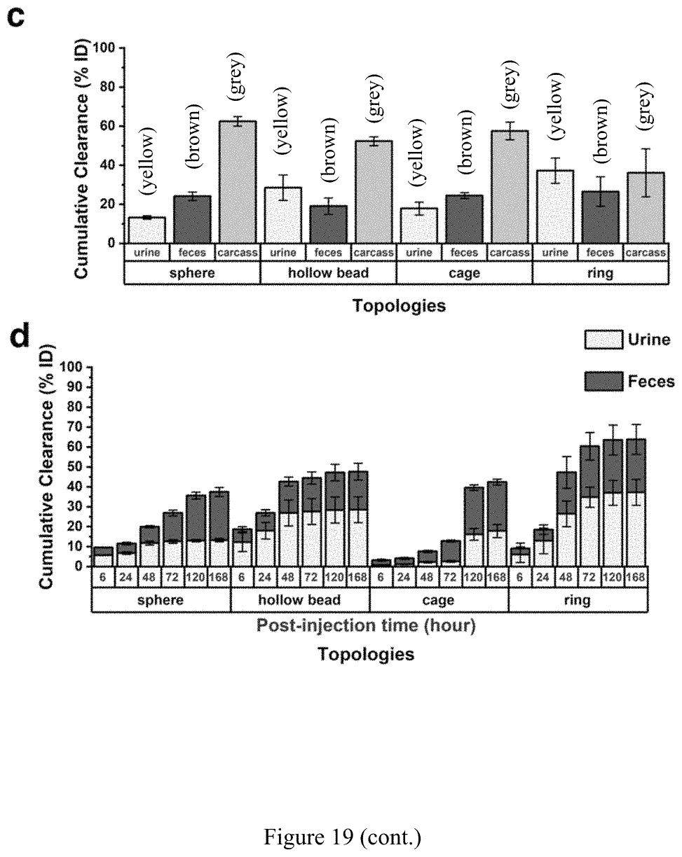

Observations of progressively higher RES uptake with concomitant decreases in renal excretion as particle size increases are consistent with prior studies. Surprisingly, however, these trends were inverted when moving to objects with even larger sizes, but different topologies in the form of hollow beads, cages, and rings measuring 10.8 nm, 12.3 nm, and 12.1 nm (TEM) in diameter, respectively. Results of serial PET imaging and biodistribution studies up to 1 week p.i. in healthy mice after ⁸⁹Zr radiolabeling and i.v. particle injection are compared to the PK profile of the 7.3 nm (TEM) diameter dots in FIG. 19. At early p.i. time points (i.e., ˜1 hour), high particle tracer activities were observed in the heart and liver for all topologies, as expected, consistent with higher vascular perfusion to these organs. By 40-48 hours, however, cardiac activities had substantially decreased from that seen at 18-24 hours across all topologies, except for cages. Regarding clearance properties, bladder activity was already detectable on MIP images for hollow beads and rings at early time-points (FIG. 19a, col 1), while hepatic activity became apparent at ˜24 hours p.i. for hollow beads and spheres. At 1 week p.i., analysis of hepatic activity for each topology was derived from the individual coronal tomographic images acquired. Hollow beads were noted to exhibit maximum hepatic uptake values of 15.7% ID/g, followed by values of 6.5% ID/g for spheres, 4.1% ID/g for cages, and 2.1% ID/g for rings (scale bar, FIG. 19a). The value of 2.1% ID/g for rings is the lowest reported to date for such silica NPs with diameters above 10 nm. Moreover, rings did not demonstrate any appreciable splenic uptake at 1 week p.i., while splenic uptake (arrows) was observed for spheres, hollow beads, and cages. While increased hepatic and splenic activities were initially noted moving from a dot size of 7.3 nm to a hollow bead size of 11 nm, these results contrasted with a relative lack of observable activities in these organs for larger-sized (i.e., ˜12 nm) cages and rings.

In ex vivo biodistribution studies, each of the four topologies was evaluated at 1 week p.i. of radiolabeled particles (FIG. 19b). Results were consistent with those found at 1 week on serial PET imaging (FIG. 19a). As particles transitioned from 7.3 nm dots to 10.8 nm hollow beads, approximately 5-fold and 3-fold increases in hepatic and splenic uptake were observed, respectively (FIG. 19b). Intriguingly, at even larger particle sizes, substantial decreases in hepatic and splenic activity were noted for both 12.3 nm cages and 12.1 nm rings. Specifically, relative to hollow beads, cages exhibited approximately 3-fold and 1.7-fold drops in hepatic and renal activity, respectively, while rings exhibited even larger fold changes of 5.5 and 9 for these activities, respectively (FIG. 19b). Results were statistically significant across all topologies (p<0.001), adjusting for different organs.

Metabolic cage studies performed on the four topologies (FIG. 19c) showed at 1 week p.i that 7.3 nm dots were associated with the lowest urinary and total clearances (i.e., ˜13 and 38% ID, respectively), while rings exhibited the highest (i.e., ˜38 and 64% ID, respectively). Results were statistically significant (p<0.0001) across the four topologies. Time-dependent clearance studies (FIG. 19d) provided a more differentiated picture. Cumulative (total) clearances (% ID) increased from 6 to 168 hours, but were surprisingly delayed for both cage and ring samples. In particular, for cages, total urinary and fecal clearance did not substantially increase until about day 5 p.i., noting a 13-fold increase relative to early time points (i.e., 6 hours). Statistical significance was established among topologies for total urinary clearance (p<0.0001) and rates of accumulation (p=0.0001). At later times p.i., relative contributions of both urinary and fecal excretion became fairly equivalent for both cages and spheres (FIG. 19d). Urinary excretion for both rings and beads looked fairly equivalent at later time points.

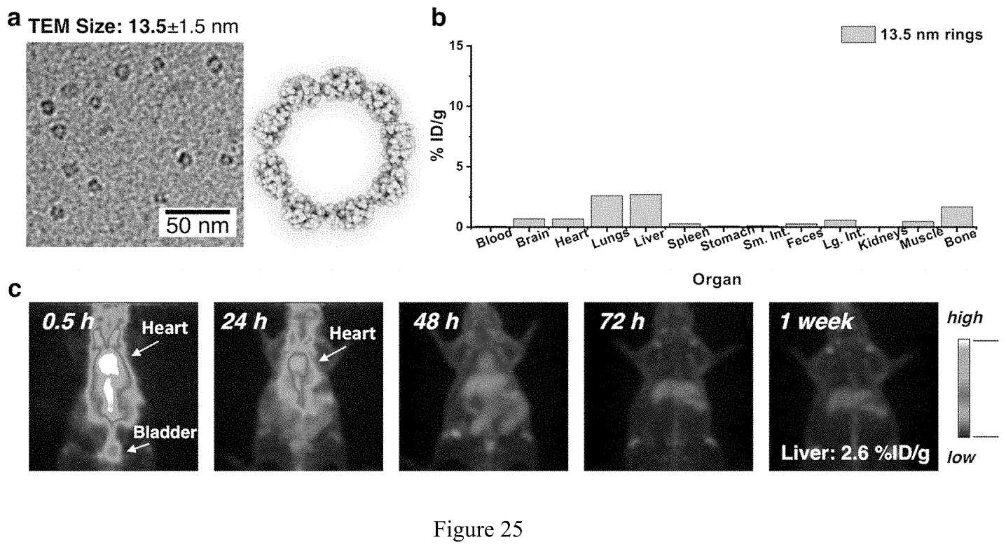

Spheres, hollow beads, cages, and rings have very different topologies, i.e., there are no simple continuous deformations that can transform these geometrical objects into each other without tearing holes (i.e., they are not homeomorphic). In nature, protein structures with ring or cage topologies are ubiquitous and play crucial roles, e.g., in cellular function. For the first time, a set of inorganic nanoobjects with these varying topologies was synthesized, but otherwise similar shapes and surface chemical properties, as well as sizes around 10 nm (see FIG. 21), in order to study the effects of topology on biological response. While increases in the diameter of spherical silica NPs led to significant, but expected, increases in RES uptake and decreases in cumulative urinary clearance, the opposite trend was observed for the largest diameter objects, in particular for rings (also see FIG. 25). It is proposed that topology dependent properties, i.e., deformability in case of urinary excretion and diffusivity in case of RES uptake, can rationalize these surprising observations.

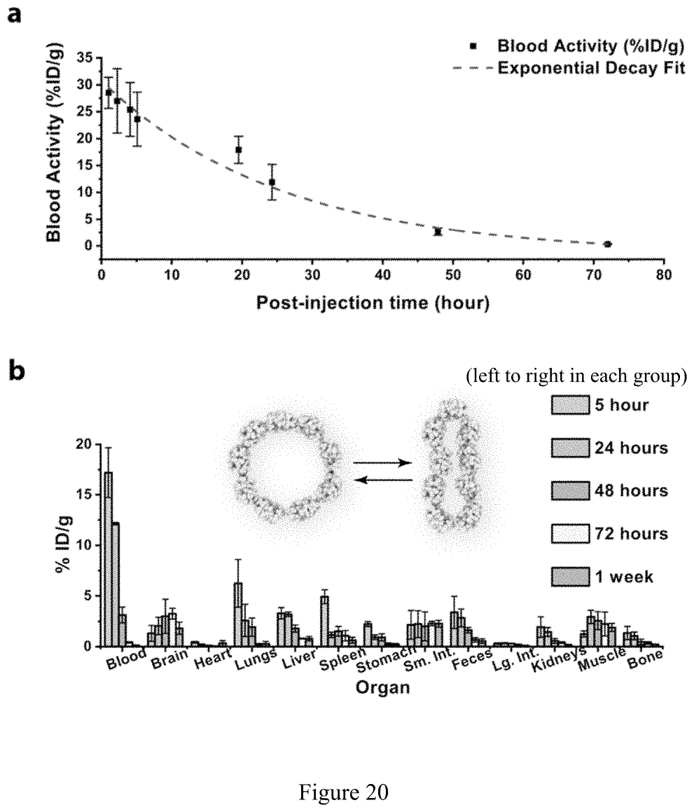

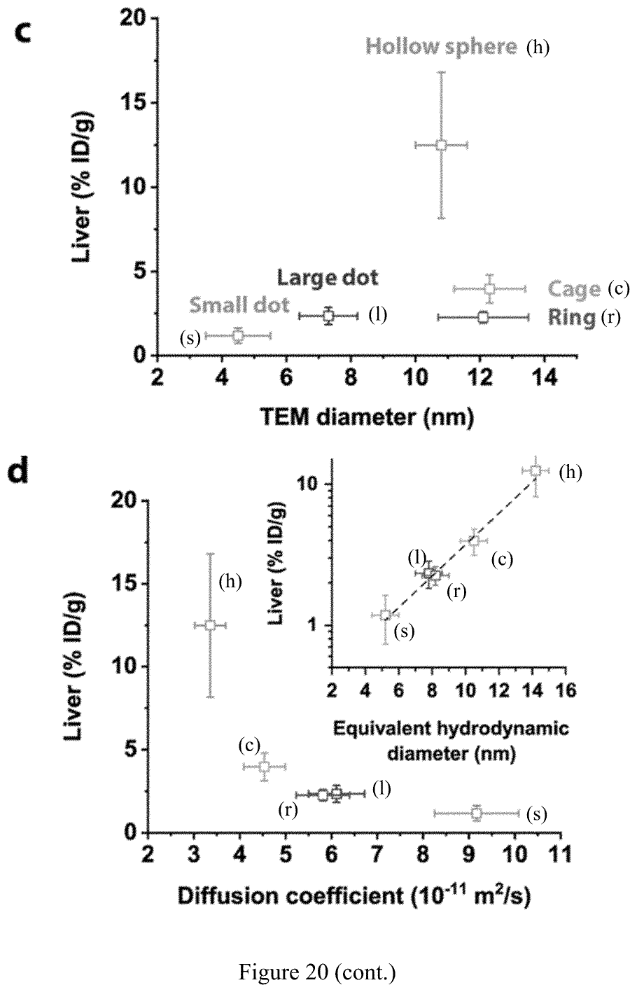

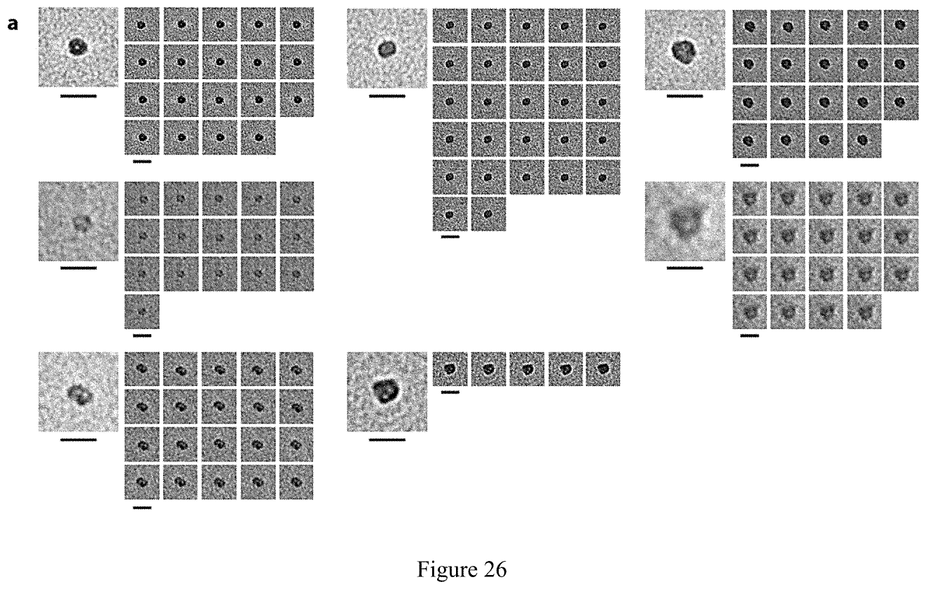

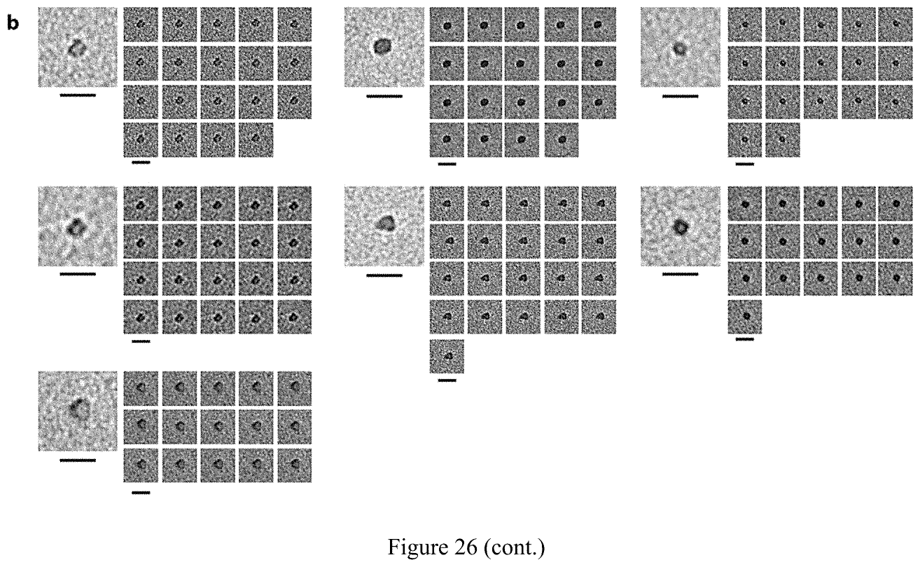

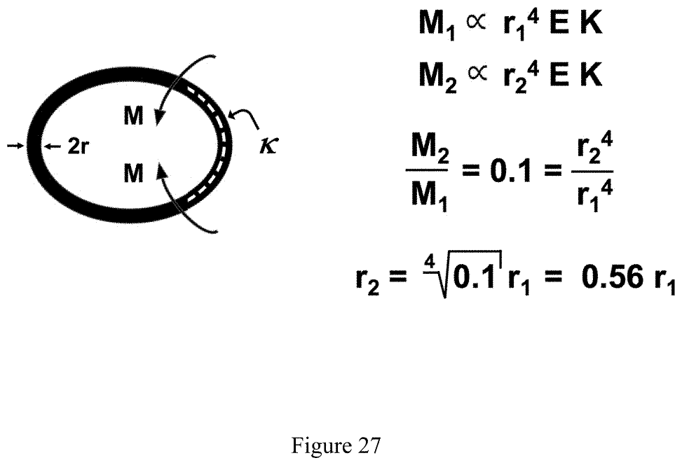

An explanation for the renal clearance of hollow beads, cages and rings with sizes well above the effective renal glomerular filtration size cut-off for inorganic NPs around 6 nm could be their degradation through, e.g., shear forces, with resulting smaller pieces clearing out. We verified, however (FIG. 26), that these objects cleared without degradation, by collecting urine from mice at 2-hour p.i. and TEM analysis (Methods) for cage and ring topologies (expected to be particularly prone to this mechanism). Such amorphous silica NPs can deform as a result of their structural elements, i.e., ˜2 nm diameter primary silica clusters, connected via thin bridges into shells of hollow beads, struts and vertices of cages, and the backbone of rings. At small length scales, even crystalline materials are flexible. Despite their size, indeed model calculations suggest (Methods, FIG. 27) that they can undergo glomerular filtration in the kidneys by being “squeezed” by the glomerular capillary pressure (FIG. 20b, inset). Deformations are facilitated by a “pearl-chain” type structure, where bending is localized to the thin and compliant bridges connecting the silica clusters. Fully squeezing rings together, the combined diameter of the two silica struts next to each other, is ˜4 nm, i.e., below the cut-off for renal clearance.

The concept of topology dependent inorganic NP deformation is further supported by the ring blood circulation half-life, t_{1/2}=17.8 h (FIG. 20a), which is longer than that of smaller dots, 15.3 h for 6.5 nm dots, with similarly low liver uptake (<5% ID/g). Rings undergo glomerular filtration when they get squeezed, which takes longer. Rings also show higher clearance via feces as compared to smaller (5.2 nm) dots, 27% vs 14% (FIG. 18c-19c), respectively. As hepatic clearance takes longer than renal clearance, this is consistent with the increased blood circulation half-life of rings. For example, we measured a blood activity of 12% ID/g for rings at 24-hour p.i. (FIG. 20a), much higher than that of the dots (highest blood-activity of 6% ID/g at 24-hour p.i.). Results of time-dependent biodistribution studies performed for rings reveal no significant uptake by RES organs, even at early time points (FIG. 20b). Blood activity decreases significantly at 48-hour p.i., consistent with significant renal and hepatic clearance for this time-point in time-dependent metabolic cage studies (FIG. 19d).

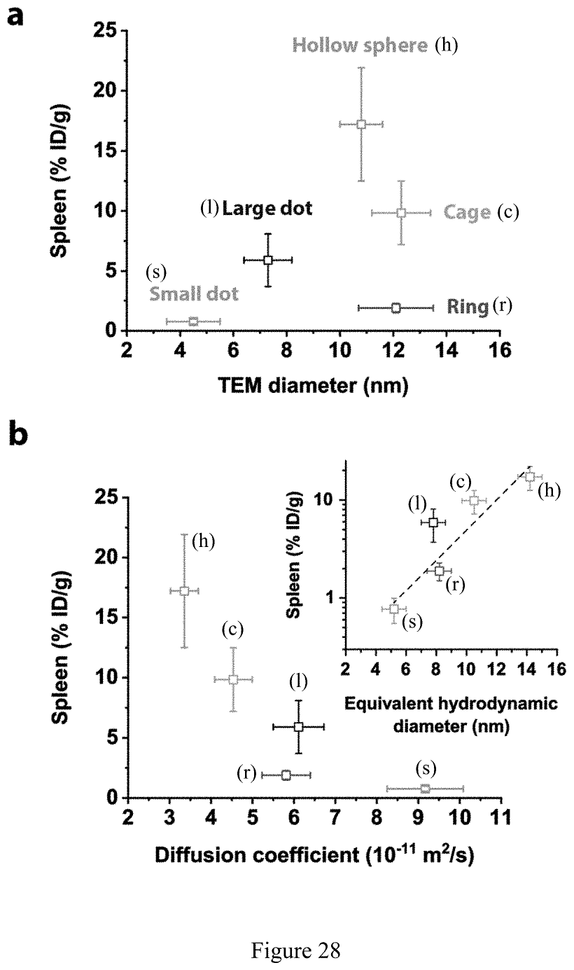

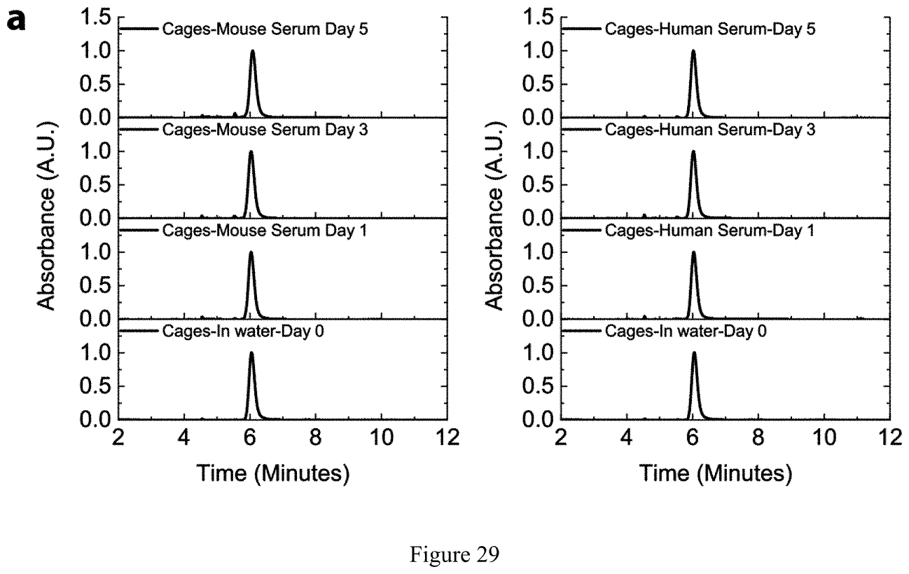

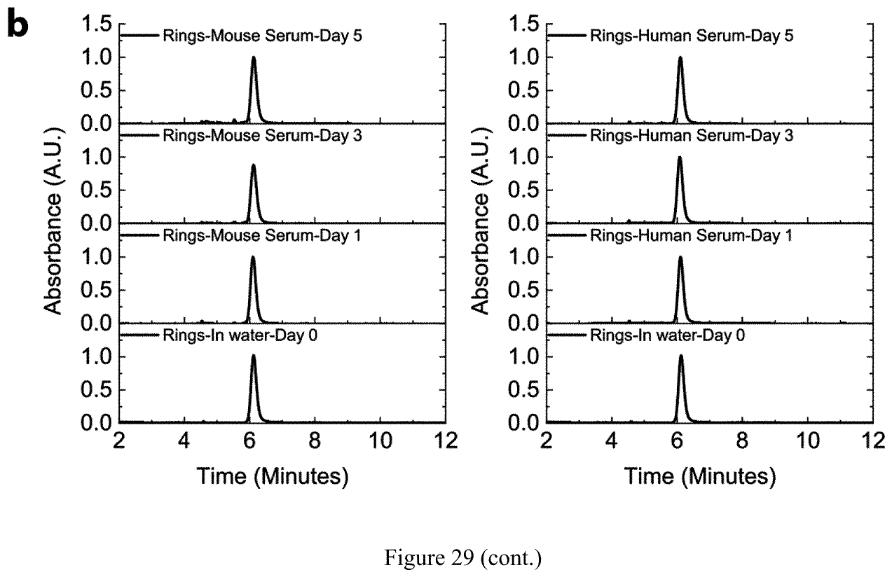

While no systematic dependence of liver (or spleen) uptake at 1 week p.i. on physical particle size was found, uptake strongly correlated with FCS measured diffusion coefficients and (equivalent) hydrodynamic sizes derived therefrom (FIG. 20c,d; FIG. 28). Diffusivity of spherical particles decreases with diameter, which is correlated with higher RES uptake (compare small and large dots with hollow spheres). Holes in nanoobjects change standard size-diffusivity relations. Silica cages have very similar shape, but larger (TEM) sizes than hollow spheres. Multiple holes in their surface lead to faster diffusion, however, which correlates with substantially lower liver (and spleen) uptake. Rings, while amongst the largest (TEM) diameter objects tested, because of their large hole and flat shape, have comparatively high diffusivity, correlating to low RES uptake. Extensive stability tests (Methods) in salt and protein solutions showed that particle aggregation or protein adsorption is minimal and cannot account for our observations (Tables 2 & 3, FIG. 29). The uptake-diffusivity correlation is not consistent with earlier models predicting higher particle sequestration probability in the liver with increasing diffusivity. Such simple models, in which diffusion competes with flow to transport particles to the liver sinusoid walls, while physically intuitive do not explicitly relate higher diffusivity to reduced particle residence time on wall surfaces, likely lowering cellular uptake by Kupffer and other cells.

TABLE 2
Stability of particles with different topologies in salt solution
over 7 days as measured by changes in hydrodynamic size via
FCS. Entries in column “Original Size” are from FCS
measurements right after synthesis, while entries in columns
“Size on Day 0” and “Size on Day 7” refer to FCS measurements
on the identical materials after storage in a refrigerator at 4° C.
for about a year. Within the error bars, particles sizes for different
topologies are essentially unchanged, both between original and
one year old particles, as well as on days 0 and 7 of the salt
solution treatment, confirming the high stability of the materials.

| | | Original | Size on | Size on |
|---|---|---|---|---|
| Excitation | | Size | day 0 | day 7 |
| Wavelength | Particle Type | (nm) | (nm) | (nm) |
| 445 nm | DEAC-Ring | 8.3 ± 0.2 | 7.6 ± 0.1 | 7.6 ± 0.1 |
| | DEAC-Cage | 11.3 ± 0.4 | 11.5 ± 0.5 | 10.9 ± 0.2 |
| | DEAC-Hollow | 14.2 ± 0.5 | 16.3 ± 1.3 | 14.9 ± 2.5 |
| 647 nm | Cy5-C′ dot | 5.2 ± 0.1 | 5.2 ± 0.1 | 5.3 ± 0.2 |

TABLE 3
Protein adsorption tests in mouse serum over 7 days for particles with
different topologies as measured by FCS particle size. Similar to Table 1, entries in column
“Original Size” are from FCS measurements right after synthesis, while entries in subsequent
columns “Day 0” to “Day 7” refer to FCS measurements on the identical materials after
storage in a refrigerator at 4° C. for about a year. The elevated diameters exclusively for rings
and cages may reflect smaller serum proteins hovering on the inside of these particles thereby
lowering their diffusivity rather than their physical adsorption, consistent with subsequent
HPLC-based stability tests on these materials to verify this hypothesis (see Fig. 29).

| Excitation | Particle | Original | Day 0 | Day 1 | Day 3 | Day 7 |
|---|---|---|---|---|---|---|
| Wavelength | Type | Size (nm) | (nm) | (nm) | (nm) | (nm) |
| 445 nm | DEAC-Ring | 8.3 ± 0.2 | 7.6 ± 0.1 | 8.8 ± 1.5 | 9.2 ± 1.2 | 7.6 ± 0.1 |
| | DEAC-Cage | 11.3 ± 0.4 | 11.5 ± 0.5 | 13.1 ± 0.6 | 12.3 ± 0.3 | 10.9 ± 0.2 |
| | DEAC-Hollow | 14.2 ± 0.5 | 16.3 ± 1.3 | 14.4 ± 1.2 | 14.7 ± 0.5 | 14.9 ± 2.5 |
| 647 nm | Cy5-C’ dot | 5.2 ± 0.1 | 5.2 ± 0.1 | 5.0 ± 0.1 | 5.2 ± 0.1 | 5.3 ± 0.2 |

The largest rings tested in mice had a diameter (TEM) of 13.5 nm (FIG. 25). A ˜1 nm thick PEG layer brings their size above 15 nm. They still showed favorable biodistribution with liver uptake at 1-week p.i. of only 2.6% ID/g. The 5.2 nm (FCS) dots with roughly 3-4 nm silica core diameter with similarly low liver uptake (i.e., 1.8% ID/g, FIG. 18a) have an estimated outer surface area of ˜40 nm². The large rings with a roughly 2 nm thick silica torus have a theoretical outer silica surface area of ˜230 nm², which increases to ˜440 nm² for a 12 nm cage, suggesting substantially improved loading capacities. Relative to ultrasmall spherical NPs, the combination of renal clearance, higher loading capacity, lower RES uptake, higher blood circulation times, and the ability to effectively “hide”, e.g., hydrophobic molecules on their inside, makes cage and ring topologies promising subjects for advanced applications in nanomedicine. Most notably, they allow inorganic nanomaterial designs to escape the ultrasmall NP size regime, i.e., the stringent limitations imposed by size requirements below 6 nm, in order to observe effective renal clearance and yield favorable biodistribution profiles.

METHODS. Chemicals and Materials: All materials were used as received. The succinimidyl ester of 7-diethylaminocoumarin-3-carboxylic acid (DEAC) was purchased from Anaspec. Cyanine5.0 maleimide (Cy5) was purchased from GE Healthcare. Hexadecyltrimethyl ammonium bromide (CTAB, ≥99%), tetramethyl orthosilicate (TMOS, ≥99%), 2.0 M ammonium hydroxide in ethanol, (3-aminopropyl)trimethoxysilane (APTMS, 97%), Hank's Balanced Salt Solution (HBBS) and anhydrous dimethyl sulfoxide (DMSO, ≥99%) were purchased from Sigma, Aldrich. (3-Aminopropyl) trimethoxysilane (APTES), 2-[methoxy(polyethyleneoxy)6-9 propyl] trimethoxysilane (PEG-Silane, 6-9 ethylene glycol units, PEG-silane (6EO)), (3-mercaptopropyl) trimethoxysilane (MPTMS, 95%), and methoxy triethyleneoxy propyl trimethoxysilane (PEG-silane, 3 ethylene glycol units, PEG-silane (3EO)) were obtained from Gelest. 1,3,5-trimethylbenzene (mesitylene/TMB, 99% extra pure) was purchased from Acros Organics. Deferoxamine-Bn-NCS-p (DFO-NCS, 94%) was purchased from Macrocyclics. Absolute anhydrous ethanol (200 proof) was purchased from Koptec. Glacial acetic acid was purchased from Macron Fine Chemicals. 5.0 M sodium chloride irrigation USP solution was purchased from Santa Cruz Biotechnology. Syringe filters (0.22 μm, PVDF membrane) were purchased from MilliporeSigma. Vivaspin sample concentrators (MWCO 30K) and Superdex 200 prep grade were obtained from GE Health Care. Snakeskin dialysis membranes (MWCO 10K) were purchased from Life Technologies. Deionized (DI) water was generated using Millipore Milli-Q system (18.2 MΩ·cm). Glass bottom microwell dishes for FCS were obtained from MatTek Corporation. Carbon film coated copper grids for TEM were purchased from Electron Microscopy Sciences. Xbridge Peptide BEH C18 Column (300 Å, 5 μm, 4.6 mm×50 mm, 10K-500K) was purchased from Waters Technologies Corporation. Human serum and mouse serum were purchased from BioIVT. UHPLC grade acetonitrile was purchased from BDH.

Synthesis of Silica Nanoparticles with Spherical Shape: Fluorescent core-shell silica nanoparticles with spherical shape were synthesized in aqueous solution as described previously. Briefly, Cy5 maleimide was conjugated to MPTMS via thiol-maleimide click-chemistry (1:23 ratio) a day prior to synthesis in a glove box. On the first day of particle synthesis for a 10 mL reaction batch, 68 μL TMOS and 0.367 μmol Cy5 dye-conjugate were added dropwise into 0.002 M ammonium hydroxide solution under stirring at 600 r.p.m. at room temperature resulting in the smallest (˜5 nm diameter) nanoparticles. For larger particle sizes, synthesis temperature was increased up to 80° C. as described previously. The following day, 100 μL PEG-silane (6EO) was added into the reaction solution, which was left stirring overnight at room temperature. The next day, in order to achieve full covalent attachment of PEG-silane molecules onto the silica core surface, the reaction solution was heated at 80° C. overnight without stirring. The solution was then cooled down to room temperature, and 2 μL APTMS was added at 600 r.p.m., while stirring at room temperature enabling post-PEGylation surface modification by insertion (PPSMI). The following day, 0.42 mmol of DFO-NCS chelator was added to the solution to react with primary amines on the silica surface via amine-NCS conjugation.

Synthesis of Inorganic Nanoparticles with Ring, Cage and Hollow Bead Topologies: Fluorescent silica cages and rings were synthesized in aqueous solution via micelle templating as described previously, whereas the synthesis of hollow beads, described herein, has not been reported. Briefly, succinimidyl ester derivative of DEAC dye was conjugated with APTES via amine-ester conjugation-chemistry (1:25 ratio) a day prior to synthesis in a glove box. On the first day of particle synthesis for a 10 mL reaction batch, CTAB (125 mg for cages, 50 mg for hollow beads, and 83 mg for rings) was dissolved into 10 mL of 0.002 M ammonium hydroxide solution under stirring at 600 r.p.m. at 30° C. for 1 hour before the addition of 100 μL TMB to swell the micelles, which was followed by stirring for another hour. TMOS (100 μL for cages, 800 μL for hollow beads, and 68 μL for rings) and 0.2 μmol DEAC-dye conjugate were then added dropwise to the reactions, except for hollow beads, which required a post-PEGylation fluorescent dye functionalization on the particle surface due to the high concentration of silica precursor used in the bead synthesis causing aggregation and making it hard to successfully functionalize the hollow beads with fluorescent dyes using ester chemistry. The following day, 6EO PEG-silane (150 μL for cages, 1200 μL for hollow beads, and 100 μL for rings) was added into the reaction solutions, which were left stirring overnight at 30° C. The next day, in order to achieve full covalent attachment of PEG-silane molecules onto the silica surface, the solutions were heated at 80° C. overnight without stirring. The reaction solutions were then cooled down to room temperature. The hollow bead particle sample, specifically at this step, was centrifuged at 4300 r.p.m. three times to remove larger aggregates. Subsequently, samples were syringe-filtered (MWCO 0.2 μm, PTFE), and transferred into a dialysis membrane (MWCO 10K). The samples were dialyzed in 200 mL of ethanol/deionized water/glacial acetic acid solution (500:500:7 volume ratio), and the acid solution was changed once a day for three days to remove CTAB micelles from the inner pores of the silica NPs, as well as to remove unreacted reagents. Following acid dialysis, the samples were transferred into 5 L deionized water, and the deionized water was refreshed once a day for three days to remove ethanol and acetic acid solvents.

Following these dialysis treatments, the reaction batches were transferred back into a round-bottom flask, and 100 μL of PEG-silane (3EO) was added into the reactions under stirring overnight in order to further PEGylate the inside silica surfaces, which had been covered by micelles during the first PEGylation step. This secondary PEGylation was also followed by heating at 80° C. overnight. The day following the heating step, 2 μL APTMS was added into the reactions at 600 r.p.m. at room temperature for PPSMI. For the hollow beads following the PPSMI step, 0.697 μmol free DEAC dye with ester chemistry was added into the solution on the next day in order to click dye to the surface amines, while this additional step was skipped for cages and rings since they were already functionalized with DEAC dye on day one of the particle synthesis. Following the PPSMI step, 0.42 mmol of DFO-NCS chelator was added to the solutions to react with primary amines on the nanoparticle surface via amine-NCS conjugation. After the functionalization with DFO, samples were heated at 80° C. overnight and subsequently purified as described below.

Sample Purification: After syntheses of all inorganic NPs, reaction batches were transferred into dialysis membranes (MWCO 10K) for dialysis in deionized water overnight prior to syringe-filtration (MWCO 0.2 μm, PTFE), after which they were concentrated using spin filters (Vivaspin 20 MWCO 30K) via centrifugation (Eppendorf 5810R) at 4300 r.p.m. for 45 min. Gel permeation chromatography (GPC) was performed on the concentrated samples on a GPC column packed with Superdex 200 prep grade resin using 0.9 wt. % sodium chloride saline as buffer solution, as described previously. NPs were separated from the aggregation products and un-reacted reagents via GPC fractionation, and collected samples were run by GPC again to check for sample purity via the occurrence of a single-peak chromatogram. This resulted in the GPC control runs reported in the data sets comparing different topologies (FIG. 21).

Characterization of Inorganic Nanoparticles: Fluorescence correlation spectroscopy (FCS) measurements were performed to determine size and concentration of different NPs using a home-built setup as described previously. Diffusion coefficients, D, were obtained from measured correlation times, τ_{D}, using the geometrical factor, ω_{xy}, representing the radius of the FCS focal spot, according to equation (1):

$$D = \frac{{ω}_{xy}^{2}}{4 {τ}_{D}}$$

In turn, D was used to determine the (equivalent) hydrodynamic diameter, d, of the particles, i.e. the diameter of a(n) (equivalent) spherical particle derived from the Stokes-Einstein relation, equation (2):

$$d = 2 \frac{{k}_{B} T}{6 π η D}$$

where k_{B} is Boltzmann constant, T is temperature, and η is the solution viscosity.

A Varian Cary 5000 spectrophotometer was used to measure UV-vis absorption spectra of the samples in order to calculate, together with concentration information from FCS data analysis, the number of dyes and DFO chelators per particle by deconvolution as described previously. Transmission and cryo-electron microscopy (TEM/cryo-EM) were performed on particle samples using a FEI Tecnai T12 Spirit microscope operated at 120 kV. Cryo-EM was performed on cage and ring samples as described previously.

To study the integrity of cages and rings after circulation and excretion from mice injected with 250 μL of 15 μM NPs, urine specimens were collected at 2-hour post i. v. injection time point from the mouse bladder while the animal was under anesthesia. After extraction, the urine sample was immediately diluted with deionized water for TEM sample preparation. For samples prepared from urinary specimens, typically more than 15 TEM images were taken per nanoparticle. These images were then averaged to increase the signal-to-noise ratio, as shown in FIG. 26 and described elsewhere.

The zeta-potential of particles with different topologies was measured with a Malvern Zetasizer Nano-ZS operated at neutral pH in deionized water at 20° C. after up-concentrating particle solutions via spin-filters to obtain the desired signal-to-noise ratios as described elsewhere. Each sample was measured three times and results were averaged.

Stability Tests of Inorganic Nanoparticles via FCS: For salt solution stability experiments, 10 μL of a 15 μM nanoparticle suspension was mixed with 1 mL of Hanks' Balanced Salt Solution (HBSS) in a 10 mL centrifuge tube. The tube was placed in a humidity-controlled cell incubator set to 37° C. with 5% CO₂. After 7 days of incubation, 1 μL of nanoparticle-salt solution was diluted into 180 μL of DI water on a 35 mm MatTek No. 1.5 coverslip dish with a 10 mm well (P35G-1.5-10-C). The dish was placed on a 63× water immersion microscope objective and solutions characterized using FCS.

For protein adsorption experiments, a 10 vol. % mouse serum solution was used. To that end, 20 μL of nanoparticle sample at 15 μM concentration was first transferred to a 2 mL screw top centrifuge tube and then diluted with 250 μL of DI water. After adding 30 μL of mouse serum, the centrifuge tube was kept rotating at 37° C. in a cell incubator. For each protein adsorption test, a 40 μL aliquot of the nanoparticle-serum mixture was transferred to a 1.5 mL centrifuge tube followed by the addition of 40 μL chilled acetonitrile (−30° C.) to precipitate the serum proteins. The resulting cloudy mixture was then centrifuged for 20 minutes at 10,000 RCF and 20 μL of the separated supernatant was transferred into a new 1.5 mL centrifuge tube. Using a 35 mm MatTek No. 1.5 coverslip dish with a 10 mm well (P35G-1.5-10-C), 1 μL of nanoparticle-acetonitrile solution was diluted into 180 μL of DI water. The dish was then placed on a 63× water immersion microscope objective and solutions characterized using FCS.

Stability Tests of Inorganic Nanoparticles via HPLC: HPLC Method: All injections were performed with a standardized 60 μL injection volume. The columns used were 50 mm Waters Xbridge Peptide separation columns with 300 Å pore size and 5 μm particle size. Samples were injected onto the column that had been equilibrated with a solvent composition of 95% deionized water with 0.01 volume percent trifluoroacetic acid (TFA) and 5% acetonitrile. After sample injection, a gradient elution profile from the 95:5 composition to a composition of 15% deionized water with 0.01% TFA and 85% acetonitrile was carried out over 8 minutes. The composition was then changed to 95% acetonitrile over 2 minutes. This process was followed by a cleaning and equilibration step before injection of a new sample.

Stability Test: 7.5 μM solutions of inorganic NPs were incubated with 10% by volume serum prepared as follows: First, 150 μL of 15 μM particle solution was aliquoted into a 1.5 mL microcentrifuge tube and diluted with 120 μL of deionized water to bring the total volume of the solution to 270 μL. Finally, 30 μL of either mouse or human serum was added. The tube was closed, para-filmed, and shaken at 300 rpm at 37° C., with 40 μL aliquots taken out at each time point of interest for analysis. For HPLC analysis, 40 μL of cold acetonitrile was added to each aliquot to precipitate serums proteins. Then the aliquots were centrifuged at 10000 rpm for 30 minutes to pellet the precipitated proteins. A 40 μL aliquot of the supernatant was taken and deposited into a Waters Total Recovery HPLC vial. In order to dilute the acetonitrile in the sample vial, an additional 40 μL of deionized water was added to each vial and mixed prior to HPLC injection.

⁸⁹Zr Radiolabeling of DFO-functionalized Inorganic Nanoparticles: For chelator-based ⁸⁹Zr labeling, 1.5 nmol of DFO-functionalized samples were mixed with 1 mCi of ⁸⁹Zr-oxalate in HEPES buffer (pH 8) at 37° C. for 60 min; final labeling pH was kept around 7-7.5. The labeling yield was monitored by radio ITLC. An EDTA challenge process was then introduced to remove any non-specifically bound ⁸⁹Zr to the silica NP surface. As synthesized ⁸⁹Zr-DFO-NP samples were then purified by using a PD-10 column with the final radiochemical purity quantified as 100% using ITLC.

Quantitative Renal and Hepatic Clearance Studies of Inorganic Nanoparticles: To study the renal and hepatic clearance of ⁸⁹Zr-DFO-functionalized silica nanoparticles with varying topologies, each healthy mouse (6-8 week-old female nude mouse) was injected with about 50 μCi (1.85 MBq) of ⁸⁹Zr-DFO-NP, and housed individually in metabolic cages. At varied post i. v. injection time points (i.e., at 4, 24, 48, 72, 120 and 168 h), the cumulative radioactivity in mouse urine and feces were measured separately using a CRC®-55tR Dose Calibrator and presented as % ID (mean±SD).

All animal experiments were performed in accordance with protocols approved by the Institutional Animal Care and Use Committee of the Memorial Sloan Kettering Cancer Center (MSKCC) and followed National Institutes of Health (NIH) guidelines for animal welfare.

In-Vivo PET Imaging and Ex-Vivo Biodistribution Studies for Inorganic Nanoparticles: For PET imaging, mice were i.v. injected with ˜300 μCi (11.1 MBq) ⁸⁹Zr-DFO-NP. PET imaging was performed in a small-animal PET scanner (Focus 120 microPET; Concorde Microsystems) at 1, 24, 48, 72 h and 168 h (one week) post i.v. injection. Image reconstruction and region-of-interest (ROI) analysis of the PET data were performed using IRW software, with results presented as the percentage of the injected dose per gram of tissue (% ID/g). On day 7, post i.v. injection, accumulated activity in major organs was assayed by an Automatic Wizard² γ-Counter (PerkinElmer), and presented as % ID/g (mean±SD). Biostatistics: Biodistribution and clearance profiles were compared across sizes, topologies, and organs using a linear model with interactions. Significance was evaluated using a Wald test and maximum likelihood estimates.

Mechanical model for particle deformation: The glomerular capillary pressure, P_{gc}, has been measured in rodents (rats) and is 88 mm Hg=11,732 Pa, i.e., around 10 kPa. Arguments supporting the hypothesis that this is enough to deform the nanoparticles, in particular those with ring and cage topologies, follow the subsequent analysis: the silica structure of rings and cages (and, we suspect, even of hollow spheres), overall, is not homogeneous, but rather consists of silica clusters of around 2 nm in diameter, that are subsequently connected via additional Si—O—Si bond formation (vide supra). Careful TEM studies, e.g., of the rings, suggest that this results in what could be described as a “pearl-chain” type structure, as opposed to a homogeneous torus shape (see also TEM images in FIG. 17).

Within the thin links or bridges between individual silica clusters, the condensation degree of silica is expected to be even lower than that of regular C dots, most likely characterized predominantly by Q2 groups rather than Q3 groups (i.e. each silicon atom only has two rather than three bridging oxygens to other Si atoms, reflecting linear chain behavior). This suggests that the thin bridges have more the character of a cross-linked polysiloxane rather than that of highly cross-linked silica characterized predominantly by Q4 groups, i.e. they are compliant links. A typical representative of a polysiloxane is poly(dimethyl-siloxane) (PDMS). Crosslinked PDMS rubber has Young's modulus somewhere between 360-870 kPa; significantly more compliant than Q4-dominated silica, for which E≈72 GPa.

The modulus of PDMS would still be one to two orders of magnitude too high, however, to explain particle deformation during renal excretion, if the rings were considered to have a uniform cross section of 2 nm. In contrast, in a pearl-chain, bending deformation is concentrated in the thin links rather than the pearls. As demonstrated by a model calculation (FIG. 27), the bending moment, M, is exquisitely sensitive to the diameter of these links (M ∝r⁴). Reducing the diameter of the links to about 50%, 30%, or 20% of the regular diameter of the ring torus decreases the bending modulus by 1, 2, or 3 orders of magnitude, respectively. Such diameters would still allow multiple linear chains to connect two neighboring clusters, enough to provide stability and elastic compliance. In summary, in the “pearl-chain” picture, the bending modulus of the rings is substantially reduced by having thin and compliant links. Since the formation mechanism of rings and cages (as well as hollow spheres) is similar, we expect that such thin and compliant links between silica clusters facilitate their deformation during the glomerular filtration process responsible for the observed renal clearance of these particles.

Although the present disclosure has been described with respect to one or more particular example(s), it will be understood that other examples of the present disclosure may be made without departing from the scope of the present disclosure.

1. A method of making inorganic nanocages, comprising
forming a reaction mixture comprising
one or more precursor(s);
one or more surfactant(s);
one or more pore expander(s); and
holding the reaction mixture at a time (t¹) and temperature (T¹), whereby inorganic nanocages having an average size of a longest dimension less than 30 nm are formed; and
optionally, adding a terminating agent to the reaction mixture.

2. The method of claim 1, wherein
the one or more surfactant(s) is/are chosen from C₁₀ to C₁₈ alkyltrimethylammonium halides, sodium dodecyl sulfate (SDS), N-myristoyl-L-glutamic acid (C14GluA), and combinations thereof, and/or
the one or more pore expander(s) is/are chosen from trialkylated benzene, polymer monomers, hydrophobic solvents, and combinations thereof.

3. The method of claim 1, wherein the one or more surfactant(s) is/are present in the reaction mixture at a concentration ranging from 1 mg/mL to 50 mg/mL and the one or more pore expander(s) is/are present at a concentration ranging from 3 mg/mL to 100 mg/mL.

4. The method of claim 1, wherein the molar ratio of the one or more surfactant(s) to the one or more pore expander(s) is 1:100 to 10:1.

5. The method of claim 1, wherein the one or more precursor(s) is/are one or more non-metal oxide precursor chosen from silica precursors, alkyltrialkoxysilanes precursors, functionalized non-metal oxide precursors, and combinations thereof.

6. The method of claim 5, wherein at least one of non-metal oxide precursors comprises one or more functional group(s).

7. The method of claim 5, wherein the terminating agent is a PEG-silane.

8. The method of claim 7, wherein before or after the PEG-silane conjugate is added,
adding a PEG-silane conjugate comprising a ligand is added at room temperature to the reaction mixture,
holding the resulting reaction mixture at a time (t²) and temperature (T²), and
subsequently heating the resulting reaction mixture at a time (t³) and temperature (T³), whereby inorganic nanocages surface functionalized with PEG groups comprising a ligand are formed.

9. The method of claim 7, wherein at least a portion of or all of the PEG-silane has a reactive group on a terminus of the PEG group opposite the terminus conjugated to the silane group of the PEG-silane conjugate and after formation of the inorganic nanocages surface functionalized with PEG groups having a reactive group, and, optionally, PEG groups, are reacted with a second ligand functionalized with a second reactive group thereby forming inorganic nanocages surface functionalized with polyethylene groups functionalized with a second ligand and, optionally, PEG groups.

10. The method of claim 7, wherein at least a portion of or all of the PEG-silane has a reactive group on a terminus of the PEG moiety opposite the terminus conjugated to the silane moiety of the PEG-silane conjugate and after formation of the inorganic nanocages surface functionalized with PEG groups and, optionally having a reactive group, and, optionally, PEG groups, are reacted with a second ligand functionalized with a second reactive group thereby forming inorganic nanocages surface functionalized with polyethylene groups functionalized with a second ligand and, optionally, PEG groups.

11. The method of claim 5, wherein the reaction mixture further comprises a solvent and the solvent is water and the pH of the reaction mixture is 6 or greater.

12. The method of claim 1, wherein the one or more precursor(s) is/are one or more transition metal precursor(s) chosen from transition metal salts, transition metal alkoxides, transition metal coordination complexes, organometallic compounds, and combinations thereof.

13. The method of claim 12, wherein the transition metal salts are gold salts, silver salts, palladium salts, platinum salts, zirconium salts, iron salts, rhodium salts, copper salts, nickel salts, tantalum salts, hafnium salts, niobium salts, and combinations thereof.

14. The method of claim 12, wherein the terminating agent is a reducing terminating agent.

15. The method of claim 14, wherein the reducing terminating agent is chosen from tetrakis(hydroxymethyl)phosphonium chloride (THPC), bis[tetrakis(hydroxymethyl)phosphonium] sulfate (THPS), and combinations thereof.

16. The method of claim 1, wherein the one or more precursor(s) is/are one or more transition metal oxide precursor(s) chosen from transition metal alkoxides, transition metal salts, and combinations thereof.

17. The method of claim 16, wherein the transition metal alkoxides are vanadium alkoxides, titanium alkoxides, niobium alkoxides, zirconium alkoxides, tantalum alkoxides, hafnium alkoxides, copper alkoxides, nickel alkoxides, iron alkoxides, and combinations thereof.

18. The method of claim 1, wherein at least a portion of a surface is functionalized.

19. The method of claim 1, wherein the method further comprises isolation/separation of at least a portion of the inorganic nanocages from the reaction mixture.

20. An inorganic nanocage having a longest dimension less than 30 nm comprising an inorganic material, the inorganic nanocage comprising:
an interior and an exterior, wherein the interior and the exterior each have a surface;
vertices having a longest dimension of about 1 nm to about 5 nm;
arms connecting adjacent/nearby vertices having a longest dimension of about less than 1 nm to about 3 nm; and
apertures, which can connect the exterior space to the interior space having a longest dimension of about 1 nm to about 10 nm.

21. The inorganic nanocage of claim 20, wherein the interior surface and/or exterior of the inorganic nanocage is functionalized/modified with at least one functional group, wherein when there is more than one functional group, the functional groups are the same or different, or some are the same and some are different.

22. The inorganic nanocage of claim 21, wherein the at least one functional group is chosen from peptide groups, nucleic acid groups, drug groups, sensor ligands, antibody groups, antibody fragment groups, groups comprising a radioisotope, and combinations thereof.

23. The inorganic nanocage of claim 20, wherein the inorganic material is chosen from non-metal oxides, transition metal oxides, metals, and combinations thereof.

24. The inorganic nanocage of claim 23, wherein the non-metal oxide is chosen from silicon oxide and aluminosilicate.

25. The inorganic nanocage of claim 23, wherein the transition metal oxide is chosen from vanadium oxide, titanium oxide, niobium oxide, iron oxide, copper oxide, nickel oxide, hafnium oxide, zirconium oxide, tantalum oxide, and combinations thereof.

26. The inorganic nanocage of claim 23, wherein the transition metal is chosen from silver, gold, palladium, platinum, rhodium, and combinations thereof.

27. The inorganic nanocage of claim 20, wherein the inorganic nanocage is dodecahedral (5¹²), icosahedral, cubic, hexahedral, tetrahedral, octahedral, tetrakaidecahedral, pentakaidecahedral, hexakaidecahedron, rhombic dodecahedral, trapezo-rhombic, buckyball-like (5¹²6²⁰), 3³4³, 4⁴5⁴, 4³5⁶6³, 3³4³5⁹, 5¹²6², 4⁶6⁸5¹²6³, 5¹²6⁴, 4³5⁹6²7³, or 5¹²6⁸.

28. The inorganic nanocage of claim 20, wherein the inorganic nanocage has a specific surface area 500 to 800 square meter per gram.

29. The inorganic nanocage of claim 20, wherein the inorganic nanocage is used as a catalyst, drug delivery agent, diagnostic agent, as a therapeutic agent, a theranostic agent, or a combination thereof.

30. The inorganic nanocage of claim 20, wherein the inorganic nanocage has a longest dimension of 5 to 15 nm.

31. A composition comprising one or more inorganic nanocage(s) of claim 20.

32. A method for imaging of a region within an individual comprising:
administering to the individual the composition of claim 31, wherein the inorganic nanocages comprise one or more dye molecule(s), one or more radioisotope(s), one or more iodide(s), or a combination thereof;
directing excitation electromagnetic radiation into the individual, thereby exciting at least one of the one or more dye molecule(s);
detecting excited electromagnetic radiation, the detected electromagnetic radiation having been emitted by said dye molecules in the individuals as a result of excitation by the excitation electromagnetic radiation; and
processing signals corresponding to the detected electromagnetic radiation to provide one or more image(s) of the region within the individual.

33. The method of claim 31, wherein the imaging is optical, PET imaging, CT imaging, or a combination thereof.

34. A method of treating cancer in an individual comprising administering to the individual a therapeutically effective amount of a composition of claim 31, wherein the individual's cancer is treated.

35. The method of claim 34, wherein at least a portion of the inorganic nanocage(s) comprises a drug and at least a portion of the drug is released from the inorganic nanocage(s).

36. The method of claim 34, wherein at least a portion of the inorganic nanocage(s) comprises one or more display group(s) that target(s) the cancer.

37. The method of claim 34, further comprising visualization of at least a portion of the cancer using optical imaging, PET imaging, CT imaging, or a combination thereof.

38. The method of claim 34, further comprising treatment of the individual with one or more known cancer therapy/therapies in conjunction with administration of the inorganic nanocage(s).

39. The method of claim 34, wherein the cancer is a solid tumor.

40. The method of claim 39, wherein the cancer is chosen from brain cancers, melanomas, prostate cancer, breast cancer, lung cancer, and combinations thereof.

41. The method of claim 34, wherein the individual is a human individual or a non-human individual.
